(19) United States
(12) Patent Application Publication
(10) Pub. No.: US 2020/0009262 A1
(43) Pub. Date: Jan. 9, 2020

(54) TARGETED CONSTRUCTS AND FORMULATIONS THEREOF

(71) Applicant: TARVEDA THERAPEUTICS, INC., Watertown, MA (US)

(72) Inventors: Brian H. White, Malden, MA (US); Benoît Moreau, Newton, MA (US); Mark T. Bilodeau, Waltham, MA (US); Sudhakar Kadiyala, Newton, MA (US)

(21) Appl. No.: 16/301,144

(22) PCT Filed: May 12, 2017
(86) PCT No.: PCT/US2017/032366

§ 371 Date: Nov. 13, 2018

Related U.S. Application Data
(60) Provisional application No. 62/336,120, filed on May 13, 2016.
(60) Provisional application No. 62/476,123, filed on Mar. 24, 2017.

(51) Int. Cl. A61K 47/66; A61K 47/64; A61K 47/55; A61K 47/42

(52) U.S. Cl. CPC A61K 47/66; A61K 47/42; A61K 47/55; A61K 47/64

(57) ABSTRACT

Targeted constructs and pharmaceutical formulations thereof, comprising at least one conjugate of an active agent such as a therapeutic, prophylactic, or diagnostic agent attached to a targeting moiety via an optional internal linker moiety have been designed which can provide improved temporospatial delivery of the active agent and/or improved biodistribution. Methods of making the targeted constructs and the formulations thereof are provided. Methods of administering the formulations to a subject in need thereof are provided, for example, to treat or prevent cancer or infectious diseases.

### REFERENCED TO RELATED APPLICATIONS

The present application claims priority to U.S. Provisional Patent Application No. 62/336,120, filed May 13, 2016, and U.S. Provisional Patent Application No. 62/476,123, filed Mar. 24, 2017, the contents of each of which are herein incorporated by reference in their entirety.

### FIELD OF THE INVENTION

This invention is generally in the field of conjugates and formulations for drug delivery.

### BACKGROUND OF THE INVENTION

Developments in therapeutic small molecule drugs and therapeutic peptide drugs are generally directed towards improving the pharmaceutical properties of the drugs and, in some cases, improving their poor pharmacokinetics and other factors. For example, therapeutic peptides (<50 amino acid) have short in vivo half-life (t_{1/2} of 2-30 min) and have been conjugated to macromolecule drug carriers, such as polyethylene glycol (PEG), antibody Fc domain and human serum albumin (HSA or albumin) to extend their half-life, so that the therapeutic potential of the peptides may be realized without frequent administration and/or high doses. Macromolecule drug carriers also have an enhanced permeability and retention (EPR) effect and accumulate preferentially in tumor tissues and inflame tissues. However, the macromolecule drug carriers may introduce steric hindrance and comprise in vivo efficacy. Further, macromolecule drug carriers may be detrimental to the penetration of the drugs into a target tissue.

Accordingly, there is a need in the art for improved drug targeting, pharmacokinetic and delivery without interfering with efficacy of the drug.

### SUMMARY OF THE INVENTION

Applicants have created targeted constructs that comprise at least one conjugate of a targeting moiety and an active agent connected by an optional internal linker moiety. In one embodiment, the targeted constructs may comprise an external linker connected to a reacting group, wherein the reacting group reacts with a functional group on a protein or an engineered protein or derivatives/analogs/mimics thereof. Alternatively, the targeted constructs may comprise an external linker connected to a pharmacokinetic modulating unit. The targeted constructs may also be an assembly of at least two conjugates. The targeted constructs that contain a reacting group for attachment to a protein or an engineered protein, contain a pharmacokinetic modulating unit, or are an assembly of at least two conjugates are useful for improving targeted delivery and pharmacokinetics of active agents.

Methods of making and using the targeted constructs are provided. Methods are also provided for treating a disease or condition, the method comprising administering a therapeutically effective amount of targeted constructs of the present invention to a subject in need thereof. In some embodiments, the conjugates in the targeted constructs are targeted to a cancer or hyperproliferative disease, for example, lymphoma (e.g., non-Hodgkin's lymphoma), renal cell carcinoma, prostate cancer, ovarian cancer, breast cancer, colorectal cancer, neuroendodrine cancer, endometrial cancer, pancreatic cancer leukemia, lung cancer, glioblastoma multiforme, stomach cancer, liver cancer, sarcoma, bladder cancer, testicular cancer, esophageal cancer, head and neck cancer, and leptomeningeal carcinomatosis.

### BRIEF DESCRIPTION OF THE DRAWINGS

FIG. 1 is a non-limiting example of the design the targeted constructs of the present invention.

FIG. 2 is a scheme of an albumin-binding conjugate targeting NTSR1.

FIG. 3 shows concentration changes of Compounds 3 and 4 in rat plasma were shown in FIG. 3.

FIG. 4 shows average tumor volume changes after treatment with vehicle, irinotecan, Compound 4, Compound 7, Compound 3, Compound 8, Compound 5 and Compound 6 in an SW-48 xenograft model.

FIG. 5 shows blood pressure levels of in Sprague Dawley rats after treatment with vehicle, Compound 3, Compound 4, or neurotensin.

### DETAILED DESCRIPTION OF THE INVENTION

Applicants have created targeted constructs to deliver an active agent to a diseased tissue such as tumor tissues and to improve pharmacological and/or pharmacokinetic properties of the active agent. The targeted constructs may comprise at least one conjugate of an active agent and a targeting moiety connected with an optional internal linker moiety. The targeted constructs may further comprise at least one external linker connected to a reacting group that reacts with a functional group on a protein or an engineered protein or derivatives/analogs/mimics thereof, or comprise at least one external linker connected to a pharmacokinetic modulating unit. The targeted constructs may also be an assembly of at least two conjugates connected to each other with external linkers. The amount of the active agent delivered at an action site is increased and the active agent's systemic toxicity is decreased. These targeted constructs combine passive targeting of macromolecules and active targeting of the targeting moiety. They also have extended half-life and increased penetration in diseased tissues. Controlled and sustained release of the active agent can be achieved with targeted constructs of the present invention.

An internal linker moiety or an internal linker, as used herein, refers to a linker in a conjugate, wherein the internal linker connects an active agent and a targeting moiety. The internal linker may be non-cleavable or cleavable. In particular, the internal linker can be cleaved in an intracellular fashion after uptake of the conjugate by the targeted receptor, including but not limited to, pH-sensitive linkers for release in organelles with lowered pH, or linkers cleaved by intracellular proteases such as Cathepsin B, or disulfide linkers.

An external linker, as used herein, refers to the linker that is not part of a conjugate, wherein the external linker connects the conjugate with another chemical group, such as a reacting group for attachment to a protein or an engineered protein, or a pharmacokinetic modulating unit. The external linker may be non-cleavable or cleavable. In particular, the external linker can be cleaved in an extracellular fashion, including but not limited to, pH-sensitive linkers for release in the lower pH of the tumor environment, or linkers cleaved by extracellular proteases such as matrix metalloproteinases, or hypoxia-activated linkers.

The internal linker and the external linker may be the same or different. In some embodiments, the internal linker and the external linker are both cleavable, but are different from each other. Preferably, the external linker is less stable than the internal linker in conditions expected to be operative in the tumor, i.e., tumor microenvironment. Conditions operative in the tumor may include low pH, a reducing environment, or in the presence of certain enzymes such as matrix metalloproteinases. Alternatively, the external linker is cleavable in excellular manner and the internal linker is cleavable in intracellular manner. As a result, the external linker is cleaved in an extracellular manner in a tumor microenvironment by a pH-dependent or hypoxia-dependent cleavage that relies upon conditions in the tumor microenvironment or by extracellular proteases such as matrix metalloproteinases, while the internal linker cleaves inside cells. Therefore, a conjugate is released from a targeted construct assembly, or separated from a protein, an engineered protein, or a pharmacokinetic modulating unit before the release of an active agent. Not willing to be bound to any theory, the conjugates penetrate deeper and faster into the tumor than the targeted construct assembly, because the conjugates have a smaller molecular weight once released. The active agent in the conjugate is only released inside cells.

In some embodiments, the relative stabilities or cleavage rates of the internal linker and the external linker can be evaluated in a tumor microenvironment. The cleavage rate of the external linker may be at least 25%, at least 50%, at least 100%, at least 150%, at least 2 folds, at least 3 folds, at least 4 folds, at least 5 folds faster than the internal linker in a tumor microenvironment.

A “cleavable” linker, as used herein, refers to any linker which can be cleaved physically or chemically. Examples for physical cleavage may be cleavage by light, radioactive emission or heat, while examples for chemical cleavage include cleavage by re-dox-reactions, hydrolysis, pH-dependent cleavage or cleavage by enzymes.

Macromolecules, as used herein, refer to large molecules with molecule weight over 10 KDa, including but not limited to proteins, lipids, nucleic acids, polysaccharides, nanoparticles, polymers, and dendrimers.

The term “reacting group,” as used herein, refers to any chemical functional group capable of reacting with another functional group to form a covalent bond.

Pharmacokinetics, as used herein, describes what the body does to a drug. It may refer to the movement of drug into, through, and out of the body—the time course of its absorption, bioavailability, distribution, metabolism, and excretion. Drug pharmacokinetics determines the onset, duration, and intensity of a drug's effect.

In some embodiments, the targeted construct comprising at least one conjugate has a plasma clearance rate less than about 5%, 10%, 20%, 30%, 40%, or 50% of the clearance rate of the conjugate itself without an external linker (i.e., not connected to any reacting group or any pharmacokinetic modulating unit).

In some embodiments, the targeted construct comprising at least one conjugate has a plasma area under the curve (AUC) that is at least about 25%, 50%, 75%, 100%, 200%, or 500% more than the AUC of the conjugate itself without an external linker (i.e., not connected to any reacting group or any pharmacokinetic modulating unit).

As used herein, “controlled release” refers to a pharmaceutical composition or compound release profile that conforms to a particular pattern of release to effect a therapeutic outcome.

As used herein, “sustained release” refers to a pharmaceutical composition or compound that conforms to a release rate over a specific period of time. The period of time may include, but is not limited to, hours, days, weeks, months and years.

As used herein, “toxicity” refers to the capacity of a substance or composition to hit off targets and/or be harmful or poisonous to a cell, tissue, organ tissue, vasculature, or cellular environment. Low toxicity refers to a reduced capacity of a substance or composition to be harmful or poisonous to a cell, tissue, organ tissue or cellular environment. Such reduced or low toxicity may be relative to a standard measure, relative to a treatment or relative to the absence of a treatment.

Toxicity may further be measured relative to a subject's weight loss where weight loss over 15%, over 20% or over 30% of the body weight is indicative of toxicity. Other metrics of toxicity may also be measured such as patient presentation metrics including lethargy and general malaiase. Neutropenia or thrombocytopenia may also be metrics of toxicity.

Biomarkers of toxicity include elevated AST or ALT levels, neurotoxicity, kidney damage, GI damage and the like.

In addition, the toxicity of targeted constructs comprising at least one conjugate containing a targeting moiety linked to an active agent for cells that do not express the target of the targeting moiety is predicted to be decreased compared to the toxicity of the active agent alone. Without committing to any particular theory, applicants believe that this feature is because of the ability of the conjugated active agent to enter a cell is decreased compared the ability to enter a cell of the active agent alone. Accordingly, the conjugates comprising an active agent as described herein generally have reduced toxicity for cells that do not express the target of the targeting moiety and at least the same or increased toxicity for cells that express the target of the targeting moiety compared to the active agent alone.

Furthermore, the targeted constructs of the present invention may improve half life of the active agent and prevent the active agent from degradation and/or compromise before the active agent reaches the target site.

It is an object of the invention to provide improved compounds, compositions, and formulations for temporospatial drug delivery.

It is further an object of the invention to provide methods of making improved compounds, compositions, and formulations for temporospatial drug delivery.

It is also an object of the invention to provide methods of administering the improved compounds, compositions, and formulations to individuals in need thereof.

### I. Targeted Constructs Comprising at Least One Conjugate

Targeted constructs of the present invention comprise at least one conjugate, wherein the conjugate includes an active agent or prodrug thereof attached to a targeting moiety by an optional internal linker moiety. Targeted constructs of the present invention 1). are assemblies of at least two conjugates connected to each other with external linkers, 2). comprise a conjugate and at least one reacting group attached via an external linker, wherein the reacting group reacts with a functional group on a protein or an engineered protein or a polymer or derivatives/analogs/mimics thereof, or 3). comprise a conjugate and at least one pharmacokinetic modulating unit attached via an external linker. The half-life of the targeted constructs may be at least about 25%, 50%, 75%, 100%, 200%, or 500% more than the half-life of the conjugate without an external linker.

### Embodiment 1

In one embodiment, the targeted constructs are an assembly of at least two conjugates: (Conjugates)ₙ, n≥2. The conjugates may be attached to each other via covalent bonds or linkers. Alternatively, the conjugates may be attached to each other via ionic bonds or other non-covalent bonds. These conjugates have a molecular weight of at least about 0.5 KDa, at least about 2 KDa, at least about 3 KDa or at least about 5 KDa. Generally, these conjugates have a molecular weight between about 0.5 KDa and about 30 KDa. Preferably, these targeted constructs have a molecular weight between about 1 KDa and about 20 KDa. These targeted constructs comprising the conjugates have a molecular weight of at least about 10 KDa, at least about 20 KDa, at least about 30 KDa or at least about 50 KDa. Generally, these targeted constructs have a molecular weight between about 10 KDa and about 30 KDa. Preferably, these targeted constructs have a molecular weight between about 10 KDa and about 20 KDa.

The external linkers connecting the conjugates may be cleavable linkers that allow release of the conjugates. The external linkers connecting the conjugates may be less stable than the internal linker moiety in the conjugates connecting the active agent and the targeting moiety. Hence, the conjugates are separated from each other, i.e., released from the targeted construct, before the active agents are released from the conjugates. In some cases, the external linkers are cleaved when the targeted constructs reach a tumor microenvironment to release the conjugates, wherein the conjugates penetrate into the tumor. Not willing to be bound to any theory, the conjugates penetrate deeper and faster into the tumor than the targeted constructs, because the conjugates have a smaller molecular weight once released.

The term “molecular weight,” as used herein, generally refers to the mass or average mass of a material. Its unit may be g/mol, atomic mass unit (amu) or dalton (Da). 1 g/mol=1 amu=1 Da. It can be calculated as the sum of the mass of each constituent atom multiplied by the number of atoms of that element in the chemical formula. For a polymer or oligomer, the molecular weight can refer to the relative average chain length or relative chain mass of the bulk polymer. In practice, the molecular weight of polymers and oligomers can be estimated or characterized in various ways including size exclusion chromatography (SEC), electrophoretic methods, light scattering, mass spectrometry (MS) such as MALDI-TOF, gel permeation chromatography (GPC) or capillary viscometry. SEC is a simple and low cost method to determine molecular weight of polymers and oligomers based on calibration curves. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

### Embodiment 2

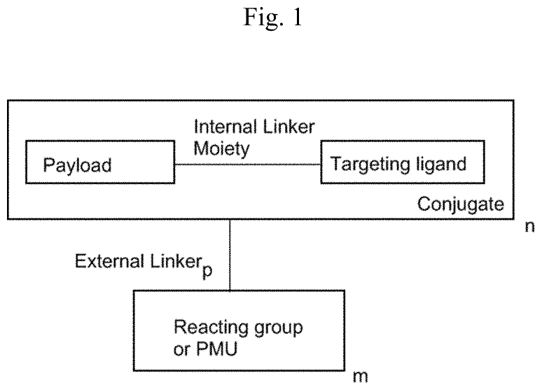

In another embodiment, targeted constructs of the present invention comprise at least one conjugate and at least one reacting group that reacts with a functional group on a protein or an engineered protein or a polymer or derivatives/analogs/mimics thereof: (Conjugate)ₙ-(External linker)ₚ-(Reacting group)ₘ, n≥1, m≥1, and p≥0. The reacting group may be attached to the active agent, the targeting moiety, or the optional internal linker moiety of the conjugate by a covalent bond or an external linker. A non-limiting example of the design of the targeted constructs is shown in FIG. 1.

Alternatively, the internal linker moiety in the conjugate comprises a reacting group. An external linker is not needed in this case.

The reaction between the reacting group and the functional group may happen in vivo after administration or is performed prior to administration. The protein may be a naturally occurring protein such as a serum or plasma protein, or a fragment thereof. Particular examples include thyroxine-binding protein, transthyretin, α1-acid glycoprotein (AAG), transferrin, fibrinogen, albumin, an immunoglobulin, α-2-macroglobulin, a lipoprotein, or fragments thereof. The reaction between the reacting group and the functional group may be reversible.

The external linkers connecting the conjugates and the reacting groups may be cleavable linkers that allow release of the conjugates from the proteins, engineered proteins or polymers. The external linkers connecting the conjugates and the proteins, engineered proteins, or polymers may be less stable than the internal linker moiety in the conjugates connecting the active agent and the targeting moiety. Hence, the conjugates are separated from the proteins, engineered proteins, or polymers, before the active agents are released from the conjugates. In some cases, the external linkers are cleaved when the targeted constructs reach a tumor microenvironment to release the conjugates in an extracellular manner, for instance, by pH-dependent or hypoxia-dependent cleavage that relies upon conditions in the tumor microenvironment, or by extracellular proteases such as matrix metalloproteinases. The conjugates then penetrate into the tumor.

In one example, the functional group is on human serum albumin (HSA or albumin) or its derivative/analog/mimic. Albumin is the most abundant plasma protein (35-50 g/L in human serum) with a molecular weight of 66.5 KDa and an effective diameter of 7.2 nm (Kratz, J. of Controlled Release, vol. 132:171, (2008), the contents of which are incorporated herein by reference in their entirety). Albumin has a half-life of about 19 days. Albumin preferentially accumulates in malignant and inflamed tissues due to leaky capillaries and an absent or defective lymphatic drainage system. Albumin accumulates in tumors such as solid tumors also because albumin is a major energy and nutrition source for tumor growth. The functional group may be the cysteine-34 position of albumin that has an accessible free thiol group. Reacting groups that react with a functional group on albumin or it derivative/analog/mimic may be selected from a disulfide group, a vinylcarbonyl group, a vinyl acetylene group, an aziridine group, an acetylene group or any of the following groups:

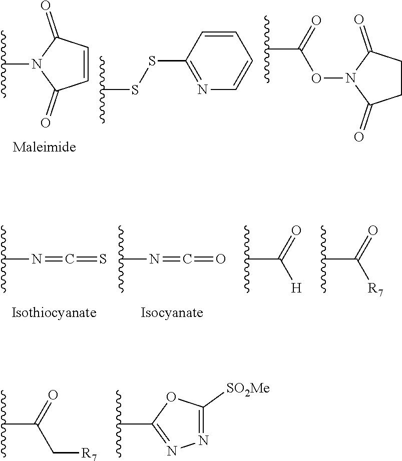

where R⁷ is Cl, Br, F, mesylate, tosylate, O-(4-nitrophenyl), O-pentafluorophenyl, and wherein optionally the activated disulfide group, the vinylcarbonyl group, the vinyl acetylene group, the aziridine group, and the acetylene group may be substituted. The reacting group may also be any protein-binding moiety disclosed in U.S. Pat. No. 9,216,228 to Kratz et al., the contents of which are incorporated herein by reference in their entirety, selected from the group consisting of a maleimide group, a halogenacetamide group, a halogenacetate group, a pyridylthio group, a vinylcarbonyl group, an aziridine group, a disulfide group, a substituted or unsubstituted acetylene group, and a hydroxysuccinimide ester group. In some cases, the reacting group is a disulfide group. The disulfide group undergoes an exchange with a thiol group on a protein or an engineered protein or a polymer or derivatives/analogs/mimics thereof, such as albumin, to form a disulfide between the conjugate and the protein or an engineered protein or a polymer or derivatives/analogs/mimics thereof.

In another example, the functional group is on transthyretin or its derivative/analog/mimic. Transthyretin is a 55 KDa serum protein that has an in vivo half-life of around 48 h. Reacting groups that react with a functional group on transthyretin or it derivative/analog/mimic may be selected from AG10 (structure shown below) or its derivative disclosed by Penchala et al. in Nature Chemical Biology, vol. 11:793, (2015) or formula (I), (II), (III) or (IV) (structures shown below) disclosed in U.S. Pat. No. 5,714,142 to Blaney et al., the contents of each of which are incorporated herein by reference in their entirety. Any transthyretin-selective ligand disclosed on pages 5-8 of Blaney et al. or their derivatives may be used as a reacting group, such as but not limited to, tetraiodothyroacetic acid, 2,4,6-triiodophenol, flufenamic acid, diflunisal, milrinone, EMD 21388.

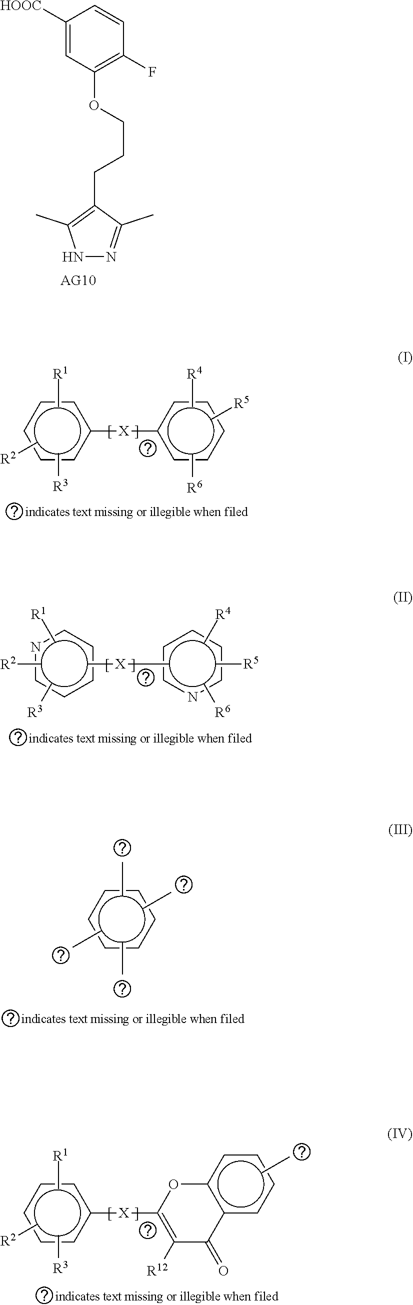

In some cases, the reacting group may be any protein binding moiety may be any protein binding moiety disclosed in U.S. Pat. No. 9,216,228 to Kratz, the contents of which are incorporated herein by reference in their entirety, such as a maleimide group, a halogenacetamide group, a halogenacetate group, a pyridylthio group, a vinylcarbonyl group, an aziridine group, a disulfide group, a substituted or unsubstituted acetylene group, and a hydroxysuccinimide ester group.

### Embodiment 3

In yet another embodiment, targeted constructs of the present invention comprise at least one conjugate and at least one pharmacokinetic modulating unit (PMU in FIG. 1), connected with covalent bonds or optional external linkers: (Conjugate)ₙ-(External linker)ₚ-(Pharmacokinetic modulating unit)ₘ, n≥1, m≥1, and p≥0. The pharmacokinetic modulating unit or pharmacokinetic modulating units have a total molecular weight of at least about 10 KDa, at least about 20 KDa, at least about 30 KDa, at least about 40 KDa or at least about 50 KDa. Generally, the pharmacokinetic modulating unit or pharmacokinetic modulating units have a total molecular weight between about 10 KDa and about 70 KDa. Preferably, the pharmacokinetic modulating unit or pharmacokinetic modulating units have a total molecular weight between about 30 KDa and about 70 KDa, between about 40 KDa and about 70 KDa, between about 50 KDa and about 70 KDa, between about 60 KDa and about 70 KDa. The pharmacokinetic modulating unit may be attached to the active agent, the targeting moiety, or the optional internal linker moiety of the conjugate by the external linker. A non-limiting example of the design of the targeted constructs is shown in FIG. 1.

The external linkers connecting the conjugates and the pharmacokinetic modulating units may be cleavable linkers that allow release of the conjugates. The external linkers connecting the conjugates and the pharmacokinetic modulating units may be less stable than the internal linker moiety in the conjugates connecting the active agent and the targeting moiety. Hence, the conjugates are separated from the pharmacokinetic modulating units, i.e., released from the targeted construct, before the active agents are released from the conjugates. In some cases, the external linkers are cleaved when the targeted constructs reach tumor microenvironment to release the conjugates in an extracellular manner, for instance, by pH-dependent or hypoxia-dependent cleavage that relies upon conditions in the tumor microenvironment, or by extracellular proteases such as matrix metalloproteinases. The conjugates then penetrate into the tumor.

The pharmacokinetic modulating unit may be a natural or synthetic protein or fragment thereof. For example, it may be a serum protein such as thyroxine-binding protein, transthyretin, α1-acid glycoprotein (AAG), transferrin, fibrinogen, albumin, an immunoglobulin, α-2-macroglobulin, a lipoprotein, or fragments thereof. The pharmacokinetic modulating unit may also be a natural or synthetic polymer, such as polysialic acid unit, a hydroxyethyl starch (HES) unit, or a polyethylene glycol (PEG) unit. Further, the pharmacokinetic modulating unit may be a particle, such as dendrimers, inorganic nanoparticles, organic nanoparticles, and liposomes.

The in vitro biological activity of the conjugate may be reduced when the conjugate is attached to pharmacokinetic modulating unit, because the pharmacokinetic modulating unit may be a steric hindrance and influence the function of the active agent or the targeting moiety. However, the in vivo pharmacological effects are usually enhanced. Targeted delivery of the active agent is increased because of the EPR effect of the targeted constructs comprising at least one conjugate and at least one pharmacokinetic modulating unit, and the active targeting of the targeting moiety in the conjugate. Moreover, the pharmacokinetic modulating unit increases molecular weight, extends the half-life, and shields the active agent from the environment. Cleavage of the external linker that connects the pharmacokinetic modulating unit to the conjugate after delivery to the tissue of interest then unveils the more active free conjugate. The pharmacokinetic modulating unit may also reduce immunogenicity of the active agent.

In one example, the pharmacokinetic modulating unit is a PEG unit. The PEG unit may comprise PEG chains (i.e., linear PEGs) or branched PEGs such as forked PEGs, multiarm PEGs or comb-shaped PEGs. For example, the PEG unit may be a triple branched PEG molecule disclosed in US Pat. Appln. No. 20070184015 to Hahn, a cross-linked network of linear PEGs disclosed in U.S. Pat. No. 4,894,238 to Embry et al., or a degradable PEG hydrogel disclosed in WO1999022770 to Harris, the contents of each of other are incorporated herein by reference in their entirety.

In another example, the pharmacokinetic modulating unit is a dendrimer or its analog. Dendrimers refer to any monodisperse, highly symmetric, branched, macromolecule compound. Branching reactions are performed onto a core molecule to generate dendrimers. Dendrimers may have functional groups on the surface, also called terminal groups, which may be later used to link to other molecules or to customize for a given application.

Dendrimers such as polyamidoamine dendrimer, polypropylene dendrimer, polyethyleneimine dendrimer, carbohydrate based dendrimer, peptide based dendrimer, glycopeptide dendrimer, metal containing dendrimer, poly aryl amine dendrimer, polyamide dendrimer, poly (alkyl amine) dendrimer, polyamido alcohol dendrimer, cyano dendrimer, polyether dendrimer, polythioether dendrimer, polysiloxane dendrimer, dendritic aryl ester, perchlorinated dendrimer, catylitic centre containing dendrimer, silicon containing dendrimer, phosphorus containing dendrimer, hydrocarbon dendrimer, or any molecule possessing dendritic framework of controlled architecture may be used as a pharmacokinetics modulating unit. Non-limiting examples include polyamidoamine (PAMAM) dendrimers disclosed in U.S. Pat. Nos. 4,507,466, 4,558,120, 4,568,737 and 4,587,329, lysine based polylysine dendrimer discussed in U.S. Pat. Nos. 4,289,872 and 4,410,688, NH₂-terminated PAMAM dendrimer (EDA core) generation 4, aliphatic hydroxyl terminated PAMAM dendrimer (EDA core) generation 4 disclosed in US 20030180250 Chauhan et al., a silane- or carbosilane-based, periphery-modified dendrimer disclosed in U.S. Pat. No. 6,184,313 to Roovers et al., or dendritic polymers or copolymers composed of building blocks that are biocompatible or are natural metabolites in vivo including but not limited to glycerol, lactic acid; glycolic acid, glycerol, amino acids, caproic acid, ribose, glucose, succinic acid, malic acid, amino acids, peptides, synthetic peptide analogs, poly(ethylene glycol), poly(hydroxyacids) [e.g., PGA. PLA] as disclosed in 20040086479 to Grinstaff et al., the contents of each of which are incorporated herein by reference in their entirety.

In yet another example, the pharmacokinetic modulating unit is a polymer. In some embodiments, the polymer may be a polymeric scaffold, such as a polymer comprises poly(1-hydroxymethylethylene hydroxymethyl-formal) (PHF) having a molecular weight ranging from about 20 kDa to about 150 kDa as disclosed in WO2012171020 (Mersana) to Yurkovetskiy et al., the contents of which are incorporated herein by reference in their entirety. For example, the polymer scaffold may comprise formula (Ia) or formula (Ib) in WO2012171020. The linker connecting the polymer scaffold to the conjugate, the targeting moiety, and/or the therapeutic agent may be LD or LP in WO2012171020.

In some embodiments, the polymer may be a polyal such as a polyacetal or polyketal disclosed in WO2010138719 to Yurkovetskiy et al., the contents of which are incorporated herein by reference in their entirety. The linker between the polyal and the conjugate may be a rate-releasing linker in formula (I) or (II) in WO20100138719.

In one embodiments, the polymer may comprise a polymer polyacetal backbone of poly[1-hydroxymethylethylene hydroxymethyl-formal] (PHF) disclosed in WO2010068759 to ROLKE et al., the contents of which are incorporated herein by reference in their entirety.

In another embodiment, the polymer may be a water soluble polyal having at least one acetal oxygen atom or ketal oxygen atom in each monomer unit positioned within the main chain of the polymer as disclosed in WO2009073445 to AKULLIAN et al., the contents of which are incorporated herein by reference in their entirety.

In one embodiment, the polymer may be a modified polymer is provided herein, the modified polymer having the formula (I) in WO2011120053 to YURKOVETSKIY et al., the contents of which are incorporated herein by reference in their entirety.

In another embodiment, the polymer may be a polymeric carrier disclosed in WO2014160360 to YURKOVETSKIY et al., the contents of which are incorporated herein by reference in their entirety, such as a polyacetal, e.g., a poly(1-hydroxymethyl ethylene hydroxymethyl-formal) (PHF) having a molecular weight (i.e., MW of the unmodified PHF) ranging from about 2 kDa to about 40 kDa (e.g., about 6-20 kDa or about 8-15 kDa).

In another embodiment, the polymer may be a polymeric scaffold with Formula (Ia), (Ic), (Id), or (If) disclosed in WO2015195917 to BODYAK et al., the contents of which are incorporated herein by reference in their entirety.

In another embodiment, the polymer may be a poly(1-hydroxymethylethylene hydroxymethyl-formal) (PHF) polymer scaffold including one or more maleimide groups, such as the PHF polymer scaffold in Formula (Ie) of WO2015195925 to BODYAK et al., the contents of which are incorporated herein by reference in their entirety, wherein the PHF polymer scaffold has a molecular weight ranging from about 2 kDa to about 40 kDa

In another embodiment, the polymer may be a polymer scaffold comprising a linear, branched or cyclic polymer having one or more —OH groups connected to the polymer, wherein the polymer is a hydroxyl polymer having a molecular weight ranging from 2 kDa to 300 kDa and the polymer is not a polyacetal or a polyketal, as disclosed in WO2014093640 to YURKOVETSKIY, the contents of which are incorporated herein by reference in their entirety. The polymer may comprise the structure of formula (Ibb), (Icc), or (Idd).

In another embodiment, the polymer may be a polymeric scaffold comprising poly(1-hydroxymethylethylene hydroxymethyl-formal) (PHF) having a molecular weight ranging from about 2 kDa to about 40 kDa and comprising a maleimide group, such as Formula (Id), (Ia), (Ie), (Ib), (Ic), (A), or (B) in WO2015054659 to YURKOVETSKIY et al., the contents of which are incorporated herein by reference in their entirety.

In another embodiment, the polymer may be a terminally modified polymer with a terminal functional group, wherein the polymer is a polyacetal or polyketal with a molecular weight between about 0.5 and about 150 kDa. The terminal functional group may be selected any of functional group (1)-(33) in WO2014008375, the contents of which are incorporated herein by reference in their entirety.

In another embodiment, the polymer may comprise covalently bound subunits, such as subunits L, K, and M in WO2013096901, or subunits m, m1, m2, m3, and m4 in WO2014093394, the contents of which are incorporated herein by reference in their entirety.

Conjugates

The targeted constructs of the present invention comprise at least one conjugate. The conjugates can be a conjugate between a single active agent and a single targeting moiety, e.g. a conjugate having the structure X-Y-Z where X is the targeting moiety, Y is the optional internal linker moiety, and Z is the active agent.

In some embodiments the conjugate contains more than one targeting moiety, more than one internal linker moiety, more than one active agent, or any combination thereof. The conjugate can have any number of targeting moieties, internal linker moieties, and active agents. The conjugate can have the structure X-Y-Z-Y-X, (X-Y)ₙ-Z, Xₙ-Y-Z, X-(Y-Z)ₙ, X-Y-Zₙ, (X-Y-Z)ₙ, (X-Y-Z-Y)ₙ-Z where X is a targeting moiety, Y is an internal linker moiety, Z is an active agent, and n is an integer between 1 and 50, between 2 and 20, for example, between 1 and 5. Each occurrence of X, Y, and Z can be the same or different, e.g. the conjugate can contain more than one type of targeting moiety, more than one type of internal linker moiety, and/or more than one type of active agent.

The conjugate can contain more than one targeting moiety attached to a single active agent. For example, the conjugate can include an active agent with multiple targeting moieties each attached via a different internal linker moiety. The conjugate can have the structure X-Y-Z-Y-X where each X is a targeting moiety that may be the same or different, each Y is an internal linker moiety that may be the same or different, and Z is the active agent.

The conjugate can contain more than one active agent attached to a single targeting moiety. For example the conjugate can include a targeting moiety with multiple active agents each attached via a different internal linker moiety. The conjugate can have the structure Z-Y-X-Y-Z where X is the targeting moiety, each Y is an internal linker moiety that may be the same or different, and each Z is an active agent that may be the same or different.

The conjugate may comprise pendent or terminal functional groups that allow further modification or conjugation. The pendent or terminal functional groups may be protected with any suitable protecting groups.

The conjugate is present in an amount between about 0.05% and 90% (w/w), between about 1% and 80%, or between about 5% and 50% in the targeted constructs of the present invention based on the weight of the targeted constructs.

A. Active Agents

A conjugate as described herein contains at least one active agent (a first active agent). The conjugate can contain more than one active agent, that can be the same or different from the first active agent. The active agent can be a therapeutic, prophylactic, diagnostic, or nutritional agent. A variety of active agents are known in the art and may be used in the conjugates described herein. The active agent can be a protein or peptide, small molecule, nucleic acid or nucleic acid molecule, lipid, sugar, glycolipid, glycoprotein, lipoprotein, or combination thereof. In some embodiments, the active agent is an antigen, an adjuvant, radioactive, an imaging agent (e.g., a fluorescent moiety) or a polynucleotide. In some embodiments the active agent is an organometallic compound.

In certain embodiments, the active agent of the conjugate comprises a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of active agent(s) of the conjugate may also be expressed in terms of proportion to the targeting ligand(s). For example, the present teachings provide a ratio of active agent to ligand of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

In some embodiments, the active agent can be a cancer therapeutic. Cancer therapeutics include, for example, death receptor agonists such as the TNF-related apoptosis-inducing ligand (TRAIL) or Fas ligand or any ligand or antibody that binds or activates a death receptor or otherwise induces apoptosis. Suitable death receptors include, but are not limited to, TNFR1, Fas, DR3, DR4, DR5, DR6, LTβR and combinations thereof.

Cancer therapeutics such as chemotherapeutic agents, cytokines, chemokines, and radiation therapy agents can be used as active agents. Chemotherapeutic agents include, for example, alkylating agents, antimetabolites, anthracyclines, plant alkaloids, topoisomerase inhibitors, and other antitumor agents. Such agents typically affect cell division or DNA synthesis and function. Additional examples of therapeutics that can be used as active agents include monoclonal antibodies and the tyrosine kinase inhibitors e.g. imatinib mesylate, which directly targets a molecular abnormality in certain types of cancer (e.g., chronic myelogenous leukemia, gastrointestinal stromal tumors).

Chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab, cetuximab, and rituximab, bevacizumab, and combinations thereof. Any of these may be used as an active agent in a conjugate.

In certain embodiments, the active agent is a small molecule having a molecular weight preferably <about 5 kDa, more preferably <about 4 kDa, more preferably about 3 kDa, most preferably <about 1.5 kDa or <about 1 kDa.

The small molecule active agents used in this invention (e.g. antiproliferative (cytotoxic and cytostatic) agents) include cytotoxic compounds (e.g., broad spectrum), angiogenesis inhibitors, cell cycle progression inhibitors, PBK/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, RNA polymerase inhibitors and proteasome inhibitors. The small molecule active agents in some embodiments the active agent is an analog, derivative, prodrug, or pharmaceutically acceptable salt thereof.

Broad spectrum cytotoxins include, but are not limited to, DNA-binding or alkylating drugs, microtubule stabilizing and destabilizing agents, platinum compounds, and topoisomerase I or II inhibitors.

Exemplary DNA-binding or alkylating drugs include, CC-1065 and its analogs, anthracyclines (doxorubicin, epirubicin, idarubicin, daunorubicin) and its analogs, alkylating agents, such as calicheamicins, dactinomycines, mitromycines, pyrrolobenzodiazepines, and the like.

Exemplary doxorubicin analogs include nemorubicin metabolite or analog drug moiety disclosed in US 20140227299 to Cohen et al., the contents of which are incorporated herein by reference in their

Exemplary CC-1065 analogs include duocarmycin SA, duocarmycin CI, duocarmycin C2, duocarmycin B2, DU-86, KW-2189, bizelesin, seco-adozelesin, and those described in U.S. Pat. Nos. 5,475,092; 5,595,499; 5,846,545; 6,534,660; 6,586,618; 6,756,397 and 7,049,316. Doxorubicin and its analogs include PNU-159682 and those described in U.S. Pat. No.6,630,579 and nemorubicin metabolite or analog drugs disclosed in US 20140227299 to Cohen et al., the contents of which are incorporated herein by reference in their entirety.

Calicheamicins include those described in U.S. Pat. Nos. 5,714,586 and 5,739,116. Duocarmycins include those described in U.S. Pat. Nos.5,070,092; 5,101,038; 5,187,186; 6,548,530; 6,660,742; and 7,553,816 B2; and Li et al., Tet Letts., 50:2932-2935 (2009). Pyrrolobenzodiazepines include SG2057 and those described in Denny, Exp. Opin. Ther. Patents., 10(4):459-474 (2000), Anti-Cancer Agents in Medicinal Chemistry, 2009, 9, 1-31; WO 2011/130613 A1; EP 2 789 622 A1; Blood 2013, 122, 1455; J. Antimicrob. Chemother. 2012, 67, 1683-1696; Cancer Res. 2004, 64, 6693-6699; WO 2013041606; U.S. Pat. No. 8,481,042; WO 2013177481; WO 2011130613; WO2011130598

Exemplary microtubule stabilizing and destabilizing agents include taxane compounds, such as paclitaxel, docetaxel, cabazitaxel; maytansinoids, auristatins and analogs thereof, tubulysin A and B derivatives, vinca alkaloid derivatives, epothilones, PM060184 and cryptophycins.

Exemplary maytansinoids or maytansinoid analogs include maytansinol and maytansinol analogs, maytansine or DM-1 and DM-4 are those described in U.S. Pat. Nos. 5,208,020; 5,416,064; 6,333.410; 6,441,163; 6,716,821; RE39,151 and 7,276,497. In certain embodiments, the cytotoxic agent is a maytansinoid, another group of anti-tubulin agents (ImmunoGen, Inc.; see also Chari et al., 1992, Cancer Res. 52: 127-131), maytansinoids or maytansinoid analogs. Examples of suitable maytansinoids include maytansinol and maytansinol analogs. Suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Exemplary auristatins include auristatin E (also known as a derivative of dolastatin-10), auristatin EB (AEB), auristatin EFP (AEFP), monomethyl auristatin E (MMAE), monomethyl auristatin F (MMAF), auristatin F and dolastatin. Suitable auristatins are also described in U.S. Publication Nos. 2003/0083263, 2011/0020343, and 2011/0070248; PCT Application Publication Nos. WO 09/117531, WO 2005/081711, WO 04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902;5,554,725; 5,530,097; 5,521,284; 5,504,191; 5,410,024; 5,138,036; 5,076,973; 4,986,988; 4,978,744; 4,879,278; 4,816,444; and 4,486,414, the disclosures of which are incorporated herein by reference in their entirety.

Exemplary tubulysin compounds include compounds described in U.S. Pat. Nos. 7,816,377; 7,776,814; 7,754,885; U.S. Publication Nos. 2011/0021568; 2010/004784; 2010/0048490; 2010/00240701; 2008/0176958; and PCT Application Nos. WO 98/13375; WO 2004/005269; WO 2008/138561; WO 2009/002993; WO 2009/055562; WO 2009/012958; WO 2009/026177; WO 2009/134279; WO 2010/033733; WO 2010/034724; WO 2011/017249; WO 2011/057805; the disclosures of which are incorporated by reference herein in their entirety.

Exemplary vinca alkaloids include vincristine, vinblastine, vindesine, and navelbine (vinorelbine). Suitable Vinca alkaloids that can be used in the present invention are also disclosed in U.S. Publication Nos. 2002/0103136 and 2010/0305149, and in U.S. Pat. No. 7,303,749 B1, the disclosures of which are incorporated herein by reference in their entirety.

Exemplary epothilone compounds include epothilone A, B, C, D, E and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO 97/19086; WO 98/08849; WO 98/22461; WO 98/25929; WO 98/38192; WO 99/01124; WO 99/02514; WO 99/03848; WO 99/07692; WO 99/27890; and WO 99/28324; the disclosures of which are incorporated herein by reference in their entirety.

Exemplary cryptophycin compounds are described in U.S. Pat. Nos. 6,680,311 and 6,747,021, the disclosures of which are incorporated herein by reference in their entirety.

Exemplary platinum compounds include cisplatin (PLATINOL®), carboplatin (PARAPLATIN®), oxaliplatin (ELOX ATINE®), iproplatin, ormaplatin, and tetraplatin.

Exemplary topoisomerase I inhibitors include camptothecin, camptothecin, derivatives, camptothecin analogs and non-natural camptothecins, such as, for example, CPT-11 (irinotecan), SN-38, topotecan, 9-aminocamptothecin, rubitecan, gimatecan, karenitecin, silatecan, lurtotecan, exatecan, diflomotecan, belotecan, lurtotecan and S39625. Other camptothecin compounds that can be used in the present invention include those described in, for example, J. Med. Chem., 29:2358-2363 (1986); J. Med. Chem., 23:554 (1980); J. Med. Chem., 30: 1774 (1987).

Exemplary topoisomerase II inhibitors include azonafide and etoposide.

Additional agents acting on DNA include Lurbinectedin (PM01183), Trabectedin (also known as ecteinascidin 743 or ET-743) and analogs as described in WO 200107711, WO 2003014127.

Angiogenesis inhibitors include, but are not limited to, MetAP2 inhibitors.

Exemplary MetAP2 inhibitors include fumagillol analogs, meaning any compound that includes the fumagillin core structure, including fumagillamine, that inhibits the ability of MetAP-2 to remove NH₂-terminal methionines from proteins as described in Rodeschini et al., J. Org. Chem., 69, 357-373, 2004 and Liu, et al., Science 282, 1324-1327, 1998. Non limiting examples of “fumagillol analogs” are disclosed in J. Org. Chem., 69, 357, 2004; J. Org. Chem., 70, 6870, 2005; European Patent Application 0 354 787; J. Med. Chem., 49, 5645, 2006; Bioorg. Med. Chem., 11, 5051, 2003; Bioorg. Med. Chem., 14, 91, 2004; Tet. Lett. 40, 4797, 1999; WO99/61432; U.S. Pat. Nos. 6,603,812; 5,789,405; 5,767,293; 6,566,541; and 6,207,704.

Exemplary cell cycle progression inhibitors include CDK inhibitors such as BMS-387032 and PD0332991; Rho-kinase inhibitors such as GSK429286; checkpoint kinase inhibitors such as AZD7762; aurora kinase inhibitors such as AZD1152, MLN8054 and MLN8237; PLK inhibitors such as BI 2536, BI6727 (Volasertib), GSK461364, ON-01910 (Estybon); and KSP inhibitors such as SB 743921, SB 715992 (ispinesib), MK-0731, AZD8477, AZ3146 and ARRY-520.

Exemplary PI3K/m-TOR/AKT signaling pathway inhibitors include phosphoinositide 3-kinase (PI3K) inhibitors, GSK-3 inhibitors, ATM inhibitors, DNA-PK inhibitors and PDK-1 inhibitors.

Exemplary PI3 kinase inhibitors are disclosed in U.S. Pat. No. 6,608,053, and include BEZ235, BGT226, BKM120, CAL101, CAL263, demethoxyviridin, GDC-0941, GSK615, IC87114, LY294002, Palomid 529, perifosine, PF-04691502, PX-866, SAR245408, SAR245409, SF1126, Wortmannin, XL147, XL765, GSK2126458 (Omipalisib), GDC-0326, GDC-0032 (Taselisib, RG7604), PF-05212384 (Gedatolisib, PKI-587), BAY 80-6946 (copanlisib), PF-04691502, PF-04989216, PI-103, PKI-402 VS-5584 (SB2343), GDC-0941, NVP-BEZ235 (Dactoslisib), BGT226, NVP-BKM120 (Buparlisib), NVP-BYL719 (alpelisib), GSK2636771, AMG-319, GSK2269557, PQR309, PWT143, TGR-1202 (RP5264), PX-866, GDC-0980 (apitolisib), AZD8835, MLN1117, DS-7423, ZSTK474, CUDC-907, IPI-145 (INK-1197, Duvelisib), AZD8186, XL147 (SAR245408), XL765 (SAR245409), CAL-101 (Idelalisib, GS-1101), GS-9820 (Acalisib) and KA2237.

Exemplary AKT inhibitors include, but are not limited to, AT7867, MK-2206, Perifosine, GSK690693, Ipatasertib, AZD5363, TIC10, Afuresertib, SC79, AT13148, PHT-427, A-674563, and CCT128930.

Exemplary MAPK signaling pathway inhibitors include MEK, Ras, JNK, B-Raf and p38 MAPK inhibitors.

Exemplary MEK inhibitors are disclosed in U.S. Pat. No. 7,517,994 and include GDC-0973, GSK1120212, MSC1936369B, AS703026, R05126766 and R04987655, PD0325901, AZD6244, AZD 8330 and GDC-0973.

Exemplary B-raf inhibitors include CDC-0879, PLX-4032, and SB590885.

Exemplary B p38 MAPK inhibitors include BIRB 796, LY2228820 and SB202190

Receptor tyrosine kinases (RTK) are cell surface receptors which are often associated with signaling pathways stimulating uncontrolled proliferation of cancer cells and neoangiogenesis. Many RTKs, which over express or have mutations leading to constitutive activation of the receptor, have been identified, including, but not limited to, VEGFR, EGFR, FGFR, PDGFR, EphR and RET receptor family receptors. Exemplary RTK specific targets include ErbB2, FLT-3, c-Kit, c-Met, and HIF.

Exemplary inhibitors of ErbB2 receptor (EGFR family) include but not limited to AEE788 (NVP-AEE 788), BIBW2992 (Afatinib), Lapatinib, Erlotinib (Tarceva), and Gefitinib (Iressa).

Exemplary RTK inhibitors targeting more than one signaling pathway (multitargeted kinase inhibitors) include AP24534 (Ponatinib) that targets FGFR, FLT-3, VEGFR-PDGFR and Bcr-Abl receptors; ABT-869 (Linifanib) that targets FLT-3 and VEGFR-PDGFR receptors; AZD2171 that targets VEGFR-PDGFR, Flt-1 and VEGF receptors; CHR-258 (Dovitinib) that targets VEGFR-PDGFR, FGFR, Flt-3, and c-Kit receptors.

Exemplary kinase inhibtiors include inhibitors of the kinases ATM, ATR, CHK1, CHK2, WEE1, and RSK.

Exemplary protein chaperon inhibitors include HSP90 inhibitors. Exemplary HSP90 inhibitors include 17AAG derivatives, BIIB021, BIIB028, SNX-5422, NVP-AUY-922, and KW-2478.

Exemplary HDAC inhibitors include Belinostat (PXD101), CUDC-101, Doxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A, and Vorinostat (SAHA).

Exemplary PARP inhibitors include iniparib (BSI 201), olaparib (AZD-2281), ABT-888 (Veliparib), AG014699, CEP 9722, MK 4827, KU-0059436 (AZD2281), LT-673, 3-aminobenzamide, A-966492, and AZD2461

Exemplary Wnt/Hedgehog signaling pathway inhibitors include vismodegib (RG3616/GDC-0449), cyclopamine (11-deoxojervine) (Hedgehog pathway inhibitors), and XAV-939 (Wnt pathway inhibitor).

Exemplary RNA polymerase inhibitors include amatoxins. Exemplary amatoxins include a-amanitins, β-amanitins, γ-amanitins, ε-amanitins, amanullin, amanullic acid, amaninamide, amanin, and proamanullin.

Exemplary proteasome inhibitors include bortezomib, carfilzomib, ONX 0912, CEP-18770, and MLN9708.

In one embodiment, the drug of the invention is a non-natural camptothecin compound, vinca alkaloid, kinase inhibitor (e.g., PI3 kinase inhibitor (GDC-0941 and PI-103)), MEK inhibitor, KSP inhibitor, RNA polymerse inhibitor, PARP inhibitor, docetaxel, paclitaxel, doxorubicin, duocarmycin, tubulysin, auristatin or a platinum compound. In specific embodiments, the drug is a derivative of SN-38, vindesine, vinblastine, PI-103, AZD 8330, auristatin E, auristatin F, a duocarmycin compound, tubulysin compound, or ARRY-520.

In another embodiment, the drug used in the invention is a combination of two or more drugs, such as, for example, PI3 kinases and MEK inhibitors; broad spectrum cytotoxic compounds and platinum compounds; PARP inhibitors and platinum compounds; broad spectrum cytotoxic compounds and PARP inhibitors.

The active agent can be a cancer therapeutic. The cancer therapeutics may include death receptor agonists such as the TNF-related apoptosis-inducing ligand (TRAIL) or Fas ligand or any ligand or antibody that binds or activates a death receptor or otherwise induces apoptosis. Suitable death receptors include, but are not limited to, TNFR1, Fas, DR3, DR4, DR5, DR6, LTβR and combinations thereof.

The active agent can be a DNA minor groove binders such as lurbectidin and trabectidin.

The active agent can be E3 ubiquitin ligase inhibitors, adeubiquitinase inhibitors or an NFkB pathway inhibitor.

The active agent can be a phopsphatase inhibitors including inhibitors of PTP1B, SHP2, LYP, FAP-1, CD45, STEP, MKP-1, PRL, LMWPTP or CDC25.

The active agent can be an inhibitor of tumor metabolism, such as an inhibitor of GAPDH, GLUT1, HK II, PFK, GAPDH, PK, LDH or MCTs

The active agent can target epigenetic targets including EZH2, MLL, DOT1-like protein (DOT1L), bromodomain-containing protein 4 (BRD4), BRD2, BRD3, NUT, ATAD2, or SMYD2.

The active agent can target the body's immune system to help fight cancer, including moecules targeting IDO1, IDO2, TDO, CD39, CD73, A2A antagonists, STING activators, TLR agonists (TLR 1-13), ALK5, CBP/EP300 bromodomain, ARG1, ARG2, iNOS, PDE5, P2X7, P2Y11, COX2, EP2 Receptor, or EP4 receptor.

The active agent can target Bcl-2, IAP, or fatty acid synthase.

In some embodiments, the active agent can be 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cabazitaxel, cactinomycin, calcipotriol, calphostin C, calusterone, camptothecin, camptothecin derivatives, canarypox IL-2, capecitabine, caracemide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castano spermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, larotaxel, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, maytansinoid, mertansine (DM1), mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum(IV) complexes, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxy ethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone Bl, ruboxyl, safingol, safingol hydrochloride, saintopin, sarcnu, sarcophytol A, sargramostim, SDI 1 mimetics, semustine, senescence derived inhibitor 1, sense oligonucleotides, siRNA, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

The active agent can be an inorganic or organometallic compound containing one or more metal centers. In some examples, the compound contains one metal center. The active agent can be, for example, a platinum compound, a ruthenium compound (e.g., trans-[RuCl₂ (DMSO)₄], or trans-[RuCl₄(imidazole)₂, etc.), cobalt compound, copper compound, or iron compounds.

In some embodiments, the active agent is a small molecule. In some embodiments, the active agent is a small molecule cytotoxin. In one embodiment, the active agent is cabazitaxel, or an analog, derivative, prodrug, or pharmaceutically acceptable salt thereof. In another embodiment, the active agent is mertansine (DM1) or DM4, or an analog, derivative, prodrug, or pharmaceutically acceptable salt thereof. DM1 or DM4 inhibits the assembly of microtubules by binding to tubulin. Structure of DM1 is shown below:

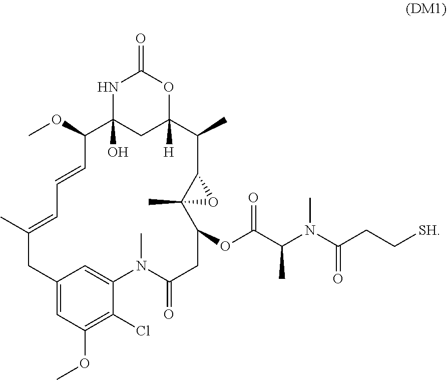

In some embodiments, the active agent Z is Monomethyl auristatin E (MMAE), or an analog, derivative, prodrug, or pharmaceutically acceptable salt thereof. Structure of MIVIAE is shown below:

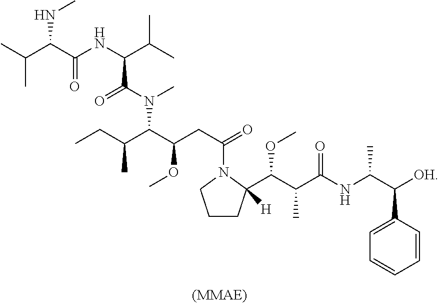 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In some embodiments, the active agent Z is a sequence-selective DNA minor-groove binding crosslinking agent. For example, Z may be pyrrolobenzodiazepine (PBD), a PBD dimer, or an analog, derivative, prodrug, or pharmaceutically acceptable salt thereof. Structures of PBD and PBD dimer are shown below:

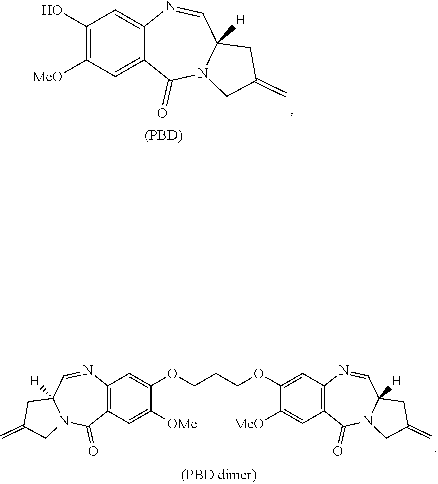

In some embodiments, the active agent Z is a topoisomerase I inhibitor, such as camptothecin, irinotecan, SN-38, or an analog, derivative, prodrug, or pharmaceutically acceptable salt thereof.

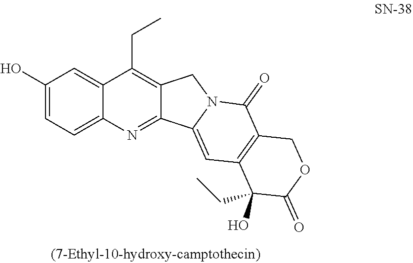 [SMILES: CCc1c2c(nc3ccc(O)cc13)-c1cc3c(c(=O)n1C2)COC(=O)[C@]3(O)CC]

Any cytotoxic moiety disclosed in WO2013158644, WO2015038649, WO2015066053, WO2015116774, WO2015134464, WO2015143004, WO2015184246, the contents of each of which are incorporated herein by reference in their entirety, such as bendamustine, VDA, doxorubicin, pemetrexed, vorinostat, lenalidomide, docetaxel, 17-AAG, 5-FU, abiraterone, crizotinib, KW-2189, BUMB2, DC1, CC-1065, adozelesin, or derivatives/analogs thereof, may be used as an active agent in conjugates of the present invention.

B. Targeting Moieties

The conjugates contain one or more targeting moieties and/or targeting ligands. Targeting ligands or moieties can be peptides, antibody mimetics, nucleic acids (e.g., aptamers), polypeptides (e.g., antibodies), glycoproteins, small molecules, carbohydrates, or lipids. The targeting moiety, X, can be a peptide such as somatostatin, octreotide, LHRH, an EGFR-binding peptide, RGD-containing peptides, a protein scaffold such as a fibronectin domain, an aptide or bipodal peptide, a single domain antibody, a stable scFv, or a bispecific T-cell engagers, nucleic acid (e.g., aptamer), polypeptide (e.g., antibody or its fragment), glycoprotein, small molecule, carbohydrate, or lipid. The targeting moiety, X can be an aptamer being either RNA or DNA or an artificial nucleic acid; small molecules; carbohydrates such as mannose, galactose and arabinose; vitamins such as ascorbic acid, niacin, pantothenic acid, carnitine, inositol, pyridoxal, lipoic acid, folic acid (folate), riboflavin, biotin, vitamin B12, vitamin A, E, and K; a protein or peptide that binds to a cell-surface receptor such as a receptor for thrombospondin, tumor necrosis factors (TNF), annexin V, interferons, cytokines, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

In some embodiments, the targeting moiety is a protein scaffold. The protein scaffold may be an antibody-derived protein scaffold. Non-limiting examples include single domain antibody (dAbs), nanobody, single-chain variable fragment (scFv), antigen-binding fragment (Fab), Avibody, minibody, CH2D domain, Fcab, and bispecific T-cell engager (BiTE) molecules. In some embodiments, scFv is a stable scFv, wherein the scFv has hyperstable properties. In some embodiments, the nanobody may be derived from the single variable domain (VHH) of camelidae antibody.

In some embodiments, the protein scaffold may be a nonantibody-derived protein scaffold, wherein the protein scaffold is based on nonantibody binding proteins. The protein scaffold may be based on enginnered Kunitz domains of human serine protease inhibitors (e.g., LAC1-D1), DARPins (designed ankyrin repeat domains), avimers created from multimerized low-density lipoprotein receptor class A (LDLR-A), anticalins derived from lipocalins, knottins constructed from cysteine-rich knottin peptides, affibodies that are based on the Z-domain of staphylococcal protein A, adnectins or monobodies and pronectins based on the 10^{th} or 14^{th} extracellular domain of human fibronectin III, Fynomers derived from SH3 domains of human Fyn tyrosine kinase, or nanofitins (formerly Affitins) derived from the DNA binding protein Sac7d.

In some embodiments, the protein scaffold may be any protein scaffold disclosed in Mintz and Crea, BioProcess, vol. 11(2):40-48 (2013), the contents of which are incorporated herein by reference in their entirety. Any of the protein scaffolds disclosed in Tables 2-4 of Mintz and Crea may be used as a targeting moiety of the conjugate of the invention.

In some embodiments, the protein scaffold may be based on a fibronectin domain. In some embodiments, the protein scaffold may be based on fibronectin type III (FN3) repeat protein. In some embodiments, the protein scaffold may be based on a consensus sequence of multiple FN3 domains from human Tenascin-C (hereinafter “Tenascin”). Any protein scaffold based on a fibronectin domain disclosed in U.S. Pat. No. 8,569,227 to Jacobs et al., the contents of which are incorporated herein by reference in their entirety, may be used as a targeting moiety of the conjugate of the invention.

In some embodiments, the targeting moiety or targeting ligand may be any molecule that can bind to luteinizing-hormone-releasing hormone receptor (LHRHR). Such targeting ligands can be peptides, antibody mimetics, nucleic acids (e.g., aptamers), polypeptides (e.g., antibodies), glycoproteins, small molecules, carbohydrates, or lipids. In some embodiments, the targeting moiety is LHRH or a LHRH analog.

Luteinizing-hormone-releasing hormone (LHRH), also known as gonadotropin-releasing hormone (GnRH) controls the pituitary release of gonadotropins (LH and FSH) that stimulate the synthesis of sex steroids in the gonads. LHRH is a 10-amino acid peptide that belongs to the gonadotropin-releasing hormone class. Signaling by LHRH is involved in the first step of the hypothalamic-pituitary-gonadal axis. An approach in the treatment of hormone-sensitive tumors directed to the use of agonists and antagonists of LHRH (A. V. Schally and A. M. Comaru-Schally. Sem. Endocrinol., 5 389-398, 1987) has been reported. Some LHRH agonists, when substituted in position 6, 10, or both are much more active than LHRH and also possess prolonged activity. Some LHRH agonists are approved for clinical use, e.g., Leuprolide, triptorelin, nafarelin and goserelin.

Some human tumors are hormone dependent or hormone-responsive and contain hormone receptors. Certain of these tumors are dependent on or responsive to sex hormones or growth factors, or have components that are dependent or responsive to such hormones. Mammary carcinomas contain estrogen, progesterone, glucocorticoid, LHRH, EGF IGF-I and somatostatin receptors. Peptide hormone receptors have been detected in acute leukaemia, prostate-, breast-, pancreatic, ovarian-, endometrial cancer, colon cancer and brain tumors (M. N. Pollak, et al., Cancer Lett. 38 223-230 1987; F. Pekonen, et al., Cancer Res., 48 1343-1347, 1988; M. Fekete, et al., J Clin. Lab. Anal. 3 137-147, 1989; G. Emons, et al., Eur. J. Cancer Oncol., 25215-221 1989). It has been found (M. Fekete, et al., Endocrinology. 124 946-955. 1989; M Fekete, et al. Pancreas 4521-528, 1989) that both agonistic and antagonistic analog of LHRH bind to human breast cancer cell membranes, and also to the cell membranes of pancreatic cancer. It has been demonstrated that biologically active peptides such a melanotropin (MSH), epidermal growth factor, insulin and agonistic and antagonist analogs of LHRH (L Jennes, et. al., Peptides 5 215-220, 1984) are internalized b their target cells by endocytosis.

The conjugates of the invention can employ any of the large number of known molecules that recognize the LHRH receptor, such as known LHRH receptor agonists and antagonists. In some embodiments, the LHRH analog portion of the conjugate contains between 8 and 18 amino acids.

Examples of LHRH binding molecules useful in the present invention are described herein. Further non-limiting examples are analogs of pyroGlu-His-Trp-Ser-Tyr-Gly-Leu-Arg-Pro-Gly-NH2, leuprolide, triptorelin, nafarelin, buserelin, goserelin, cetrorelix, ganirelix, azaline-B, degarelix and abarelix.

Methods for synthesizing LHRH peptides and analogs are well documented and are within the ability of a person of ordinary skill in the art as exemplified in the references listed supra. Further synthetic procedures are provided in the following examples. The following examples also illustrate methods for synthesizing the targeted cytotoxic compounds of the present invention. Specific targeting of therapeutic or cytotoxic agents allows selective destruction of a tumor expressing a receptor specific for a biologically active peptide. For example, a tumor expressing a LHRH receptor includes a neoplasm of the lung, breast, prostate, colon, brain, gastrointestinal tract, neuroendocrine axis, liver, or kidney (see Schaer et al., Int. J. Cancer, 70:530-537, 1997; Chave et al., Br. J. Cancer 82(1):124-130, 2000; Evans et al., Br. J. Cancer 75(6):798-803, 1997).

In some embodiments, the targeting moiety, e.g., LHRH analog, used in the invention is hydrophilic, and is therefore water soluble. In some embodiments, such targeted constructs are used in treatment paradigms in which this feature is useful, e.g., compared to conjugates comprising hydrophobic analogs. Hydrophilic analogs described herein can be soluble in blood, cerebrospinal fluid, and other bodily fluids, as well as in urine, which may facilitate excretion by the kidneys. This feature can be useful, e.g., in the case of a composition that would otherwise exhibit undesirable liver toxicity. The invention also discloses specific hydrophilic elements (e.g., incorporation of a PEG linker, and other examples in the art) for incorporation into peptide analogs, allowing modulation of the analog's hydrophilicity to adjust for the chemical and structural nature of the various conjugated cytotoxic agents.

In some embodiments, the targeting moiety is an antibody mimetic such as a monobody, e.g., an ADNECTIN™ (Bristol-Myers Squibb, New York, N.Y.), an Affibody® (Affibody AB, Stockholm, Sweden), Affilin, nanofitin (affitin, such as those described in WO 2012/085861, an Anticalin™, an avimers (avidity multimers), a DARPin™, a Fynomer™, Centyrin™, a Humabody®, or a Kunitz domain peptide. In certain cases, such mimetics are artificial peptides or proteins with a molar mass of about 3 to 20 kDa. Nucleic acids and small molecules may be antibody mimetic.

In another example, a targeting moiety can be an aptamer, which is generally an oligonucleotide (e.g., DNA, RNA, or an analog or derivative thereof) that binds to a particular target, such as a polypeptide. In some embodiments, the targeting moiety is a polypeptide (e.g., an antibody that can specifically bind a tumor marker). In certain embodiments, the targeting moiety is an antibody or a fragment thereof. In certain embodiments, the targeting moiety is an Fc fragment of an antibody.

In another example, a targeting moiety may be a non-immunoreactive ligand. For example, the non-immunoreactive ligand may be insulin, insulin-like growth factors I and II, lectins, apoprotein from low density lipoprotein, etc. as disclosed in US 20140031535 to Jeffrey, the contents of which are incorporated herein by reference in their entirety. Any protein or peptide comprising a lectin disclosed in WO2013181454 to Radin, the contents of which are incorporated herein by reference in their entirety, may be used as a targeting moiety.

In another example, the conjugate of the invention may target a hepatocyte intracellularly and a hepatic ligand may be used as a targeting moiety. Any hepatic ligand disclosed in US 20030119724 to Ts'o et al., the contents of which are incorporated herein by reference in their entirety, such as the ligands in FIG. 1, may be used. The hepatic ligand specifically binds to a hepatic receptor, thereby directing the conjugate into cells having the hepatic receptor.

In another example, a targeting moiety may interact with a protein that is overexpressed in tumor cells compared to normal cells. The targeting moiety may bind to a chaperonin protein, such as Hsp90, as disclosed in US 20140079636 to Chimmanamada et al., the contents of which are incorporated herein by reference in their entirety. The targeting moiety may be an Hsp90 inhibitor, such as geldanamycins, macbecins, tripterins, tanespimycins, and radicicols.

In another example, the targeted construct may have a terminal half-life of longer than about 72 hours and a targeting moiety may be selected from Table 1 or 2 of US 20130165389 to Schellenberger et al., the contents of which are incorporated herein by reference in their entirety. The targeting moiety may be an antibody targeting delta-like protein 3 (DLL3) in disease tissues such as lung cancer, pancreatic cancer, skin cancer, etc., as disclosed in WO2014125273 to Hudson, the contents of which are incorporated herein by reference in their entirety. The targeting moiety may also any targeting moiety in WO2007137170 to Smith, the contents of which are incorporated herein by reference in their entirety. The targeting moiety binds to glypican-3 (GPC-3) and directs the conjugate to cells expressing GPC-3, such as hepatocellular carcinoma cells.

In some embodiments, a target of the targeting moiety may be a marker that is exclusively or primarily associated with a target cell, or one or more tissue types, with one or more cell types, with one or more diseases, and/or with one or more developmental stages. In some embodiments, a target can comprise a protein (e.g., a cell surface receptor, transmembrane protein, glycoprotein, etc.), a carbohydrate (e.g., a glycan moiety, glycocalyx, etc.), a lipid (e.g., steroid, phospholipid, etc.), and/or a nucleic acid (e.g., a DNA, RNA, etc.).

In another embodiment, targeting moieties may be peptides for regulating cellular activity. For example, the targeting moiety may bind to Toll Like Receptor (TLR). It may be a peptide derived from vaccinia virus A52R protein such as a peptide comprising SEQ ID No. 13 as disclosed in U.S. Pat. No. 7,557,086, a peptide comprising SEQ ID No. 7 as disclosed in U.S. Pat. No. 8,071,553 to Hefeneider, et al., or any TLR binding peptide disclosed in WO 2010141845 to McCoy, et al., the contents of each of which are incorporated herein by reference in their entirety. The A52R derived synthetic peptide may significantly inhibit cytokine production in response to both bacterial and viral pathogen associated molecular patterns, and may have application in the treatment of inflammatory conditions that result from ongoing toll-like receptor activation.

In another embodiment, targeting moieties many be amino acid sequences or single domain antibody fragments for the treatment of cancers and/or tumors. For example, targeting moieties may be an amino acid sequence that binds to Epidermal Growth Factor Receptor 2 (HER2). Targeting moieties may be any HER2-binding amino acid sequence described in US 20110059090, U.S. Pat. Nos. 8,217,140, and 8,975,382 to Revets, et al., the contents of each of which are incorporated herein by reference in their entirety. The targeting moiety may be a domain antibody, a single domain antibody, a VHH, a humanized VHH or a camelized VH.

In another embodiment, targeting moieties may be peptidomimetic macrocycles for the treatment of disease. For example, targeting moieties may be peptidomimetic macrocycles that bind to the growth hormone-releasing hormone (GHRH) receptor, such as a peptidomimetic macrocycle comprising an amino acid sequence which is at least about 60% identical to GHRH 1-29 and at least two macrocycle-forming linkers as described in US20130123169 to Kawahata et al., the contents of which are incorporated herein by reference in their entirety. In another embodiment, the peptidomimetic macrocycle targeting moiety may be prepared by introducing a cross-linker between two amino acid residues of a polypeptide as described in US 20120149648 and US 20130072439 to Nash et al., the contents of each of which are incorporated herein by reference in their entirety. Nash et al. teaches that the peptidomimetic macrocycle may comprise a peptide sequence that is derived from the BCL-2 family of proteins such as a BH3 domain. The peptidomimetic macrocycle may comprise a BID, BAD, BIM, BIK, NOXA, PUMA peptides.

In another embodiment, targeting moieties may be polypeptide analogues for transport to cells. For example, the polypeptide may be an Angiopep-2 polypeptide analog. It may comprising a polypeptide comprising an amino acid sequence at least 80% identical to SEQ ID No.97 as described in US 20120122798 to Castaigne et al., the contents of which are incorporated herein by reference in their entirety. Additionally, polypeptides may transport to cells, such as liver, lung, kidney, spleen, and muscle, such as Angiopep-4b, Angiopep-5, Angiopep-6, and Angiopep-7 polypeptide as described in EP 2789628 to Beliveau et al., the contents of each of which are incorporated herein by reference in their entirety.

In another embodiment, targeting moieties may be homing peptides to target liver cells in vivo. For example, the melittin delivery peptides that are administered with RNAi polynucleotides as described in U.S. Pat. No. 8,501,930 Rozema, et al., the contents of which are incorporated herein by reference in their entirety, may be used as targeting moieties. In addition, delivery polymers provide membrane penetration function for movement of the RNAi polynucleotides from the outside the cell to inside the cell as described in U.S. Pat. No. 8,313,772 to Rozema et al., the contents of each of which are incorporated herein by reference in their entirety. Any delivery peptide disclosed by Rozema et al. may be used as targeting moeities.

In another embodiment, targeting moieties may be structured polypeptides to target and bind proteins. For example, polypeptides with sarcosine polymer linkers that increase the solubility of structured polypeptides, as described in WO 2013050617 to Tite, et al., the contents of which are incorporated herein by reference in their entirety, may be used as targeting moieties. Additionally, polypeptide with variable binding activity produced by the methods described in WO 2014140342 to Stace, et al., the contents of which are incorporated herein by reference in their entirety. The polypeptides may be evaluated for the desired binding activity.

In another embodiment, modifications of the targeting moieties affect a compound's ability to distribute into tissues. For example, a structure activity relationship analysis was completed on a low orally bioavailable cyclic peptide and the permeability and clearance was determined as described in Rand, A C., et al., Medchemcomm. 2012, 3(10): 1282-1289, the contents of which are incorporated herein by reference in their entirety. Any of the cyclic peptide disclosed by Rand et al., such as N-methylated cyclic hexapeptides, may be used as targeting moieties.

In another embodiment, targeting moieties may be a polypeptide which is capable of internalization into a cell. For example, targeting moieties may be an Alphabody capable of internalization into a cell and specifically binding to an intracellular target molecule as described in US 20140363434 to Lasters, et al., the contents of which are incorporated herein by reference in their entirety. As taught by Lasters et al., an ‘Alphabody’ or an ‘Alphabody structure’ is a self-folded, single-chain, triple-stranded, predominantly alpha-helical, coiled coil amino acid sequence, polypeptide or protein. The Alphabody may be a parallel Alphabody or an anti-parallel Alphabody. Moreover, targeting moieties may be any Alphabody in the single-chain Alphabody library used for the screening for and/or selection of one or more Alphabodies that specifically bind to a target molecule of interest as described in WO 2012092970 to Desmet et al., the contents of which are incorporated herein by reference in their entirety.

In another embodiment, targeting moieties may consist of an affinity-matured heavy chain-only antibody. For example, targeting moieties may be any V_{H} heavy chain-only antibodies produced in a transgenic non-human mammal as described in US 20090307787 to Grosveld et al., the contents of which are incorporated herein by reference in their entirety.

In another embodiment, targeting moieties may bind to the hepatocyte growth factor receptor “HGFr” or “cMet”. For example, targeting moieties may be a polypeptide moiety that is conjugated to a detectable label for diagnostic detection of cMet as described in U.S. Pat. No. 9,000,124 to Dransfield et al., the contents of which are incorporated herein by reference in their entirety. Additionally, targeting moieties may bind to human plasma kallikrein and may comprise BPTI-homologous Kunitz domains, especially LACI homologues, to bind to one or more plasma (and/or tissue) kallikreins as described in WO 1995021601 to Markland et al., the contents of which are incorporated herein by reference in their entirety.

In another embodiment, targeting moieties are evolved from weak binders and anchor-scaffold conjugates having improved target binding and other desired pharmaceutical properties through control of both synthetic input and selection criteria. Any target binding element identified in US 20090163371 to Stern et al., the contents of which are incorporated herein by reference in their entirety, may be used as a targeting moiety. Moreover, targeting moieties may be macrocyclic compounds that bind to inhibitors of apoptosis as described in WO 2014074665 to Borzilleri et al., the contents of which are incorporated herein by reference in their entirety.

In another embodiment, targeting moieties may comprise pre-peptides that encode a chimeric or mutant lantibiotic. For example, targeting moieties may be pre-tide that encode a chimera that was accurately and efficiently converted to the mature lantibiotic, as demonstrated by a variety of physical and biological activity assays as described in U.S. Pat. No. 5,861,275 to Hansen, the contents of which are incorporated herein by reference in their entirety. The mixture did contain an active minor component with a biological activity.

In another embodiment, targeting moieties may comprise a leader peptide of a recombinant manganese superoxide dismutase (rMnSOD-Lp). For example, rMnSOD-Lp which delivers cisplatin directly into tumor cells as described in Borrelli, A., et al., Chem Biol Drug Des. 2012, 80(1):9-16, the contents of which are incorporated herein by reference in their entirety, may be used a targeting moiety.

In another embodiment, the targeting moiety may be an antibody for the treatment of glioma. For example, an antibody or antigen binding fragment which specifically binds to JAMM-B or JAM-C as described in U.S. Pat. No. 8,007,797 to Dietrich et al., the contents of which are incorporated herein by reference in their entirety, may be used as a targeting moiety. JAMs are a family of proteins belonging to a class of adhesion molecules generally localized at sites of cell-cell contacts in tight junctions, the specialized cellular structures that keep cell polarity and serve as barriers to prevent the diffusion of molecules across intercellular spaces and along the basolateral-apical regions of the plasma membrane.

In another embodiment, the targeting moiety may be a target interacting modulator. For example, nucleic acid molecules capable of interacting with proteins associated with the Human Hepatitis C virus or corresponding peptides or mimetics capable of interfering with the interaction of the native protein with the HIV accessory protein as described in WO 2011015379 and U.S. Pat. No. 8,685,652, the contents of each of which are incorporated herein by reference in their entirety, may be used as a targeting moiety.

In another embodiment, the targeting moiety may bind with biomolecules. For example, any cystine-knot family small molecule polycyclic molecular scaffolds were designed as peptidomimetics of FSH and used as peptide-vaccine as described in U.S. Pat. No. 7,863,239 to Timmerman, the contents the contents of which are incorporated herein by reference in their entirety, may be used as targeting moieties.

In another embodiment, the targeting moiety may bind to integrin and thereby block or inhibit integrin binding. For example, any highly selective disulfide-rich dimer molecules which inhibit binding of α4β7 to the mucosal addressin cell adhesion molecule (MAdCAM) as described in WO 2014059213 to Bhandari, the contents of which are incorporated herein by reference in their entirety, may be used as a targeting moiety. Any inhibitor of specific integrins-ligand interactions may be used as a targeting moiety. The conjugates comprising such target moieties may be effective as anti-inflammatory agents for the treatment of various autoirnmune diseases.

In another embodiment, the targeting moiety may comprise novel peptides. For example, any cyclic peptide or mimetic that is a serine protease inhibitor as described in WO 2013172954 to Wang et al., the contents of which are incorporated herein by reference in their entirety, may be used as a targeting moiety. Additionally, targeting moieties may comprise a targeting peptide that is used in the reduction of cell proliferation and the treatment of cancer. For example, a peptide composition inhibiting the trpv6 calcium channel as described in US 20120316119 to Stewart, the contents of which are incorporated herein by reference in their entirety, may be used as a targeting moiety.

In another embodiment, the targeting moiety may comprise a cyclic peptide. For example, any cyclic peptides exhibit various types of action in vivo, as described in US20100168380 and WO 2008117833 to Suga et al., and WO 2012074129 to Higuchi et al., the contents of each of which are incorporated herein by reference, may be used as targeting moieties. Such cyclic peptide targeting moieties have a stabilized secondary structure and may inhibit biological molecule interactions, increase cell membrane permeability and the peptide's half-life in blood serum.

In another embodiment, the targeting moiety may consist of a therapeutic peptide. For example, peptide targeting moieties may be an AP-1 signaling inhibitor, such as a peptide analog comprising SEQ ID No. 104 of U.S. Pat. No. 8,946,381B2 to Fear that is used for the treatment of wounds, a peptide comprising SEQ ID No. 108 in U.S. Pat. No. 8,822,409B2 to Milech, et al. that is used to treat acute respiratory distress syndrome (ARDS), or a neuroprotective AP-1 signaling inhibitory peptide that is a fusion peptide comprising a protein transduction domain having the amino acid sequence of SEQ ID NO: 1 and a peptide having the sequence of SEQ ID NO:54 as described in U.S. Pat. No. 8,063,012 to Watt, the contents of each of which are incorporated herein by reference in their entirety. In another example, the targeting moiety may be any biological modulator isolated from biodiverse gene fragment libraries as described in U.S. Pat. No. 7,803,765 and EP1754052 to Watt, any inhibitor of c-Jun dimerization as described in EP1601766 and EP1793841 to Watt, any peptide inhibitors of CD40L signaling as described in U.S. Pat. No. 8,802,634 and US20130266605 to Watt, or any peptide modulators of cellular phenotype as described in US20110218118 to Watt, the contents of each of which are incorporated herein by reference in their entirety.

In another embodiment, the targeting moiety may consist of a characterized peptide. For example, any member of the screening libraries created from bioinformatic source data to theoretically predict the secondary structure of a peptide as described in EP1987178 to Watt et al., any peptide identified from peptide libraries that are screened for antagonism or inhibition of other biological interactions by a reverse hybrid screening method as described by EP1268842 to Hopkins, et al., the contents of each of which are incorporated herein by reference in their entirety, may be used as a targeting moiety. Additionally, targeting moieties may be cell-penetrating peptides. For example, any cell-penetrating peptides linked to a cargo that are capable of passing through the blood brain barrier as described by US20140141452A1 to Watt, et al., the contents of which are incorporated herein by reference, may be used a targeting moiety.

In another embodiment, the targeting moiety may comprise a LHRH antagonist, agonist, or analog. For example, the targeting moiety may be Cetrorelix, a decapeptide with a terminal acid amide group (AC-D-Nal(2)-D-pCl-Phe-D-Pal(3)-Ser-Tyr-D-Cit-Leu-Arg-Pro-D-Ala-NH2) as described in U.S. Pat. Nos. 4,800,191, 6,716,817, 6,828,415, 6,867,191, 7,605,121, 7,718,599, 7,696,149 (Zentaris Ag), or pharmaceutically active decapeptides such as SB-030, SB-075 (cetrorelix) and SB-088 disclosed in EP 0 299 402 (Asta Pharma), the contents of each of which are incorporated herein by reference in their entirety. In another example, the targeting moiety may be LHRH analogues such as D-/L-MeI (4-[bis(2-chloroethyl)amino]-D/L-phenylalanine), cyclopropanealkanoyl, aziridine-2-carbonyl, epoxyalkyl, 1,4-naphthoquinone-5-oxycarbonyl-ethyl, doxorubicinyl (Doxorubicin, DOX), mitomicinyl (Mitomycin C), esperamycinyl or methotrexoyl, as disclosed in U.S. Pat. No. 6,214,969 to Janaky et al., the contents of which are incorporated herein by reference in their entirety.

In another embodiment, the targeting moiety may be any cell-binding molecule disclosed in U.S. Pat. Nos. 7,741,277 or 7,741,277 to Guenther et al. (Aeterna Zentaris), the contents of which are incorporated herein by reference in their entirety, such as octamer peptide, nonamer peptide, decamer peptide, luteinizing hormone releasing hormone (LHRH), [D-Lys6]-LHRH, LHRH analogue, LHRH agonist, Triptorelin ([D-Trp6]-LHRH), LHRH antagonist, bombesin, bombesin analogue, bombesin antagonist, somatostatin, somatostatin analogue, serum albumin, human serum albumin (HSA). These cell-binding molecules may be conjugated with disorazoles.

In another embodiment, targeting moieties may bind to growth hormone secretagogue (GHS) receptors, including ghrelin analogue ligands of GHS receptors. For example, targeting moieties may be any triazole derivatives with improved receptor activity and bioavailability properties as ghrelin analogue ligands of growth hormone secretagogue receptors as describe by U.S. Pat. No. 8,546,435 to Aicher, at al. (Aeterna Zentaris), the contents of which are incorporated herein by reference in their entirety.

In some embodiments, the targeting moiety X is an aptide or bipodal peptide. X may be any D-Aptamer-Like Peptide (D-Aptide) or retro-inverso Aptide which specifically binds to a target comprising: (a) a structure stabilizing region comprising parallel, antiparallel or parallel and antiparallel D-amino acid strands with interstrand noncovalent bonds; and (b) a target binding region I and a target binding region II comprising randomly selected n and m D-amino acids, respectively, and coupled to both ends of the structure stabilizing region, as disclosed in US Pat. Application No. 20140296479 to Jon et al., the contents of which are incorporated herein by reference in their entirety. X may be any bipodal peptide binder (BPB) comprising a structure stabilizing region of parallel or antiparallel amino acid strands or a combination of these strands to induce interstrand non-covalent bonds, and target binding regions I and II, each binding to each of both termini of the structure stabilizing region, as disclosed in US Pat. Application No. 20120321697 to Jon et al., the contents of which are incorporated herein by reference in their entirety. X may be an intracellular targeting bipodal-peptide binder specifically binding to an intracellular target molecule, comprising: (a) a structure-stabilizing region comprising a parallel amino acid strand, an antiparallel amino acid strand or parallel and antiparallel amino acid strands to induce interstrand non-covalent bonds; (b) target binding regions I and II each binding to each of both termini of the structure-stabilizing region, wherein the number of amino acid residues of the target binding region I is n and the number of amino acid residues of the target binding region II is m; and (c) a cell-penetrating peptide (CPP) linked to the structure-stabilizing region, the target binding region I or the target binding region II, as disclosed in US Pat. Application No. 20120309934 to Jon et al., the contents of which are incorporated herein by reference in their entirety. X may be any bipodal peptide binder comprising a β-hairpin motif or a leucine-zipper motif as a structure stabilizing region comprising two parallel amino acid strands or two antiparallel amino acid strands, and a target binding region I linked to one terminus of the first of the strands of the structure stabilizing region, and a target binding region II linked to the terminus of the second of the strands of the structure stabilizing region, as disclosed in US Pat. Application No. 20110152500 to Jon et al., the contents of which are incorporated herein by reference in their entirety. X may be any bipodal peptide binder targeting KPI as disclosed in WO2014017743 to Jon et al, any bipodal peptide binder targeting cytokine as disclosed in WO2011132939 to Jon et al., any bipodal peptide binder targeting transcription factor as disclosed in WO201132941 to Jon et al., any bipodal peptide binder targeting G protein-coupled receptor as disclosed in WO2011132938 to Jon et al., any bipodal peptide binder targeting receptor tyrosine kinase as disclosed in WO2011132940 to Jon et al., the contents of each of which are incorporated herein by reference in their entireties. X may also be bipodal peptide binders targeting cluster differentiation (CD7) or an ion channel.

In another embodiment, the targeting moiety may be a bicyclic peptide or a modified bicyclic peptide, as disclosed in WO2015063465, EP2464727, WO2013050617, WO2016067035, EP233518, US20140249292, US20140256596, EP2970954, U.S. Pat. No. 9,518,081, EP2393520, US20160046928, US20160031939, or US20160046673 (Bicycle Therapeutics), the contents of which are incorporated heren by reference in their entirety.

In some embodiments, the target, target cell or marker is a molecule that is present exclusively or predominantly on the surface of malignant cells, e.g., a tumor antigen. In some embodiments, a marker is a prostate cancer marker. In some embodiments the target can be an intra-cellular protein.

In some embodiments, a marker is a breast cancer marker, a colon cancer marker, a rectal cancer marker, a lung cancer marker, a pancreatic cancer marker, a ovarian cancer marker, a bone cancer marker, a renal cancer marker, a liver cancer marker, a neurological cancer marker, a gastric cancer marker, a testicular cancer marker, a head and neck cancer marker, an esophageal cancer marker, or a cervical cancer marker.

The targeting moiety directs the conjugates to specific tissues, cells, or locations in a cell. The target can direct the conjugate in culture or in a whole organism, or both. In each case, the targeting moiety binds to a receptor that is present on the surface of or within the targeted cell(s), wherein the targeting moiety binds to the receptor with an effective specificity, affinity and avidity. In other embodiments the targeting moiety targets the conjugate to a specific tissue such as the liver, kidney, lung or pancreas. The targeting moiety can target the conjugate to a target cell such as a cancer cell, such as a receptor expressed on a cell such as a cancer cell, a matrix tissue, or a protein associated with cancer such as tumor antigen. Alternatively, cells comprising the tumor vasculature may be targeted. Targeting moieties can direct the conjugate to specific types of cells such as specific targeting to hepatocytes in the liver as opposed to Kupffer cells. In other cases, targeting moieties can direct the conjugate to cells of the reticular endothelial or lymphatic system, or to professional phagocytic cells such as macrophages or eosinophils.

In some embodiments the target is member of a class of proteins such as receptor tyrosine kinases (RTK) including the following RTK classes: RTK class I (EGF receptor family) (ErbB family), RTK class II (Insulin receptor family), RTK class III (PDGF receptor family), RTK class IV (FGF receptor family), RTK class V (VEGF receptors family), RTK class VI (HGF receptor family), RTK class VII (Trk receptor family), RTK class VIII (Eph receptor family), RTK class IX (AXL receptor family), RTK class X (LTK receptor family), RTK class XI (TIE receptor family), RTK class XII (ROR receptor family), RTK class XIII (DDR receptor family), RTK class XIV (RET receptor family), RTK class XV (KLG receptor family), RTK class XVI (RYK receptor family) and RTK class XVII (MuSK receptor family).

In some embodiments the target is a serine or threonine kinase, G-protein coupled receptor, methyl CpG binding protein, cell surface glycoprotein, cancer stem cell antigen or marker, carbonic anhydrase, cytolytic T lymphocyte antigen, DNA methyltransferase, an ectoenzyme, a glycosylphosphatidylinositol-anchored co-receptor, a glypican-related integral membrane proteoglycan, a heat shock protein, a hypoxia induced protein, a multi drug resistant transporter, a Tumor-associated macrophage marker, a tumor associated carbohydrate antigen, a TNF receptor family member, a transmembrane protein, a tumor necrosis factor receptor superfamily member, a tumour differentiation antigen, a zinc dependent metallo-exopeptidase, a zinc transporter, a sodium-dependent transmembrane transport protein, a member of the SIGLEC family of lectins, or a matrix metalloproteinase.

Other cell surface markers are useful as potential targets for tumor-homing therapeutics, including, for example HER-2, HER-3, EGFR, the folate receptor and neurotensin receptors (NTSR1 or NTSR2).

Neurotensin is a neuropeptide involved in dopamine signaling and thermoregulation. Neurotensin receptor 1 (NTSR1), a G protein coupled receptor (GPCR), is normally expressed only in the brain and colon, but some cancers can overexpress NTSR1. For example, NTSR1 is expressed in majority of pancreatic cancers, and has high expression in subsets of non-small cell lung cancer (NSCLC) and ductal breast carcinomas. NTSR1 is involved in the growth of expressing cancer cells, and NTSR1 expression correlates with poor prognosis. Therefore, NTSR1 is considered a therapeutic target for anticancer drug development.

In some embodiments, the targeting moiety binds to NTSR1. The targeting moiety may be a peptide or a small molecule. Non-limiting examples include neurontensin (a 13 amino acid peptide shown below) and its derivatives/analogs/fragments.

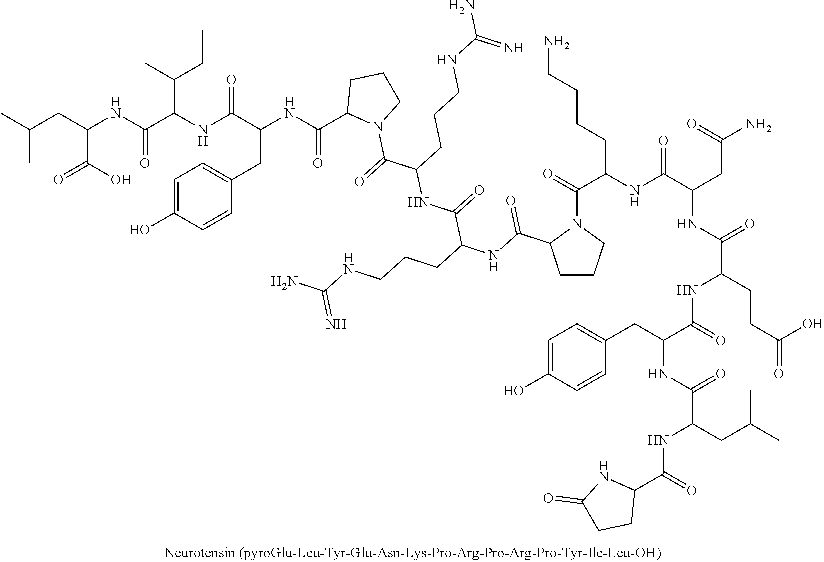 [SMILES: CCC(C)C(NC(=O)C(Cc1ccc(O)cc1)NC(=O)C1CCCN1C(=O)C(CCCNC(=N)N)NC(=O)C(CCCNC(=N)N)NC(=O)C1CCCN1C(=O)C(CCCCN)NC(=O)C(CC(N)=O)NC(=O)C(CCC(=O)O)NC(=O)C(Cc1ccc(O)cc1)NC(=O)C(CC(C)C)NC(=O)C1CCC(=O)N1)C(=O)NC(CC(C)C)C(=O)O]

The natural ligand (neurotensin) and its analogs have very high affinity for the receptor, internalize rapidly, and degrade within the cell after internalization. The six C-terminal amino acids are the targeting domain for NTSR1.

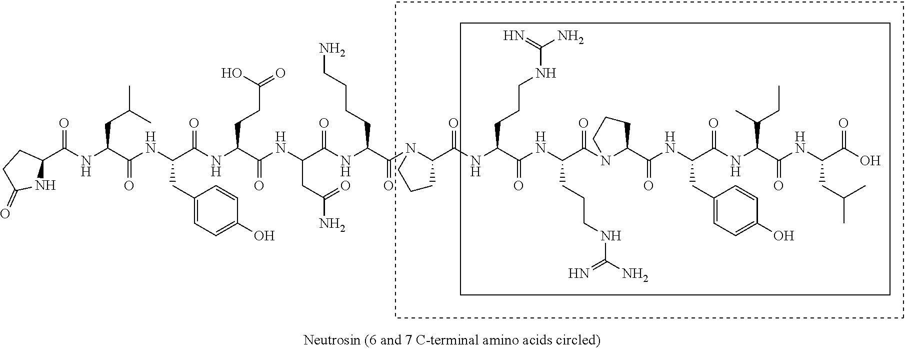 [SMILES: CCC(C)[C@H](NC(=O)[C@H](Cc1ccc(O)cc1)NC(=O)[C@@H]1CCCN1C(=O)[C@H](CCCNC(=N)N)NC(=O)[C@H](CCCNC(=N)N)NC(=O)[C@@H]1CCCN1C(=O)[C@H](CCCCN)NC(=O)C(CC(N)=O)NC(=O)[C@H](CCC(=O)O)NC(=O)[C@H](Cc1ccc(O)cc1)NC(=O)[C@H](CC(C)C)NC(=O)[C@@H]1CCC(=O)N1)C(=O)N[C@@H](CC(C)C)C(=O)O]

In some embodiments, the targeting moiety that binds to NTSR1 comprises the targeting domain of neurotensin or derivative thereof, e.g., six or seven C-terminal amino acids of neurotensin. The targeting moiety may further comprise a linking amino acid which attaches to the targeting domain of neurotensin to a variety of releasable linkers. The targeting domain of neurotensin may be modified to increase stability. For example, an isoleucine group on isoleucine residue may be replaced with tert-leucine for greater stability. A targeting moiety comprise seven C-terminal amino acids of neurotensin with tert-leucine modification is shown below:

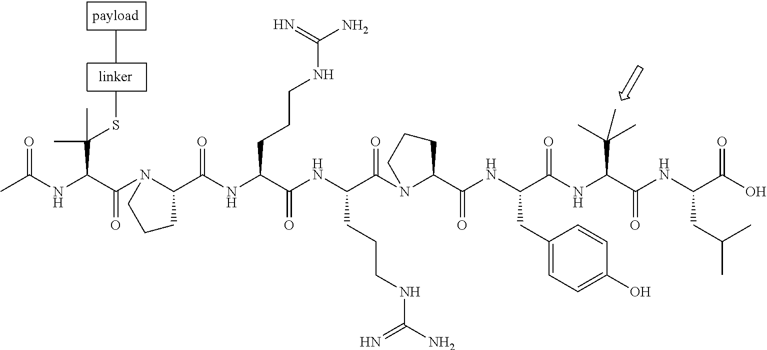

### An Example of NTSR1-Binding Conjugates

In some embodiments, the targeting moiety may be neurotensin, neurotensin (6-13), or any neurotensin derivative/analog. The neurotensin derivate/analog may have stronger binding to NTSR1 than neurotensin. As a non-limiting example, the neurotensin analog may be NMeArg-Arg-Pro-Tyr-Tle-Leu-OH or DArg-Arg-Pro-Tyr-Ile-TMSAla-OH. Neurotensin and its derivatives/analogs cause internalization of NTSR1.

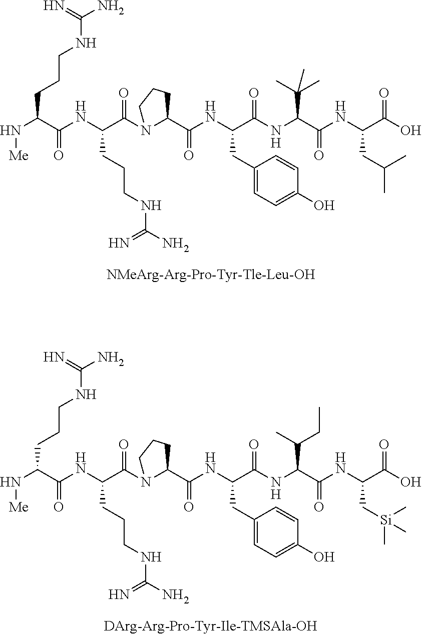 [SMILES: CN[C@@H](CCCNC(=N)N)C(=O)N[C@@H](CCCNC(=N)N)C(=O)N1CCC[C@H]1C(=O)N[C@@H](Cc1ccc(O)cc1)C(=O)N[C@H](C(=O)N[C@@H](CC(C)C)C(=O)O)C(C)(C)C ; CCC(C)[C@H](NC(=O)[C@H](Cc1ccc(O)cc1)NC(=O)[C@@H]1CCCN1C(=O)[C@H](CCCNC(=N)N)NC(=O)[C@@H](CCCNC(=N)N)NC)C(=O)N[C@@H](C[Si](C)(C)C)C(=O)O]

In some embodiments, the targeting moiety that binds to NTSR1 may be a biased agonist of NTSR1 such as ML314 (2-cyclopropyl-6,7-dimethoxy-4-(4-(2-methoxyphenyl)-piperazin-1-yl)quinazoline, structure shown below; compound 32 in Pinkerton et al., ACS Medicinal Chemistry Letters, vol. 4:846 (2013), the contents of which are incorporated herein by reference in their entirety); any small molecule neurotensin receptor agonist disclosed in WO 2014/100501 to Pinkerton et al., the contents of which are incorporated herein by reference in their entirety, such as compounds of Formula I-XIV or in claims 49-54; any small molecule neurotensin receptor agonist disclosed in WO 2015/200534 to Pinkerton et al, the contents of which are incorporated herein by reference in their entirety, such as compounds of Formula I-VIII, in paragraph [00196], or in claims 3, 12 and 30; or any derivative thereof.

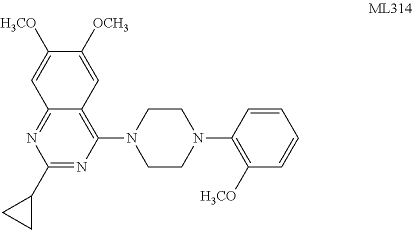 [SMILES: COc1cc2nc(C3CC3)nc(N3CCN(c4ccccc4OC)CC3)c2cc1OC]

In other embodiments, the targeting moiety binds a target such as CD19, CD70, CD56, PSMA, alpha integrin, CD22, CD138, EphA2, AGS-5, Nectin-4, HER2, GPMNB, CD74 and Le.

In some embodiments the target is a protein listed in Table A.

TABLE A
Non-limiting examples of proteins that may be targeted

| | | | |
|---|---|---|---|
| 5T4 | CD64 | GPIIb/IIIa receptors | PDGFRbeta |
| A20/TNFAIP3 | CD68 | GPR161/RE2 | P-glycoprotein |
| ABCB5 | CD70 | Guanylyl cyclase receptor C | Podoplanin |
| ABCG2 | CD80 | HA-CD44v3 | PON1 |
| AFP | CD86 | HER2/ERBB2 | PRAME |
| ALCAM/CD166 | CD90 | HIF1alpha | PSAM |
| ALDH1A1 | CD96 | HIF-2 | PTEN |
| Apelin J Receptor | CEACAM-5/cd66e | HLA-DR | RAAG12 |
| APN/CD13 | CEACAM-6 | Hsp90 | RON |
| AXL | c-KIT | IGE receptor | sialyl-Le(x) |
| B7H4 | c-Maf | IGF-1R | sialyl-Le(x) |
| BCMA | c-Met | IL-1 alpha | sialyl-Tn |
| BCRP/ABCG2 | Cripto/TDGF-1 | IL-11R | Sigma |
| | | | Receptor/Pgrmc1 |
| BMI-1 | CSFR | IL-1R | SLC34A2 |
| CA9 | CXCR1 | IL-23R | SLC44A4 |
| CAIX | CXCR1 | IL-2R | SLITRK6 |
| mmp | CXCR4 | IL-3 R | SOX2 |
| CanAg | disialylgalactosylgloboside | IL-4R | STAT-3 |
| CD117 | DLL4 | IL-6 R | STEAP-1 |
| CD11a | DNMT1 | Indegrin alpha 6 | STRO-1 |
| CD11b | DNMT3A | iNOS | Tenasin-C |
| CD136 | DNMT3B | Insulin receptor | TF antigen |
| CD138 | DNMT3L | L1CAM | TIM-3 |
| CD14 | EDB (Fibronectin extra | LGR5 | Tissue Factor |
| | domain B) | | (CD142) |
| CD15 | EGFR VIII | LIV-1 (SLC39A6), Zip6 | Tn antigen |
| CD152 (CTLA-4) | E-NPP3/CD203c | LRP | TNFR |
| CD172A | Epcam/TROP1 | MAGE-A3 | TRAIL-R1 |
| CD19 | EphA1 | MBD1 | TRAIL-R2 |
| CD20 | EphA2 | MBD2 | Transferrin receptor |
| CD204 | ERBB3 | MBD4 | TRK-A |
| CD206 | FAP | Mesothelin | TRK-B |
| CD22 | FGFR1 | Metadherin/MTDH/AEG-1 | Trop-2/EGP-1 |
| CD24 | FGFR2 | MICL | UHRF1 |
| CD25 | FGFR3 | MMP-2 | UHRF2 |
| CD26 | FGFR4 | MMP-9 | VEGFR1 |
| CD27 (CD70L) | Fibronectin | MRP1 | VEGFR2 |
| CD28 | Folate receptor | Muc-1 | VEGFR3 |
| CD3 | FRb | MUC16/CA-125 | ZBTB33 |
| CD30 | Galbg4 | Mushai-1 | ZBTB4 |
| CD33 | GD2 ganglioside | NaPi2b | EphA3 |
| CD34 | GD3 ganglioside | Nectin-4 | EphA4 |
| CD38 | GLI-1 | Nestin | EphA5 |
| CD40 | GLI-2 | Neurotensin receptor 1 | EphA6 |
| CD41 | globo-H | NF2 | EphA7 |
| CD44 | GLUT1 | Notch1 | EphA8 |
| CD45 | Glycoprotein NMB | Notch2 | EphB1 |
| CD45.1 | glycosphingolipid P₁ | Notch3 | EphB2 |
| CD45.2 | GM2 ganglioside | Notch4 | EphB3 |
| CD47/IAP | GP130 | Ovastacin | EphB4 |
| CD52 | GPC3 Glypican-3 | PDGFRalpha | EphB5 |
| EphB6 | GRP78 | | |

In certain embodiments, the targeting moiety or moieties of the conjugate are present at a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of targeting moieties of the conjugate may also be expressed in terms of proportion to the active agent(s), for example, in a ratio of ligand to active agent of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

C. Internal Linker Moieties

The conjugates contain one or more internal linker moieties attaching the active agents and targeting moieties. The internal linker moiety, Y, is bound to one or more active agents and one or more targeting moieties to form a conjugate. The internal linker moiety Y is attached to the targeting moiety X and the active agent Z by functional groups independently selected from an ester bond, disulfide, thioether, amide, acylhydrazone, ether, carbamate, carbonate, carbon-carbon bond, and urea. Alternatively the internal linker moiety can be attached to either the targeting moiety or the active drug by a non-cleavable group such as provided by the conjugation between a thiol and a maleimide, or between an azide and an alkyne. The internal linker is independently selected from the group consisting alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

In some embodiments, the internal linker moiety comprises a cleavable functionality designed to be cleaved in an intracellular manner. The cleavable functionality may be hydrolyzed in vivo or may be designed to be hydrolyzed enzymatically, for example by Cathepsin B, or may be a pH-sensitive linker. A “cleavable” linker, as used herein, refers to any linker which can be cleaved physically or chemically. Examples for physical cleavage may be cleavage by light, radioactive emission or heat, while examples for chemical cleavage include cleavage by re-dox-reactions, hydrolysis, pH-dependent cleavage or cleavage by enzymes.

In some embodiments the alkyl chain of the internal linker moiety may optionally be interrupted by one or more atoms or groups selected from —O—, —C(═O)—, —NR, —O—C(═O)—NR—, —S—, —S—S—. The internal linker moiety may be selected from dicarboxylate derivatives of succinic acid, glutaric acid or diglycolic acid.

In some embodiments, the internal linker Y moiety may be X′-R¹-Y′-R²-Z′ and the conjugate can be a compound according to Formula Ia:

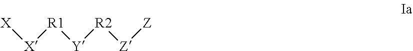

wherein X is a targeting moiety defined above; Z is an active agent; X′, R¹, Y′, R² and Z′ are as defined herein.

X′ is either absent or independently selected from carbonyl, amide, urea, amino, ester, aryl, arylcarbonyl, aryloxy, arylamino, one or more natural or unnatural amino acids, thio or succinimido; R¹ and R² are either absent or comprised of alkyl, substituted alkyl, aryl, substituted aryl, polyethylene glycol (2-30 units); Y′ is absent, substituted or unsubstituted 1,2-diaminoethane, polyethylene glycol (2-30 units) or an amide; Z′ is either absent or independently selected from carbonyl, amide, urea, amino, ester, aryl, arylcarbonyl, aryloxy, arylamino, thio or succinimido. In some embodiments, the internal linker can allow one active agent molecule to be linked to two or more ligands, or one ligand to be linked to two or more active agent molecule.

In some embodiments, the internal linker Y moiety may be Aₘ and the conjugate can be a compound according to Formula Ib:

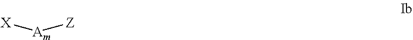

wherein A is defined herein, m=0-20.

A in Formula Ia is a spacer unit, either absent or independently selected from the following substituents. For each substituent, the dashed lines represent substitution sites with X, Z or another independently selected unit of A wherein the X, Z, or A can be attached on either side of the substituent:

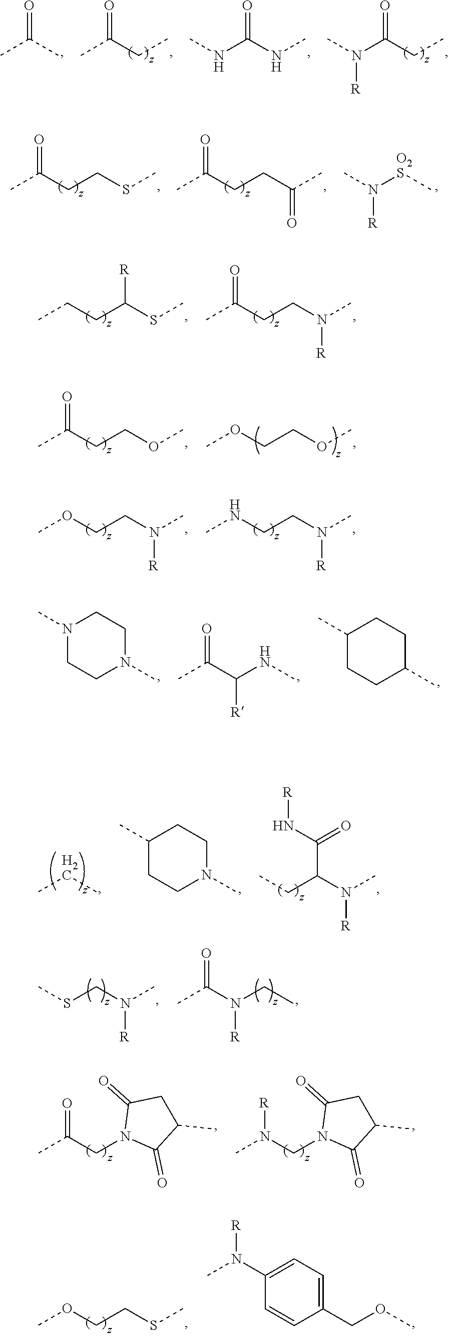

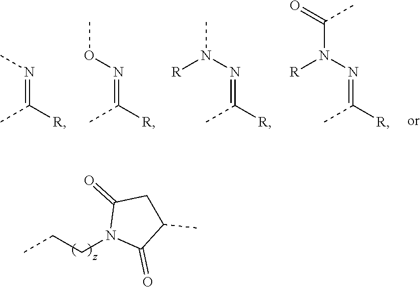

wherein z=0-40, R is H or an optionally substituted alkyl group, and R′ is any side chain found in either natural or unnatural amino acids.

In some embodiments, the conjugate may be a compound according to Formula Ic:

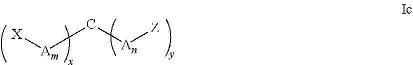

wherein A is defined above, m=0-40, n=0-40, x=1-5, y=1-5, and C is a branching element defined herein.

C in Formula Ic is a branched unit containing three to six functionalities for covalently attaching spacer units, ligands, or active drugs, selected from amines, carboxylic acids, thiols, or succinimides, including amino acids such as lysine, 2,3-diaminopropanoic acid, 2,4-diaminobutyric acid, glutamic acid, aspartic acid, and cysteine.

In some embodiments, the internal linker moiety may be cleavable and is cleaved to release the active agent. In one embodiment, the internal linker may be cleaved by an enzyme. As a non-limiting example, the internal linker moiety may be a polypeptide moiety, e.g. AA in WO2010093395 to Govindan, the contents of which are incorporated herein by reference in their entirety, that is cleavable by intracellular peptidase. Govindan teaches AA in the linker moiety may be a di, tri, or tetrapeptide such as Ala-Leu, Leu-Ala-Leu, and Ala-Leu-Ala-Leu. In another example, the cleavable internal linker moiety may be a branched peptide. The branched peptide linker may comprise two or more amino acid moieties that provide an enzyme cleavage site. Any branched peptide linker disclosed in WO1998019705 to Dubowchik, the contents of which are incorporated herein by reference in their entirety, may be used as an internal linker moiety in the conjugate of the present invention. As another example, the linker may comprise a lysosomally cleavable polypeptide disclosed in U.S. Pat. No. 8,877,901 to Govindan et al., the contents of which are incorporated herein by reference in their entirety. As another example, the internal linker may comprise a protein peptide sequence which is selectively enzymatically cleavable by tumor associated proteases, such as any Y and Z structures disclosed in U.S. Pat. No. 6,214,345 to Firestone et al., the contents of which are incorporated herein by reference in their entirety.

In one embodiment, the cleaving of the internal linker moiety is non-enzymatic. Any linker disclosed in US 20110053848 to Cleemann et al., the contents of which are incorporated herein by reference in their entirety, may be used. For example, the linker may be a non-biologically active linker represented by formula (I).

In one embodiment, the internal linker moiety may be a beta-glucuronide linker disclosed in US 20140031535 to Jeffrey, the contents of which are incorporated herein by reference in their entirety. In another embodiment, the internal linker may be a self-stabilizing linker such as a succinimide ring, a maleimide ring, a hydrolyzed succinimide ring or a hydrolyzed maleimide ring, disclosed in US20130309256 to Lyon et al., the contents of which are incorporated herein by reference in their entirety. In another embodiment, the internal linker may be a human serum albumin (HAS) linker disclosed in US 20120003221 to McDonagh et al., the contents of which are incorporated herein by reference in their entirety. In another embodiment, the internal linker may comprise a fullerene, e.g., C₆₀, as disclosed in US 20040241173 to Wilson et al., the contents of which are incorporated herein by reference in their entirety. In another embodiment, the internal linker may be a recombinant albumin fused with polycysteine peptide as disclosed in U.S. Pat. No. 8,541,378 to Ahn et al., the contents of which are incorporated herein by reference in their entirety. In another embodiment, the internal linker comprises a heterocycle ring. For example, the internal linker may be any heterocyclic 1,3-substituted five- or six-member ring, such as thiazolidine, disclosed in US 20130309257 to Giulio, the contents of which are incorporated herein by reference in their entirety.

In some embodiments, the internal linker moiety Y may be a Linker Unit (LU) as described in US2011/0070248, the contents of which are incorporated herein by reference in their entirety. In formula (I) where the Ligand Drug Conjugate has formula L-(LU-D)ₚ the targeting moiety X corresponds to L (the Ligand unit) and the active agent Z corresponds to D (the drug unit).

The conjugate X-Y-Z can be a conjugate as described in WO2014/134486, the contents of which are incorporated herein by reference in their entirety. The targeting moiety X, corresponds to the cell binding agent, CBA in formula (I′) or (I) as reproduced here, wherein the internal linker moiety Y and the active agent Z together correspond to the remainder of the formula (in parentheses).

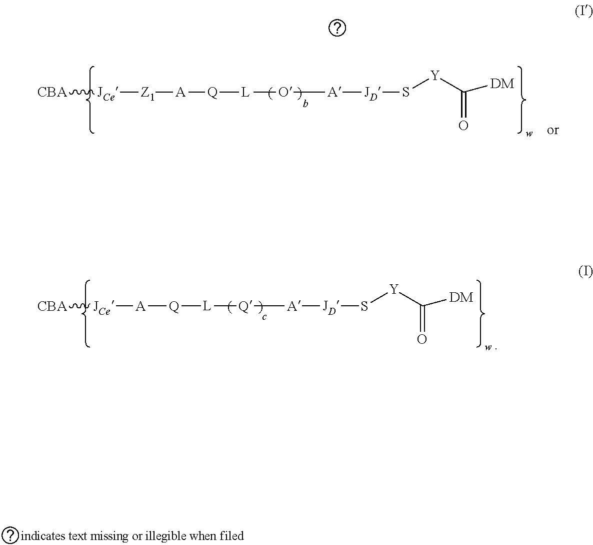

The conjugate X-Y-Z can be a conjugate as described in U.S. Pat. No. 7,601,332, the contents of which are incorporated herein by reference in their entirety, wherein conjugates are described as follows, and the targeting moiety X corresponds to V (the vitamin receptor binding moiety), the active agent Z corresponds to D (drugs and includes analogs or derivatives thereof), and the internal linker moiety Y corresponds to the bivalent linker (L) which can comprise one or more components selected from spacer linkers (ls), releasable linkers (lr), and heteroatom linkers (lH), and combinations thereof, in any order:
V-L-D
V-(lᵣ)_{c}-D
V-(lₛ)ₐ-D
V-(lₛ)ₐ-(lᵣ)_{c}-D
V-(lᵣ)_{c}-(lₛ)ₐ-D
V-(l_{H})_{b}-(l_{I})_{c}-D
V-(lᵣ)_{c}-(l_{H})_{b}-D
V-(l_{H})_{d}-(lᵣ)_{c}-(l_{H})ₑ-D
V-(lₛ)ₐ-(l_{H})_{b}-(lᵣ)_{c}-D
V-(lᵣ)_{c}-(l_{H})_{b}-(lₛ)ₐ-D
V-(l_{H})_{d}-(lₛ)ₐ-(lᵣ)_{c}-(l_{H})ₑ-D
V-(l_{H})_{d}-(lᵣ)_{c}-(lₛ)ₐ-(l_{H})ₑ-D
V-(l_{H})_{d}-(lₛ)ₐ-(l_{H})_{b}-(lᵣ)_{c}-(l_{H})ₑ-D
V-(l_{H})_{d}-(lᵣ)_{c}-(l_{H})_{b}-(lₛ)ₐ-(l_{H})ₑ-D
V-(lₛ)ₐ-(lᵣ)_{c}-(l_{H})_{b}-D
V-[(lₛ)ₐ-(l_{H})_{b}]_{d}-(lᵣ)_{c}-(l_{H})ₑ-D

In some embodiments, the conjugate is a small molecule drug conjugates (SMDC). In some embodiments, the conjugate comprises a targeting moiety that binds to a somatostatin receptor (SSTR) such as SSTR2. In some embodiments, the conjugate comprises a SSTR binding ligand, such as somatostatin, octreotide, octreotate, vapreotide, pasireotide, lanreotide, seglitide, Tyr3-octreotate (TATE), cyclo(AA-Tyr-DTrp-Lys-Thr-Phe), or derivatives thereof, as a targeting moiety. In some embodiments, the conjugate comprises DM1 as an active agent.

In one example, the targeted constructs of the present invention comprise a conjugate of DM1 connected to a SSTR2 ligand with an internal disulfide linker moiety and further comprise a maleimide group as a reacting group, attached via a pH-sensitive β-carbamoyl sulfone external linker, that reacts a functional group on albumin and analogs. Compound 1 is a non-limiting example.

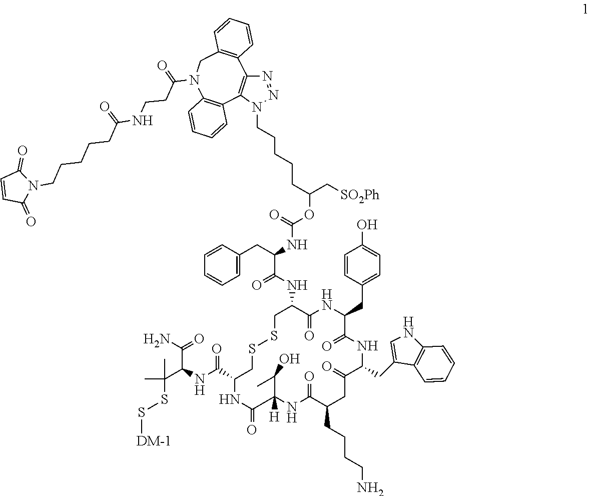

In some embodiments, the targeted constructs of the present invention comprise a conjugate of DM1 connected to a SSTR2 ligand with an internal disulfide linker moiety and further comprise a PEG unit as a pharmacokinetic modulating unit attached via a pH-sensitive β-carbamoyl sulfone external linker. Compound 2 is a non-limiting example.

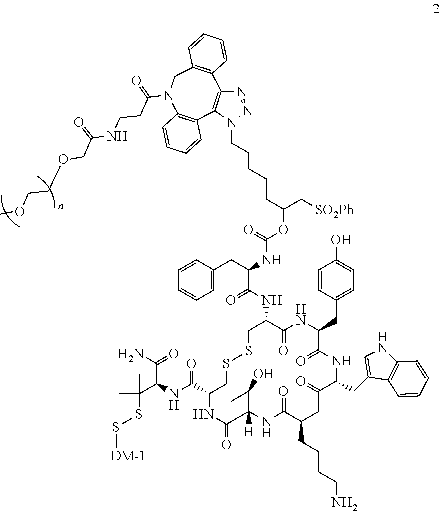

External Linkers

The optional external linker in Embodiments 1), 2) and 3) is independently selected from the group consisting of alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

In some embodiments, the external linker moiety comprises a cleavable functionality designed to be cleaved in an extracellular manner. The external linker may be cleavable in tumor microenvironment, e.g., by a pH-dependent or hypoxia-dependent cleavage that relies upon conditions in the tumor microenvironment or by extracellular proteases such as matrix metalloproteinases.

In some embodiments, the external linker may comprise one or more atoms or groups selected from —O—, —C(═O)—, —NR, —O—C(═O)—NR—, —S—, —S—S—.

Non-limiting Examples of Conjugates

In some embodiments of Embodiment 2), the conjugates comprise a reacting group that binds to albumin. The reacting group may be connected to the internal linker of the conjugate with an acid-labile external linker. The targeting moiety may bind to a neurotensin receptor (NTSR). The internal linker may comprise a cleavable group, such as a disulfide group, a carbamate group, a peptide that is cleavable by a protease, such as a Cathepsin B cleavable sequence. The active agent may be a small molecule.

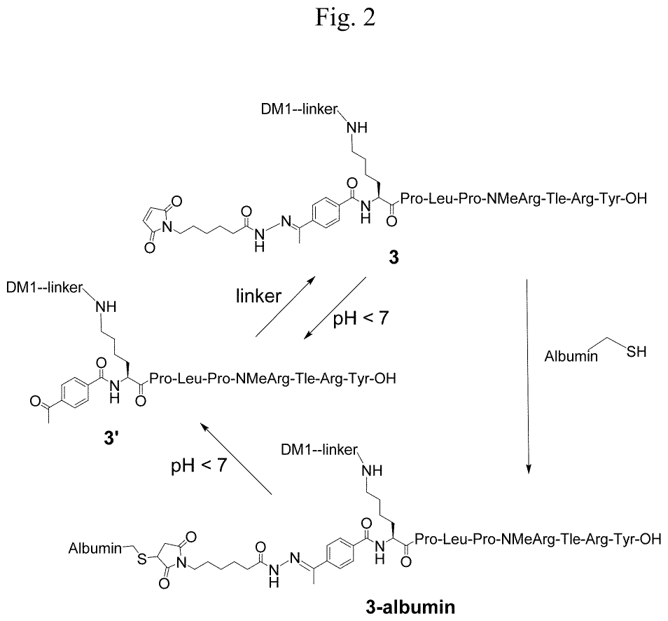

FIG. 2 shows a scheme of producing an albumin-binding conjugate. Compound 3′ reacts with a reagent comprising maleimide to produce Compound 3, which is an albumin-binding conjugate. Compound 3 reacts with albumin in situ to produce Compound 3-albumin. Once Compound 3-albumin is delivered to a tumor site with a pH of less than about 7, Compound 3-albumin is released from albumin and coverts back to Compound 3′. The internal linker is then cleaved to release the active agent.

In some embodiments, the conjugate comprises a targeting moiety that binds to NTSR1. The targeting moiety may be neurotensin (6-13) or a neurotensin analog/derivative. The active agent may be DM1. The active agent may also be pyrrolobenzodiazepine (PBD) or a PDB dimer, or derivatives/analogs thereof. In one example, the conjugate has a structure of:

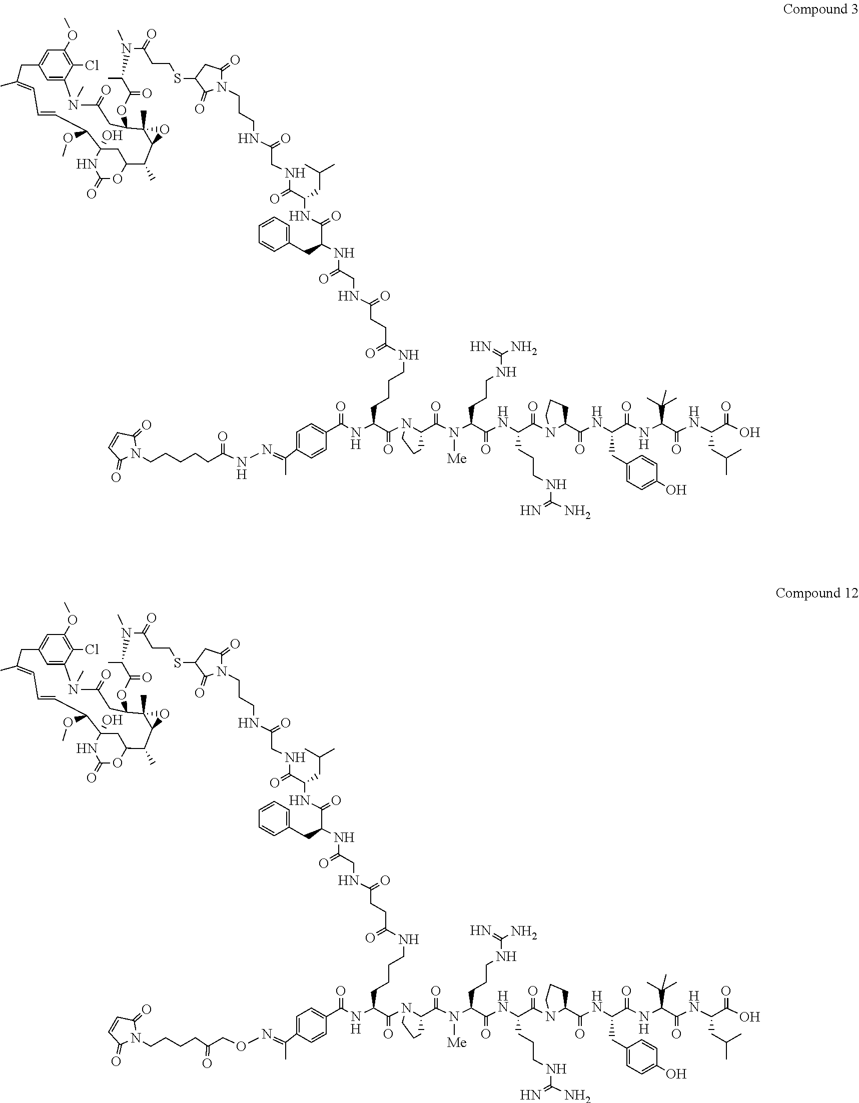

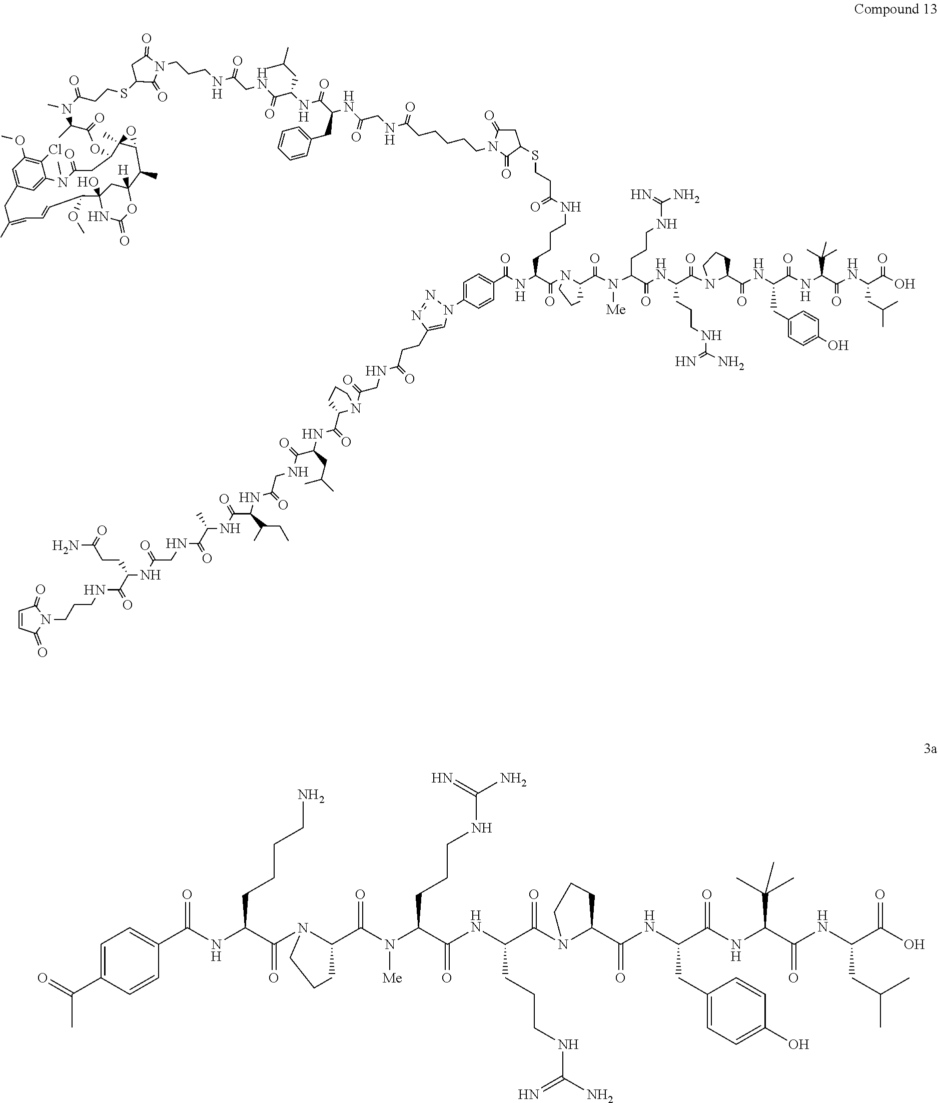

### II. Pharmaceutical Compositions and Formulations

In some embodiments, compositions are administered to humans, human patients, healthy volunteers, or any other subjects. For the purposes of the present disclosure, the phrase “active ingredient” generally refers to the targeted constructs of the present invention to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to animals, e.g. mammals, rodents, or avians. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with one or more excipients and/or one or more other accessory ingredients including solvents and aqueous solutions, and then, if necessary and/or desirable, dissolving, dividing, sterilizing, filling or shaping and/or packaging the product into a desired single- or multi-use units.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.05% and 100%, e.g., between 0.1 and 75%, between 0.5 and 50%, between 1-30%, between 5-80%, at least 80% (w/w) active ingredient.

The targeted constructs of the present invention can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the targeted constructs of the present invention.

Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, retinoid-like excipient (e.g. excipients that resemble vitamin A), coloring agents, coating agents, sweetening, flavoring, and/or perfuming agents can be present in the composition, according to the judgment of the formulator.

In one embodiment, the formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

Administration

The targeted constructs of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier.

The formulations described herein contain an effective amount of the targeted constructs of the present invention in a pharmaceutical carrier appropriate for administration to an individual in need thereof. The may be administered parenterally (e.g., by injection or infusion). The formulations or variations thereof may be administered in any manner including enterally, topically (e.g., to the eye), or via pulmonary administration. In some embodiments the formulations are administered topically.

A. Parenteral Formulations

The targeted constructs of the present invention can be formulated for parenteral delivery, such as injection or infusion, in the form of a solution, suspension or emulsion. The formulation can be administered systemically, regionally or directly to the organ or tissue to be treated.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the particles can be prepared in water or another solvent or dispersing medium suitably mixed with one or more pharmaceutically acceptable excipients including, but not limited to, surfactants, dispersants, emulsifiers, pH modifying agents, and combinations thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or targeted constructs.

The formulation is typically buffered to a pH of 3-8 for parenteral administration upon reconstitution. Suitable buffers include, but are not limited to, phosphate buffers, acetate buffers, and citrate buffers. If using 10% sucrose or 5% dextrose, a buffer may not be required.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the particles in the required amount in the appropriate solvent or dispersion medium with one or more of the excipients listed above, as required, followed by filtered sterilization. Generally, dispersions are prepared by incorporating the various sterilized particles into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the particle plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of targeted constructs . Acceptable solvents include, for example, water, Ringer's solution, phosphate buffered saline (PBS), and isotonic sucrose, dextrose or sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral administration may also contain one or more tonicity agents to adjust the isotonic range of the formulation. Suitable tonicity agents are well known in the art and some examples include glycerin, sucrose, dextrose, mannitol, sorbitol, sodium chloride, and other electrolytes.

Solutions, suspensions, or emulsions for parenteral administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

B. Mucosal Topical Formulations

The targeted constructs of the present invention can be formulated for topical administration to a mucosal surface. Suitable dosage forms for topical administration include creams, ointments, salves, sprays, gels, lotions, emulsions, liquids, and transdermal patches. The formulation may be formulated for transmucosal transepithelial, or transendothelial administration. The compositions contain one or more chemical penetration enhancers, membrane permeability agents, membrane transport agents, emollients, surfactants, stabilizers, and combination thereof. In some embodiments, the targeted constructs can be administered as a liquid formulation, such as a solution or suspension, a semi-solid formulation, such as a lotion or ointment, or a solid formulation. In some embodiments, the targeted constructs are formulated as liquids, including solutions and suspensions, such as eye drops or as a semi-solid formulation, to the mucosa, such as the eye or vaginally or rectally.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the dispersion of one liquid in another and promote the formation of a stable mixture, or emulsion, of oil and water or water in oil. Common emulsifiers are: anaionic, cataionic and nonionic surfactants or micttures of surfactants, certain animal and vegetable oils, and various polar surface active compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

Dosing

The present invention provides methods comprising administering the targeted constructs of the present invention to a subject in need thereof. The targeted constructs of the present invention may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g, two or more administrations of the single unit dose. As used herein, a “single unit dose” is a dose of any therapeutic administed in one dose/at one time/single route/single point of contact, i.e., single administration event. As used herein, a “total daily dose” is an amount given or prescribed in 24 hr period. It may be administered as a single unit dose. In one embodiment, the monomaleimide compounds of the present invention are administed to a subject in split doses. The monomaleimide compounds may be formulated in buffer only or in a formulation described herein.

Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous).

A. Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

B. Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the monomaleimide compounds then depends upon its rate of dissolution which, in turn, may depend upon crystal size and crystalline form. Alternatively, delayed absorption of a parenterally administered monomaleimide compound may be accomplished by dissolving or suspending the monomalimide in an oil vehicle. Injectable depot forms are made by forming microencapsule matrices of the monomaleimide compounds in biodegradable polymers such as polylactide-polyglycolide. Depending upon the ratio of monomaleimide compounds to polymer and the nature of the particular polymer employed, the rate of monomaleimide compound release can be controlled. Examples of other biodegradable polymers include, but are not limited to, poly(orthoesters) and poly(anhydrides). Depot injectable formulations may be prepared by entrapping the monomaleimide compounds in liposomes or microemulsions which are compatible with body tissues.

C. Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 um to 500 um. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

D. Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### III. Methods of Making the Targeted Constructs

The targeted constructs and conjugates can be made by many different synthetic procedures. The conjugates can be prepared from internal linker moieties having one or more reactive coupling groups or from one or more internal linker precursors capable of reacting with a reactive coupling group on an active agent or targeting moiety to form a covalent bond.

The conjugates can be prepared from an internal linker moiety precursor capable of reacting with a reactive coupling group on an active agent or targeting moiety to form the internal linker covalently bonded to the active agent or targeting moiety.

The internal linker moiety precursor can be a diacid or substituted diacid. Diacids, as used herein, can refer to substituted or unsubstituted alkyl, heteroalkyl, aryl, or heteroaryl compounds having two or more carboxylic acid groups, preferably having between 2 and 50, between 2 and 30, between 2 and 12, or between 2 and 8 carbon atoms. Suitable diacids can include oxalic acid, malonic acid, succinic acid, glutaric acid, adipic acid, pimelic acid, suberic acid, azelaic acid, sebacic acid, phthalic acid, iso-phthalic acid, terepthalic acid, and derivatives thereof.

The internal linker moiety precursor can be an activated diacid derivative such as a diacid anhydride, diacid ester, or diacid halide. The diacid anhydride can be a cyclic anhydride obtained from the intramolecular dehydration of a diacid or diacid derivative such as those described above. The diacid anhydride can be malonic anhydride, succinic anhydride, glutaric anhydride, adipic anhydride, pimelic anhydride, phthalic anhydride, diglycolic anhydride, or a derivative thereof; preferably succinic anhydride, diglycolic anhydride, or a derivative thereof. The diacid ester can be an activated ester of any of the diacids described above, including methyl and butyl diesters or bis-(p-nitrophenyl) diesters of oxalic acid, malonic acid, succinic acid, glutaric acid, adipic acid, pimelic acid, suberic acid, azelaic acid, sebacic acid, phthalic acid, iso-phthalic acid, terepthalic acid, and derivatives thereof. The diacid halide can include the corresponding acid fluorides, acid chlorides, acid bromides, or acid iodides of the diacids described above. In preferred embodiments the diacid halide is succinyl chloride or diglycolyl chloride. For example, a therapeutic agent having a reactive (—OH) coupling group and a targeting moiety having a reactive (—NH2) coupling group can be used to prepare a conjugate having a disuccinate linker according to the following general scheme.

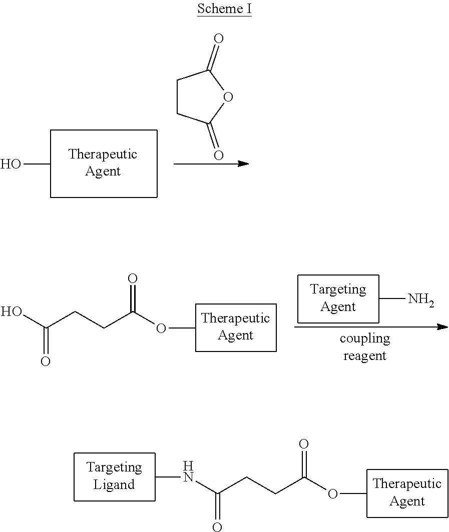

Referring to Scheme I above, the conjugates can be prepared by providing an active agent having a hydroxyl group and reacting it with a succinic anhydride linker precursor to form the conjugate of active agent-succinate-SSPy. A targeting moiety with an available —NH₂ group is reacted with a coupling reagent and the active agent-succinate-SSPy to form the targeting moiety-internal linker-active agent conjugate.

Other functional groups that can be linked to include, but are not limited to, —SH, —COOH, alkenyl, phosphate, sulfate, heterocyclic NH, alkyne and ketone.

The coupling reaction can be carried out under esterification conditions known to those of ordinary skill in the art such as in the presence of activating agents, e.g., carbodiimides (such as diisopropoylcarbodiimide (DIPC)), with or without catalyst such as dimethylaminopyridine (DMAP). This reaction can be carried out in an appropriate solvent, such as dichloromethane, chloroform or ethyl acetate, at a temperature or between about 0° C. and the reflux temperature of the solvent (e.g., ambient temperature). The coupling reaction is generally performed in a solvent such as pyridine or in a chlorinated solvent in the presence of a catalyst such as DMAP or pyridine at a temperature between about 0° C. and the reflux temperature of the solvent (e.g., ambient temperature). In preferred embodiments, the coupling reagent is selected from the group consisting of 4-(2-pyridyldithio)-butanoic acid, and a carbodiimide coupling reagent such as DCC in a chlorinated, ethereal or amidic solvent (such as N,N-dimethylformamide) in the presence of a catalyst such as DMAP at a temperature between about 0° C. and the reflux temperature of the solvent (e.g., ambient temperature).

The conjugates can be prepared by coupling an active agent and/or targeting moiety having one or more reactive coupling groups to an internal linker moiety having complimentary reactive groups capable of reacting with the reactive coupling groups on the active agent or targeting moiety to form a covalent bond. For example, an active agent or targeting moiety having a primary amine group can be coupled to an internal linker having an isothiocyonate group or another amine-reactive coupling group. In some embodiments the internal linker contains a first reactive coupling group capable of reacting with a complimentary functional group on the active agent and a second reactive coupling group different from the first and capable of reacting with a complimentary group on the targeting moiety. In some embodiments one or both of the reactive coupling groups on the internal linker can be protected with a suitable protecting group during part of the synthesis.

In some embodiments, the conjugates may be synthesized with ‘click chemistry’ of the copper ion-catalyzed acetylene-azide cycloaddition reaction. For example, WO2010093395 to Govindan, the contents of which are incorporated herein by reference in their entirety, teaches that the targeting moiety comprises L2, wherein L2 comprises a targeting moiety-coupling end and one or more acetylene or azide groups at the other end. The active agent moiety comprises L1, wherein L1 comprises a defined PEG with azide or acetylene at one end, complementary to the acetylene or azide moiety in L2, and a reactive group such as carboxylic acid or hydroxyl group at the other end. ‘Click chemistry’ between L2 and L1 yields a conjugate comprising the targeting moiety and the active agent.

In some embodiments, the conjugates may be synthesized with thiol-ene ‘click chemistry’. For example, US 20130323169 to Xu et al., the contents of which are incorporated herein by reference in their entirety, teaches preparing a drug conjugate by reacting a sulfhydryl or thiol group (—SH) on the targeting moiety with a double bond on the internal linker moiety.

The conjugates can then be modified to include at least one reacting group that reacts with a functional group on a protein or an engineered protein or polymer or derivatives/analogs/mimics thereof, or be modified to connect to the reacting group or at least one pharmacokinetic modulating unit by an optional external linker with any suitable synthesis route. Alternatively, at least two conjugates can be assembled with external linkers or with ionic bonds or non-covalent bonds to form targeted constructs of the present invention.

### IV. Methods of Using the Targeted Constructs

The targeted constructs of the present invention or formulations containing the targeted constructs of the present invention can be administered to treat any hyperproliferative disease, metabolic disease, infectious disease, inflammatory disease, cancer, or any other disease, as appropriate. The formulations can be used for immunization. The formulations may be delivered to various body parts, such as but not limited to, brain and central nervous system, eyes, ears, lungs, bone, heart, kidney, liver, spleen, breast, ovary, colon, pancreas, muscles, gastrointestinal tract, mouth, skin, to treat diseases associated with such body parts. Formulations may be administered by injection, orally, or topically, typically to a mucosal surface (lung, nasal, oral, buccal, sublingual, vaginally, rectally) or to the eye (intraocularly or transocularly).

In some embodiments, the targeted constructs of the present invention may be combined with at least one other active agent to form a composition. The at least one active agent may be a therapeutic, prophylactic, diagnostic, or nutritional agent. It may be a small molecule, protein, peptide, lipid, glycolipid, glycoprotein, lipoprotein, carbohydrate, sugar, or nucleic acid. The targeted constructs of the present invention and the at least one other active agent may have the same target and/or treat the same disease.

The targeted constructs of the present invention and the at least one other active agent may be administered simultaneously or sequentially. They may be present as a mixture for simultaneous administration, or may each be present in separate containers for sequential administration.

The term “simultaneous administration,” as used herein, is not specifically restricted and means that the targeted constructs and the at least one other active agent are substantially administered at the same time, e.g. as a mixture or in immediate subsequent sequence.

The term “sequential administration,” as used herein, is not specifically restricted and means that the targeted constructs and the at least one other active agent are not administered at the same time but one after the other, or in groups, with a specific time interval between administrations. The time interval may be the same or different between the respective administrations of the targeted constructs and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

In some embodiments, more than one targeted construct of the present invention may be combined to form a composition. The targeted constructs may comprise different conjugates, wherein the conjugates may have different active agents, different linkers, and/or different targeting moieties. The targeted constructs may have different compositions, different active agent loadings, and/or different molecular weights. The targeted constructs in the composition may be administered simultaneously or sequentially. They may be present as a mixture for simultaneous administration, or may each be present in separate containers for sequential administration. Pharmacokinetic properties of the composition, such as Cmax, may be modulated by adjusting the weight percent ratio of the targeted constructs in the composition.

In various embodiments, methods for treating a subject having a cancer are provided, wherein the method comprises administering a therapeutically-effective amount of the targeted constructs of the present invention to a subject having a cancer, suspected of having cancer, or having a predisposition to a cancer. According to the present invention, cancer embraces any disease or malady characterized by uncontrolled cell proliferation, e.g., hyperproliferation. Cancers may be characterized by tumors, e.g., solid tumors or any neoplasm.

In some embodiments, provided is a method for treating a subjection having inflammation, comprising administering a therapeutically-effective amount of the targeted constructs of the present invention to the subject.

In some embodiments, the targeted constructs of the present invention have been found to inhibit cancer and/or tumor growth. They may also reduce, including cell proliferation, invasiveness, and/or metastasis, thereby rendering them useful for the treatment of a cancer.

In some embodiments, the targeted constructs of the present invention may be used to prevent the growth of a tumor or cancer, and/or to prevent the metastasis of a tumor or cancer. In some embodiments, compositions of the present teachings may be used to shrink or destroy a cancer.

In some embodiments, the targeted constructs of the present invention are useful for inhibiting proliferation of a cancer cell including but not limited to mammalian cancer cells. In some instances, the mammalian cancer cells are human cancer cells. In some embodiments, the targeted constructs of the present invention are useful for inhibiting cellular proliferation, e.g., inhibiting the rate of cellular proliferation, preventing cellular proliferation, and/or inducing cell death. In general, the targeted constructs of the present invention can inhibit cellular proliferation of a cancer cell or both inhibiting proliferation and/or inducing cell death of a cancer cell.

The cancers treatable by methods of the present teachings generally occur in mammals. Mammals include, for example, humans, non-human primates, dogs, cats, rats, mice, rabbits, ferrets, guinea pigs horses, pigs, sheep, goats, and cattle. In various embodiments, the cancer is lung cancer, breast cancer, e.g., mutant BRCA1 and/or mutant BRCA2 breast cancer, non-BRCA-associated breast cancer, colorectal cancer, neuroendodrine cancer, ovarian cancer, pancreatic cancer, colorectal cancer, bladder cancer, prostate cancer, cervical cancer, renal cancer, leukemia, central nervous system cancers, myeloma, and melanoma. In some embodiments, the cancer is lung cancer. In certain embodiments, the cancer is human lung carcinoma, ovarian cancer, pancreatic cancer or colorectal cancer.

The targeted constructs of the present invention or formulations containing the targeted constructs of the present invention can be used for the selective tissue delivery of a therapeutic, prophylactic, or diagnostic agent to an individual or patient in need thereof. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. Dosage unit form as used herein refers to physically discrete units suited as unitary dosages for the mammalian subjects to be treated; each unit containing a predetermined quantity of active compound calculated to produce the desired therapeutic.

In various embodiments, conjugates contained within the targeted constructs of the present invention are released in a controlled manner. The release can be in vitro or in vivo.

With respect to conjugates being released in vivo, for example, the conjugates contained within the targeted constructs of the present invention administered to a subject may be protected from a subject's body, and the body may also be isolated from the conjugate until the conjugates are released from the targeted constructs of the present invention.

Thus, in some embodiments, less than about 90%, less than about 80%, less than about 70%, less than about 60%, less than about 50%, less than about 40%, less than about 30%, less than about 20%, less than about 15%, less than about 10%, less than about 5%, or less than about 1% of the total conjugates are released from the targeted constructs of the present invention prior to the targeted constructs being delivered into the body, for example, a treatment site, of a subject. In some embodiments, the conjugate may be released over an extended period of time or by bursts (e.g., amounts of the conjugate are released in a short period of time, followed by a periods of time where substantially no conjugate is released). For example, the conjugates can be released over 6 hours, 12 hours, 24 hours, or 48 hours. In certain embodiments, the conjugates are released over one week or one month.

In some embodiments, the targeted constructs of the present invention may be administered to tumors with a high level of enhanced permeability and retention (EPR) effect. In some embodiments, tumors with a high level of enhanced permeability and retention effect may be identified with imaging techniques. As a non-limited example, iron oxide nanoparticle magnetic resonance imaging may be administered to a patient and EPR effects are measured.

In some embodiments, the targeted constructs of the present invention may be administered to a subject selected with the method disclosed in WO2015017506, the contents of which are incorporated herein by reference in their entirety, the method comprising:

a) administering a contrast agent to the subject;

(b) measuring the level of accumulation of the contrast agent at at least one intended site of treatment; and

(c) selecting the subject based on the level of the accumulation of the contrast agent;

wherein the intended site of treatment is a tumor.

### V. Kits and Devices

The invention provides a variety of kits and devices for conveniently and/or effectively carrying out methods of the present invention. Typically kits will comprise sufficient amounts and/or numbers of components to allow a user to perform multiple treatments of a subj ect(s) and/or to perform multiple experiments.

In one embodiment, the present invention provides kits for inhibiting tumor cell growth in vitro or in vivo, comprising the targeted constructs of the present invention or a combination of the targeted constructs of the present invention, optionally in combination with any other active agents.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the targeted constructs of the present invention in the buffer solution over a period of time and/or under a variety of conditions.

The present invention provides for devices which may incorporate the targeted constructs of the present invention. These devices contain in a stable formulation available to be immediately delivered to a subject in need thereof, such as a human patient. In some embodiments, the subject has cancer.

Non-limiting examples of the devices include a pump, a catheter, a needle, a transdermal patch, a pressurized olfactory delivery device, iontophoresis devices, multi-layered microfluidic devices. The devices may be employed to deliver targeted constructs of the present invention according to single, multi- or split-dosing regiments. The devices may be employed to deliver the targeted constructs of the present invention across biological tissue, intradermal, subcutaneously, or intramuscularly.

It will be appreciated that the following examples are intended to illustrate but not to limit the present invention. Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

### VI. Definitions

The term “compound,” as used herein, is meant to include all stereoisomers, geometric isomers, tautomers, and isotopes of the structures depicted. In the present application, compound is used interechangably with conjugate. Therefore, conjugate, as used herein, is also meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The terms “subject” or “patient,” as used herein, refer to any organism to which the targeted constructs may be administered, e.g., for experimental, therapeutic, diagnostic, and/or prophylactic purposes. Typical subjects include animals (e.g., mammals such as mice, rats, rabbits, guinea pigs, cattle, pigs, sheep, horses, dogs, cats, hamsters, lamas, non-human primates, and humans).

The terms “treating” or “preventing,” as used herein, can include preventing a disease, disorder or condition from occurring in an animal that may be predisposed to the disease, disorder and/or condition but has not yet been diagnosed as having the disease, disorder or condition; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

A “target,” as used herein, shall mean a site to which targeted constructs bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). In still other embodiments, a target may refer to a molecular structure to which a targeting moiety or ligand binds, such as a hapten, epitope, receptor, dsDNA fragment, carbohydrate or enzyme. A target may be a type of tissue, e.g., neuronal tissue, intestinal tissue, pancreatic tissue, liver, kidney, prostate, ovary, lung, bone marrow, or breast tissue.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human.

The term “modulation” is art-recognized and refers to up regulation (i.e., activation or stimulation), down regulation (i.e., inhibition or suppression) of a response, or the two in combination or apart.

“Parenteral administration,” as used herein, means administration by any method other than through the digestive tract (enteral) or non-invasive topical routes. For example, parenteral administration may include administration to a patient intravenously, intradermally, intraperitoneally, intrapleurally, intratracheally, intraossiously, intracerebrally, intrathecally, intramuscularly, subcutaneously, subjunctivally, by injection, and by infusion.

“Topical administration,” as used herein, means the non-invasive administration to the skin, orifices, or mucosa. Topical administrations can be administered locally, i.e., they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

“Enteral administration,” as used herein, means administration via absorption through the gastrointestinal tract. Enteral administration can include oral and sublingual administration, gastric administration, or rectal administration.

“Pulmonary administration,” as used herein, means administration into the lungs by inhalation or endotracheal administration. As used herein, the term “inhalation” refers to intake of air to the alveoli. The intake of air can occur through the mouth or nose.

The terms “sufficient” and “effective,” as used interchangeably herein, refer to an amount (e.g., mass, volume, dosage, concentration, and/or time period) needed to achieve one or more desired result(s). A “therapeutically effective amount” is at least the minimum concentration required to effect a measurable improvement or prevention of at least one symptom or a particular condition or disorder, to effect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are known in the art. The therapeutically effective amounts of compounds and compositions described herein, e.g., for treating specific disorders may be determined by techniques that are well within the craft of a skilled artisan, such as a physician.

The terms “bioactive agent” and “active agent,” as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “prodrug” refers to an agent that is converted into a biologically active form in vitro and/or in vivo. Prodrugs can be useful because, in some situations, they may be easier to administer than the parent compound. For example, a prodrug may be bioavailable by oral administration whereas the parent compound is not. The prodrug may also have improved solubility in pharmaceutical compositions compared to the parent drug. A prodrug may be converted into the parent drug by various mechanisms, including enzymatic processes and metabolic hydrolysis. Harper, N. J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnej ad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogs, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2:S15-27; Wang, W. et al. (1999) Prodrug approaches to the improved delivery of peptide drugs. Curr. Pharm. Des., 5(4):265-87.

The term “biocompatible,” as used herein, refers to a material that along with any metabolites or degradation products thereof that are generally non-toxic to the recipient and do not cause any significant adverse effects to the recipient. In general, biocompatible materials are materials that do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks or even longer.

The term “pharmaceutically acceptable,” as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier,” as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “small molecule,” as used herein, generally refers to an organic molecule that is less than 5000 g/mol in molecular weight, less than 2000 g/mol, less than 1500 g/mol, less than 1000 g/mol, less than 800 g/mol, or less than 500 g/mol. Small molecules are non-polymeric and/or non-oligomeric.

The term “hydrophilic,” as used herein, refers to substances that have strongly polar groups that readily interact with water.

The term “hydrophobic,” as used herein, refers to substances that lack an affinity for water; tending to repel and not absorb water as well as not dissolve in or mix with water.

The term “lipophilic,” as used herein, refers to compounds having an affinity for lipids.

The term “amphiphilic,” as used herein, refers to a molecule combining hydrophilic and lipophilic (hydrophobic) properties. “Amphiphilic material” as used herein refers to a material containing a hydrophobic or more hydrophobic oligomer or polymer (e.g., biodegradable oligomer or polymer) and a hydrophilic or more hydrophilic oligomer or polymer.

The term “targeting moiety,” as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected molecule.

The term “reacting group,” as used herein, refers to any chemical functional group capable of reacting with a second functional group to form a covalent bond. The selection of reacting groups is within the ability of the skilled artisan. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group,” as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene and Wuts., Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphεnylyl)-2-propyloxycarbonyl, 2-bromobenzyloxycarbonyl, tert-butyl₇ tert-butyloxycarbonyl, 1-carbobenzoxamido-2,2,2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

The term “activated ester,” as used herein, refers to alkyl esters of carboxylic acids where the alkyl is a good leaving group rendering the carbonyl susceptible to nucleophilic attack by molecules bearing amino groups. Activated esters are therefore susceptible to aminolysis and react with amines to form amides. Activated esters contain a carboxylic acid ester group —CO₂R where R is the leaving group.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. Throughout the application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl,” as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl,” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

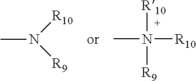

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

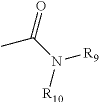

wherein R₉ and R₁₀ are as defined above.

“Aryl,” as used herein, refers to C₅-C₁₀-membered aromatic, heterocyclic, fused aromatic, fused heterocyclic, biaromatic, or bihetereocyclic ring systems. Broadly defined, “aryl,” as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl,” as used herein, refers to an alkyl group substituted with an aryl group (e.g., an aromatic or heteroaromatic group).

The term “carbocycle,” as used herein, refers to an aromatic or non-aromatic ring in which each atom of the ring is carbon.

“Heterocycle” or “heterocyclic,” as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

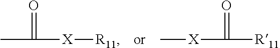

wherein X is a bond or represents an oxygen or a sulfur, and R₁₁ represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analog of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms are boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative sub stituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, or elimination.

In a broad aspect, the permissible substituents include acyclic and cyclic, branched and unbranched, carbocyclic and heterocyclic, aromatic and nonaromatic sub stituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkyl sulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “mean particle size,” as used herein, generally refers to the statistical mean particle size (diameter) of the particles in the composition. The diameter of an essentially spherical particle may be referred to as the physical or hydrodynamic diameter of a spherical particle with an equivalent volume. The diameter of a non-spherical particle may refer to the hydrodynamic diameter. As used herein, the diameter of a non-spherical particle may refer to the largest linear distance between two points on the surface of the particle. Mean particle size can be measured using methods known in the art such as size exclusion chromatography (SEC), dynamic light scattering (DLS), electron microscopy, laser diffraction, MALDI-TOF, zeta potential measurement, AFM, TEM, SEM X-Ray microanalysis, or nanoparticle tracking analysis. Two populations can be said to have a “substantially equivalent mean particle size” when the statistical mean particle size of the first population of particles is within 20% of the statistical mean particle size of the second population of particles; for example, within 15%, or within 10%.

The terms “monodisperse” and “homogeneous size distribution,” as used interchangeably herein, describe a population of particles, microparticles, or nanoparticles all having the same or nearly the same size. As used herein, a monodisperse distribution refers to particle distributions in which 90% of the distribution lies within 5% of the mean particle size.

The term “polydispersity index” is used herein as a measure of the size distribution of an ensemble of particles, e.g., nanoparticles. The polydispersity index can be calculated based on dynamic light scattering measurements.

The terms “polypeptide,” “peptide” and “protein” generally refer to a polymer of amino acid residues. As used herein, the term also applies to amino acid polymers in which one or more amino acids are chemical analogs or modified derivatives of corresponding naturally-occurring amino acids. The term “protein,” as generally used herein, refers to a polymer of amino acids linked to each other by peptide bonds to form a polypeptide for which the chain length is sufficient to produce tertiary and/or quaternary structure. The term “protein” excludes small peptides by definition, the small peptides lacking the requisite higher-order structure necessary to be considered a protein.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays or complementation, e.g., genetic or biochemical. See, for example, Fields et al. (1989) Nature 340:245-246; U.S. Pat. No. 5,585,245 and PCT WO 98/44350.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

The term “pharmaceutically acceptable counter ion” refers to a pharmaceutically acceptable anion or cation. In various embodiments, the pharmaceutically acceptable counter ion is a pharmaceutically acceptable ion. For example, the pharmaceutically acceptable counter ion is selected from citrate, malate, acetate, oxalate, chloride, bromide, iodide, nitrate, sulfate, bisulfate, phosphate, acid phosphate, isonicotinate, acetate, lactate, salicylate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)). In some embodiments, the pharmaceutically acceptable counter ion is selected from chloride, bromide, iodide, nitrate, sulfate, bisulfate, phosphate, acid phosphate, citrate, malate, acetate, oxalate, acetate, and lactate. In particular embodiments, the pharmaceutically acceptable counter ion is selected from chloride, bromide, iodide, nitrate, sulfate, bisulfate, and phosphate.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “bioavailable” is art-recognized and refers to a form of the subject invention that allows for it, or a portion of the amount administered, to be absorbed by, incorporated to, or otherwise physiologically available to a subject or patient to whom it is administered.

### EXAMPLES

### Example 1

### Preparation of an Azide-Functionalized Compound A

Compound A comprises DM-1 as an active agent and a SSTR2 ligand as a targeting moiety. It is synthesized with the following route:

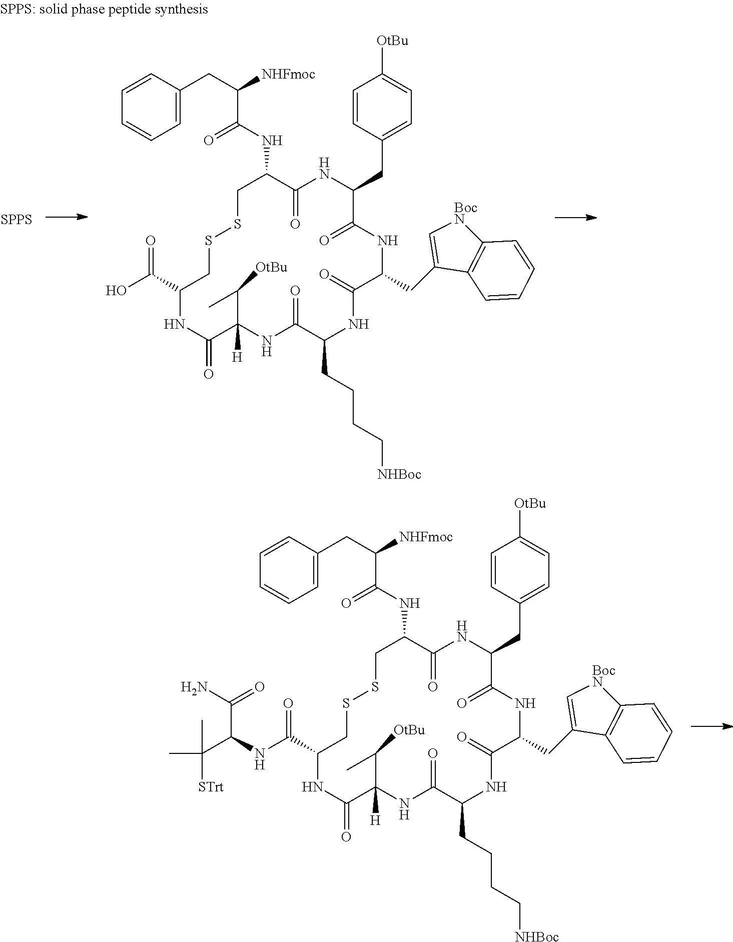

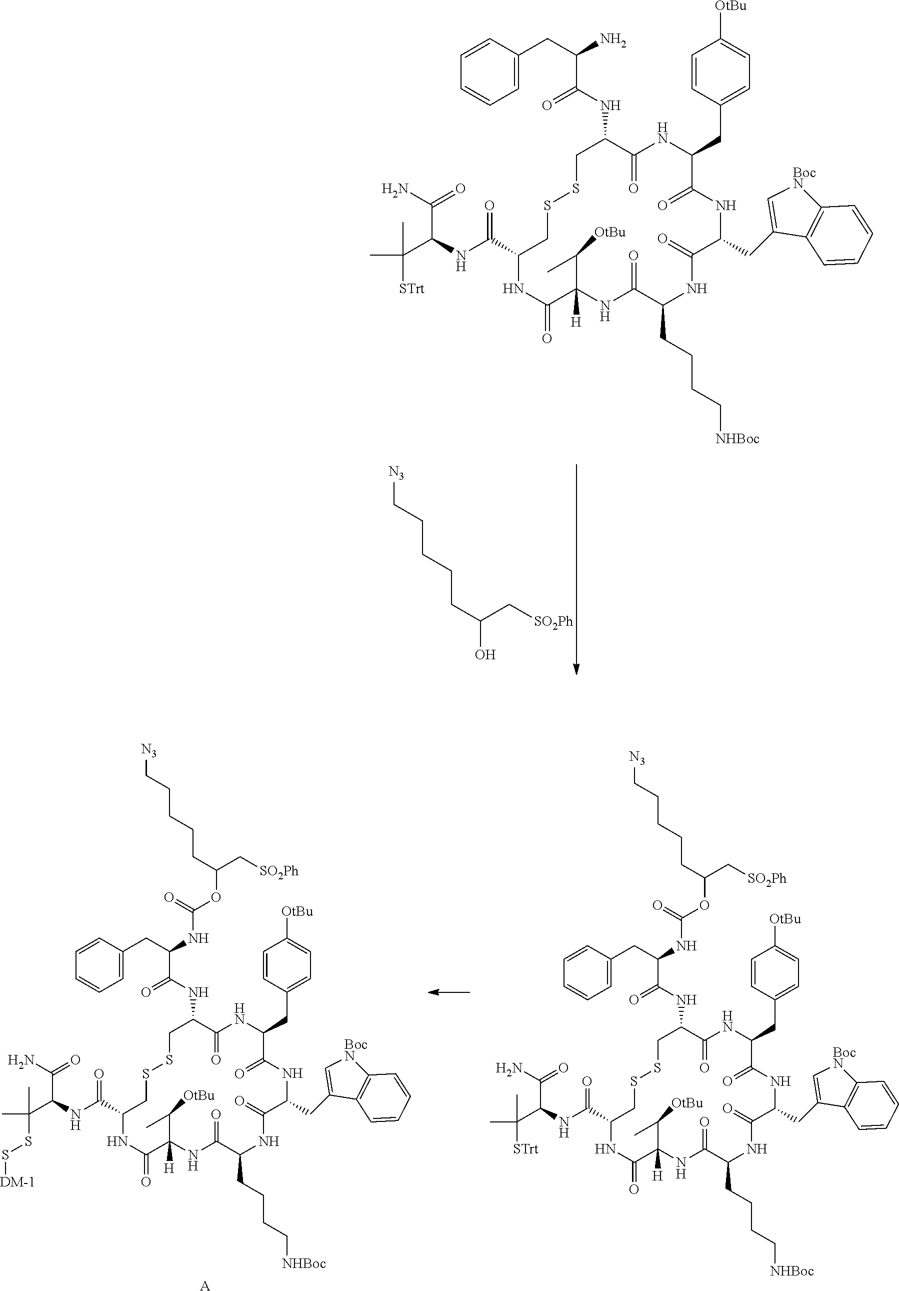

### Example 2

### Preparation of an Albumin-Binding Compound 1

Compound 1 comprises a maleimide group that binds to albumin and can be synthesized from Compound A with the following route:

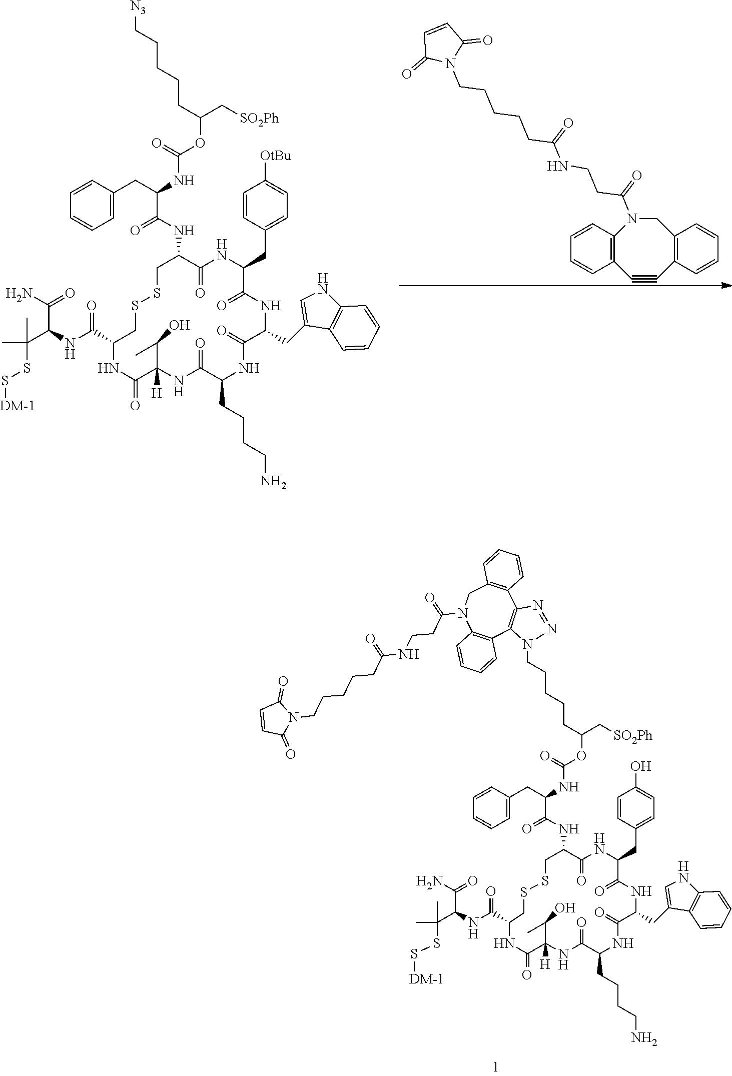

### Example 3

### Preparation of a PEGylated Compound 2

Compound 2 (n=100 to 3000) comprises a PEG unit and can be synthesized from Compound A with the following route:

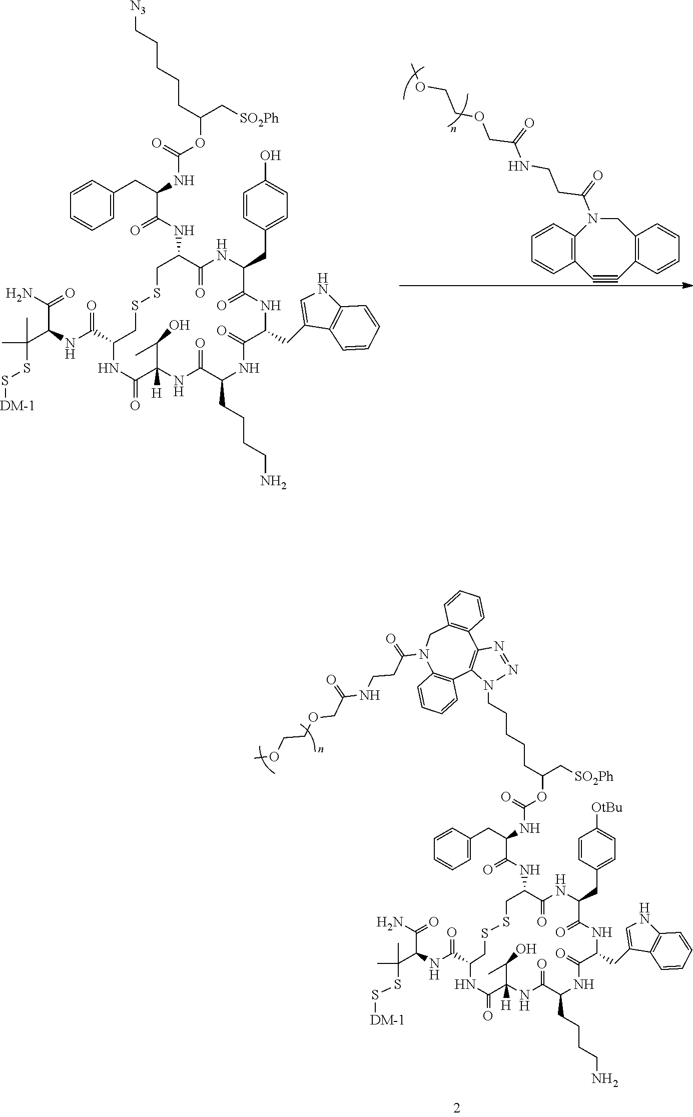

### Example 4

### Preparation of Compounds 3-8

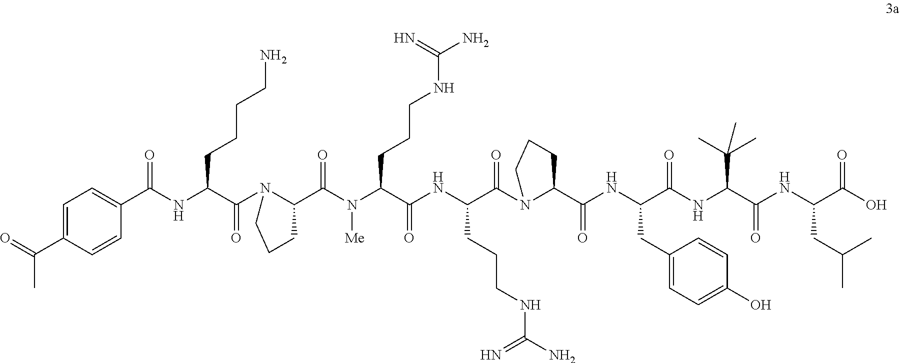 [SMILES: CC(=O)c1ccc(C(=O)N[C@@H](CCCCN)C(=O)N2CCC[C@H]2C(=O)N(C)[C@@H](CCCNC(=N)N)C(=O)N[C@@H](CCCNC(=N)N)C(=O)N2CCC[C@H]2C(=O)N[C@@H](Cc2ccc(O)cc2)C(=O)N[C@H](C(=O)N[C@@H](CC(C)C)C(=O)O)C(C)(C)C)cc1]

3a was synthesized by standard Fmoc chemistry by loading Fmoc-leucine onto 2-chlorotrityl resin (4 g, 0.5 mmol/g loading, 2.0 mmol), and subsequent coupling of Fmoc-tert-leucine, Fmoc-tyrosine (OtBu), Fmoc-proline, Fmoc-arginine (Pbf), Fmoc-N-methyl arginine (Pbf), Fmoc-proline, Fmoc-lysine (Boc), and finally 4-acetylbenzoic acid. The crude peptide was cleaved from the resin with 92.5:2.5:2.5:2.5 TFA:water:triisopropylsilane:DDT, triturated with MTBE, and the crude peptide purified by preparative HPLC (acetonitrile in water with 0.05% TFA) to provide 3a as the bis-TFA salt (148 mg, 0.0959 mmol, 4.8% yield).

The following compounds were made by a similar procedure:

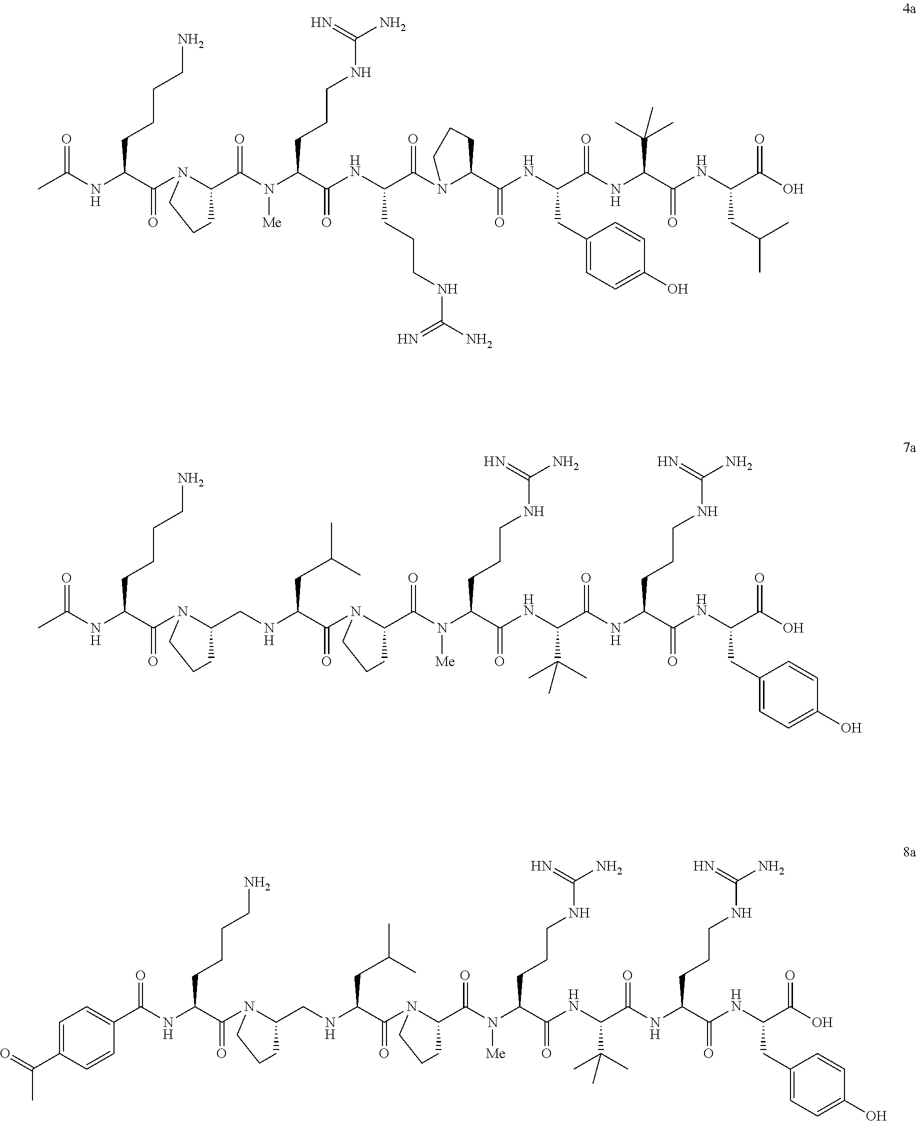

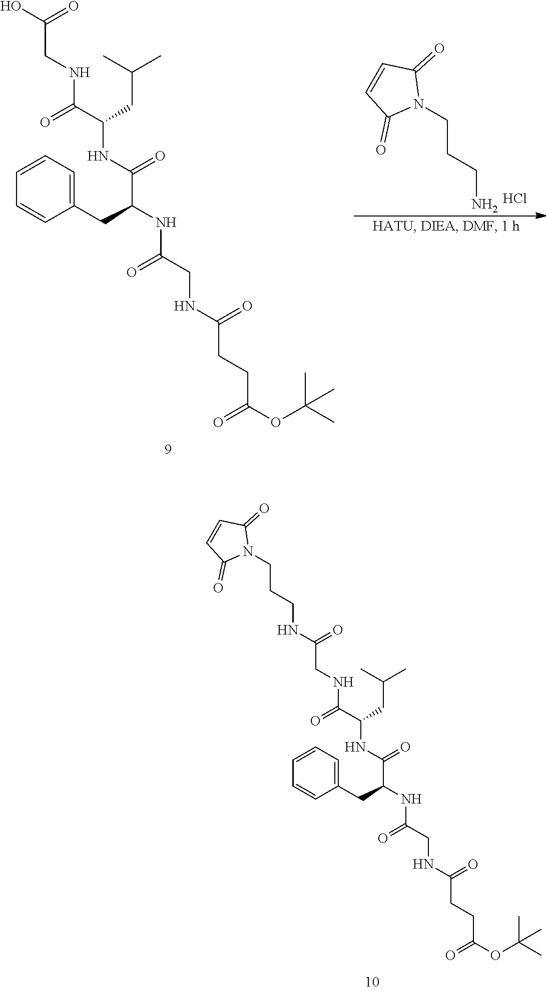 [SMILES: CC(C)C[C@H](NC(=O)[C@H](Cc1ccccc1)NC(=O)CNC(=O)CCC(=O)OC(C)(C)C)C(=O)NCC(=O)O ; NCCCN1C(=O)C=CC1=O ; Cl ; CC(C)C[C@H](NC(=O)[C@H](Cc1ccccc1)NC(=O)CNC(=O)CCC(=O)OC(C)(C)C)C(=O)NCC(=O)NCCCN1C(=O)C=CC1=O]

9 was synthesized on 2-chlorotrityl resin through standard Fmoc chemistry by appending Fmoc glycine, Fmoc leucine, Fmoc phenylalanine, Fmoc glycine, and mono-tert butyl succinate in that order, followed by cleavage from resin with 4:1 dichloromethane:hexafluoroisopropanol, and purification by HPLC.

9 (400 mg, 0.729 mmol), 3-aminopropylmaleimide hydrochloride (146 mg, 0.765 mmol), and HATU (416 mg, 1.09 mmol) were dissolved in DMF (4 mL) and diisopropylethylamine (0.382 mL, 2.19 mmol). The reaction was stirred at room temperature for 1 h, then the reaction mixture purified by preparative HPLC (water with 0.05% TFA/acetonitrile) to give 10 (230 mg, 0.336 mmol, 46% yield).

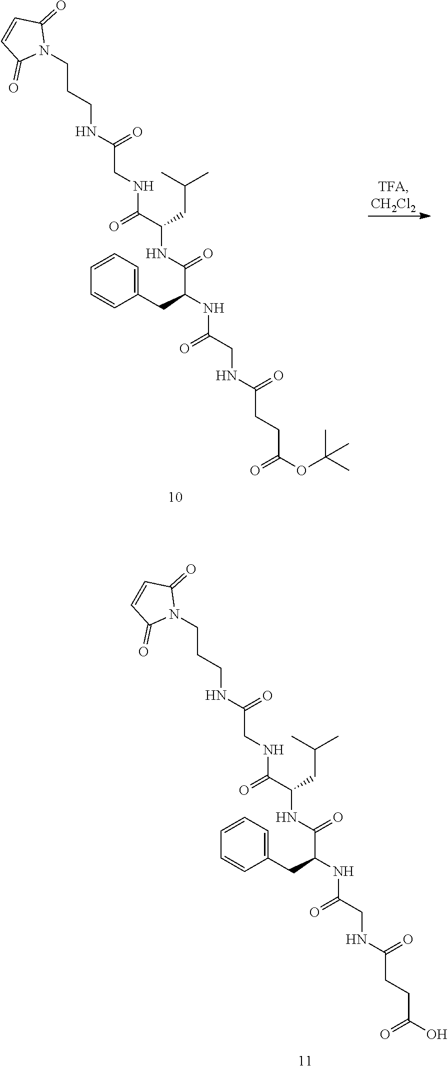 [SMILES: CC(C)C[C@H](NC(=O)[C@H](Cc1ccccc1)NC(=O)CNC(=O)CCC(=O)OC(C)(C)C)C(=O)NCC(=O)NCCCN1C(=O)C=CC1=O ; CC(C)C[C@H](NC(=O)[C@H](Cc1ccccc1)NC(=O)CNC(=O)CCC(=O)O)C(=O)NCC(=O)NCCCN1C(=O)C=CC1=O]

10 (230 mg, 0.336 mmol) was dissolved in 1:1 TFA:dichloromethane (6 mL), and the solution stirred at room temperature for 1 h. The solvent was removed in vacuo, and the remaining residue taken up in DMF (1 mL), and purified by preparative HPLC (water with 0.05% TFA/acetonitrile) to give 11 (108 mg, 0.172 mmol, 51% yield).

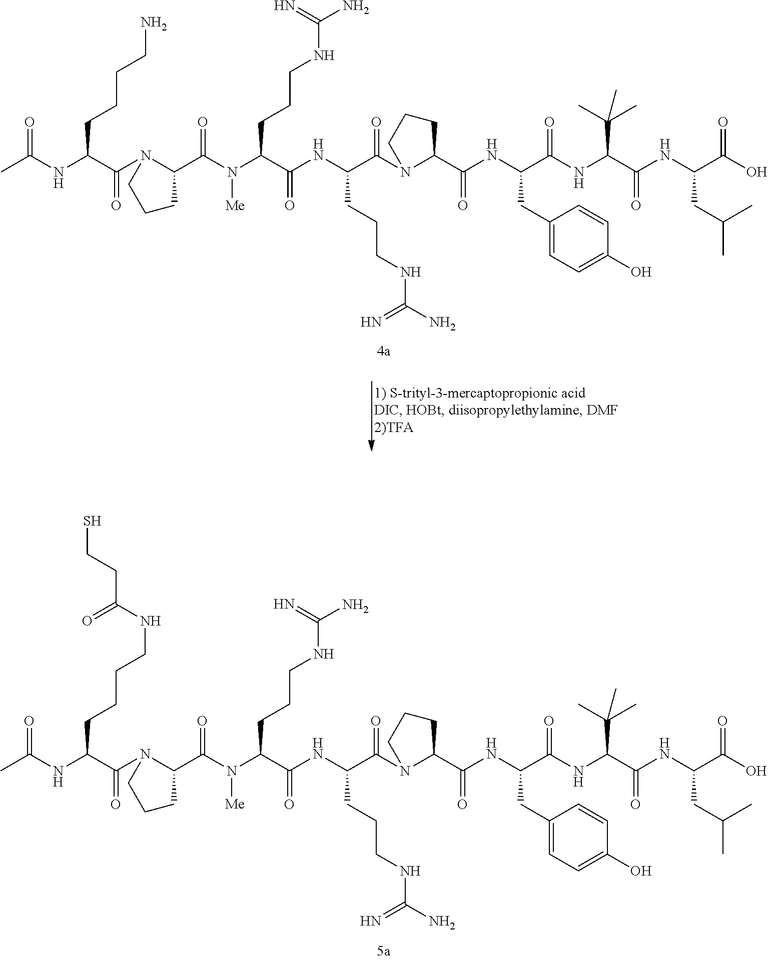

To a solution of 4a trifluoroacetate salt (200 mg, 0.139 mmol) was added S-trityl-3-mercaptopropionic acid (76.1 mg, 0.218 mmol), diisopropylcarbodiimide (34.5 mg, 0.273 mmol), HOBt (36.9 mg, 0.273 mmol), DMF (5 mL), and diisopropylethylamine (47.1 mg, 0.364 mmol). The reaction was stirred at room temperature for 3 h, the solvent removed in vacuo, and the remaining residue dissolved in trifluoroacetic acid (4 mL). The reaction was stirred at room temperature for 2 h, then diethyl ether (50 mL) was poured into the mixture. The resulting suspension was filtered, the solid washed with diethyl ether (2×30 mL), and the crude material purified by preparative HPLC (water/acetonitrile with 0.05% trifluoroacetic acid) to give 5a trifluoroacetate salt (75.8 mg, 0.0536 mmol, 38% yield). LCMS M/Z=1186.7 (M+1).

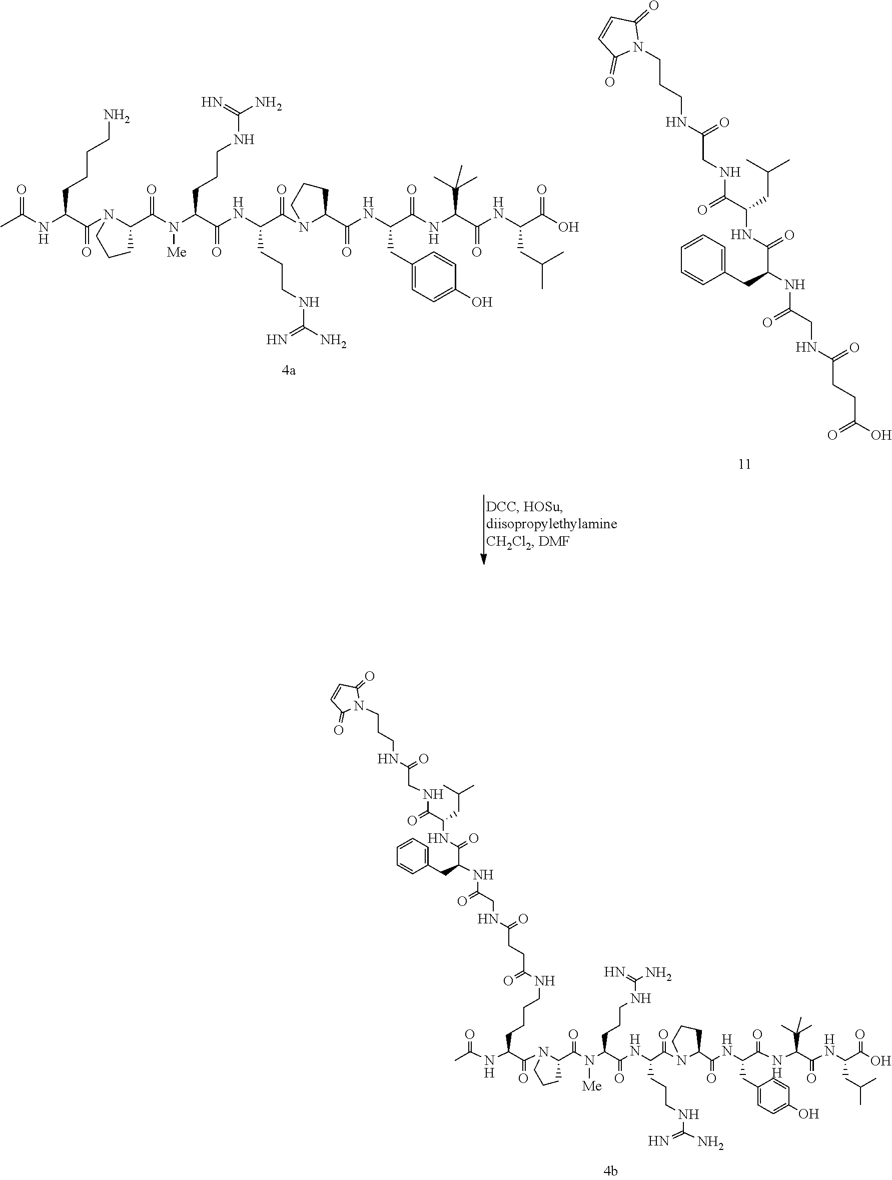

11 (45.8 mg, 72.8 μmol) was dissolved in dichloromethane (5 mL), and dicyclohexylcarbodiimide (18.0 mg, 87.4 μmol) and N-hydroxylsuccinimide (10.1 mg, 87.4 μmol) were added. The mixture was stirred at room temperature for 3 h. The solvent was removed, and a solution of 4a trifluoroacetate salt (80.0 mg, 55.6 μmol) in DMF (5 mL) was added. Diisopropylethylamine (25.5 μL, 145 μmol) was added, and the reaction stirred at room temperature for 3 h, then purified by reverse phase chromatography (water/acetonitrile with 0.05% trifluoroacetic acid) to give 4b trifluoroacetate salt (44.1 mg, 22.8 μmol, 41% yield). LCMS M/Z=854.6 [(M+2)/2], 570.2 [(M+3)/3].

The following compounds were made through a similar procedure:

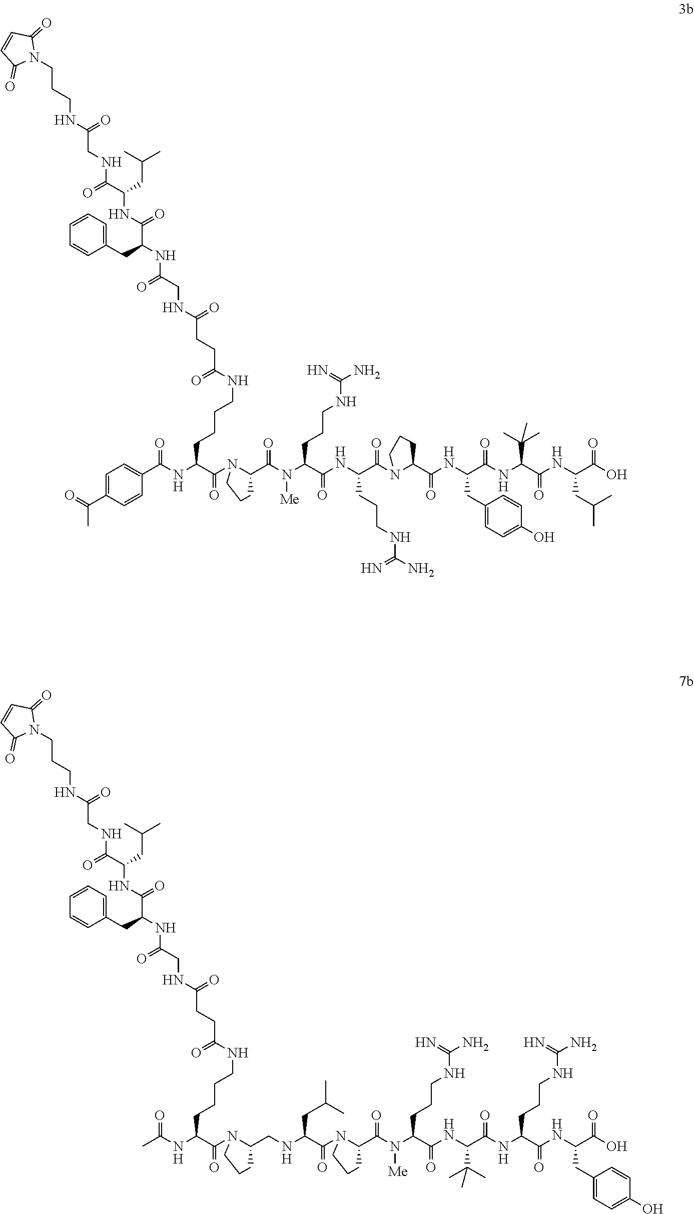

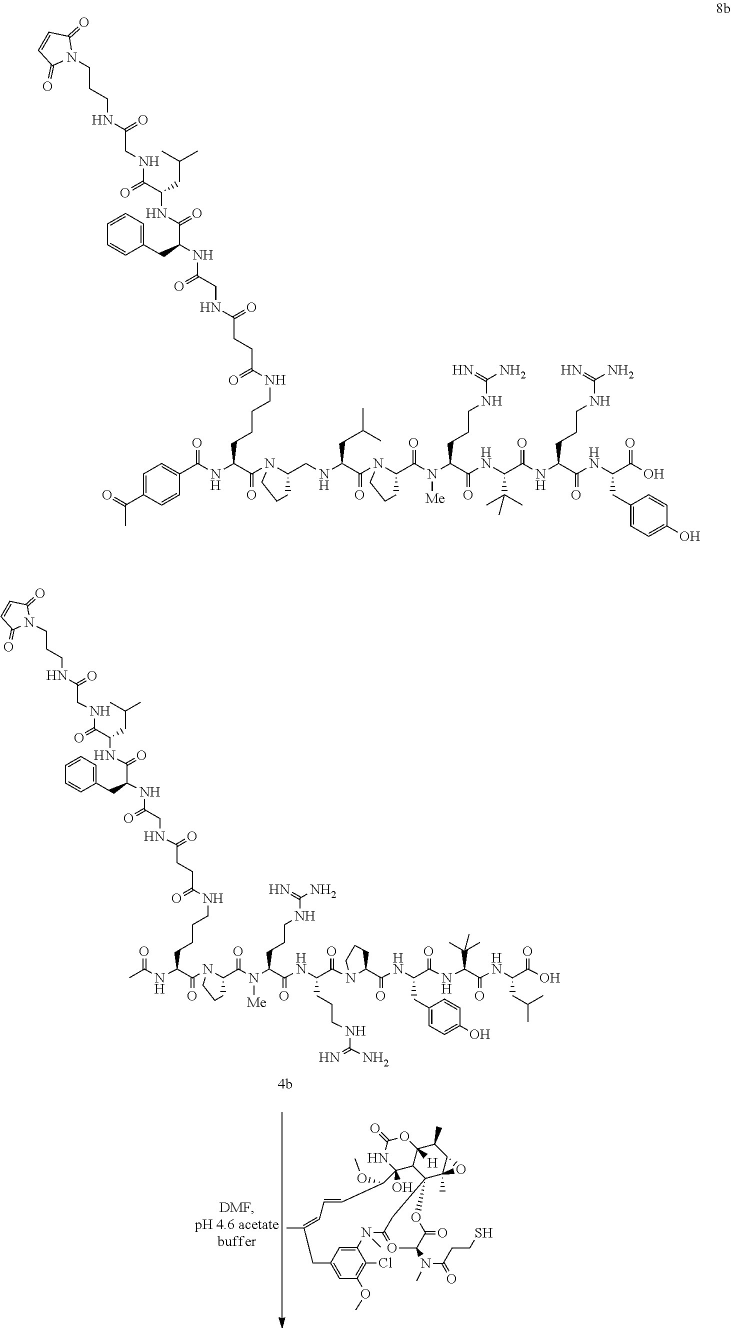

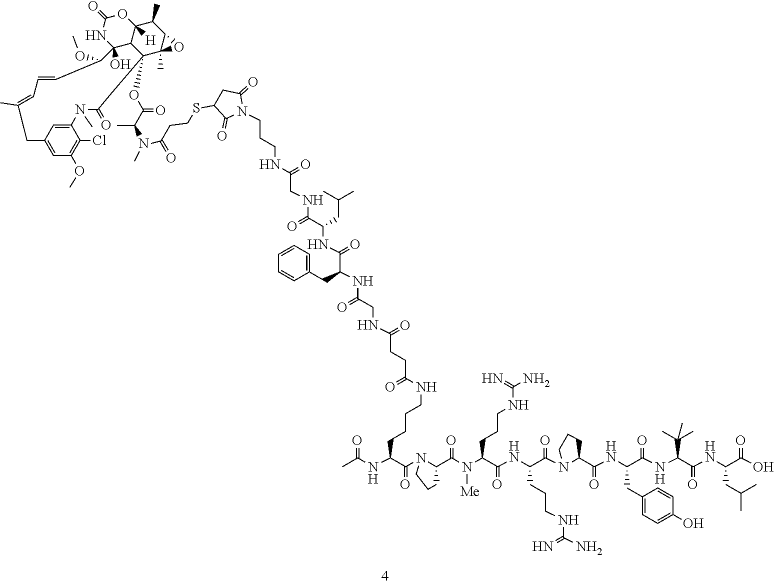

A vial was charged with 4b trifluoroacetate salt (44.1 mg, 22.8 μmol), and a solution of DM1 (28.1 mg, 38.0 μmol) in DIVIF (2 mL) was added. pH 4.6 acetate buffer (2.0 mL, made from equal volumes of 0.2M acetic acid and 0.2M sodium acetate) was added, the reaction stirred at room temperature for 1 h, and the reaction mixture purified by preparative HPLC (water/acetonitrile with 0.1% trifluoroacetic acid) to give 4 trifluoroacetate salt (54.1 mg, 20.2 μmol, 89% yield). LCMS M/Z=816.2 [(M+3)/3], 812.2 [(M+3−H₂O)/3], 795.5 [(M+3−H₂O−CO₂)/3].

The following compounds were made by a similar procedure: 3′, 7, 8′.

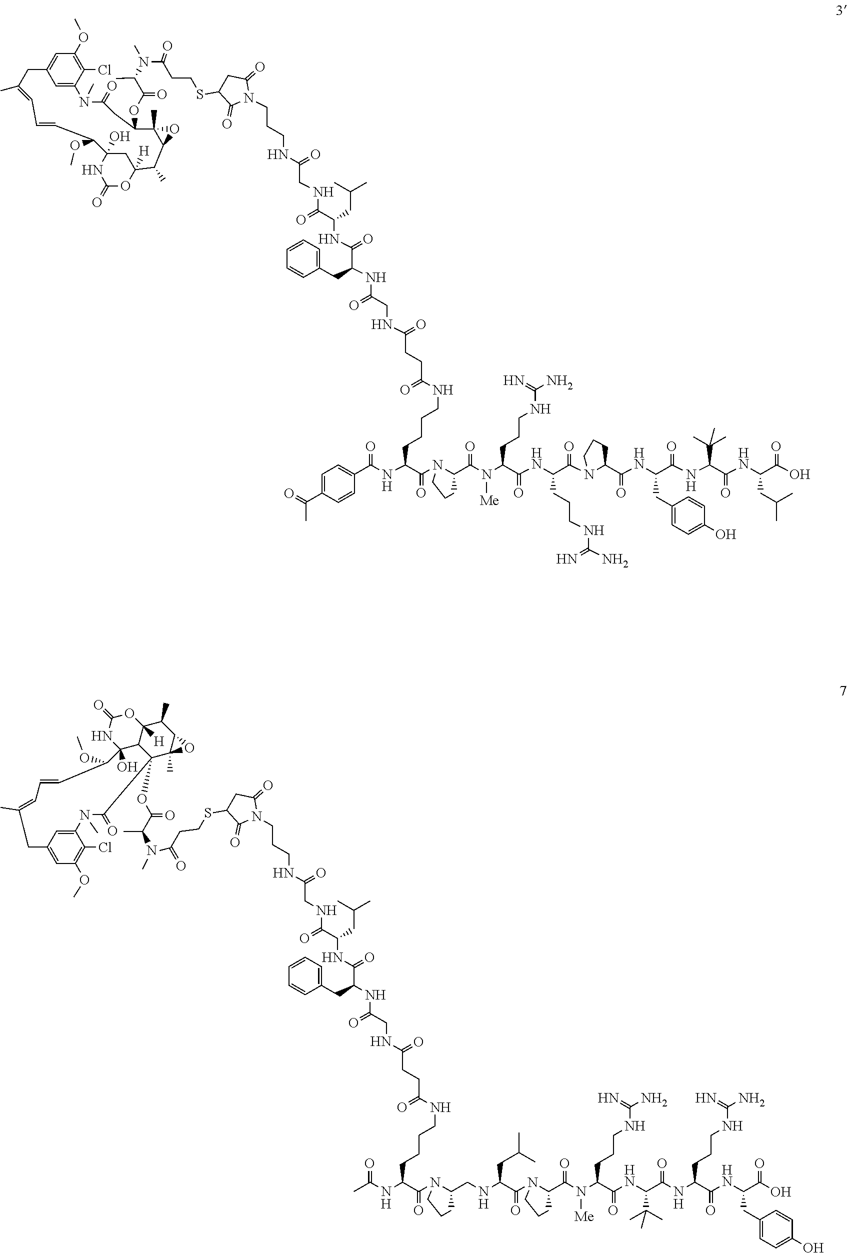

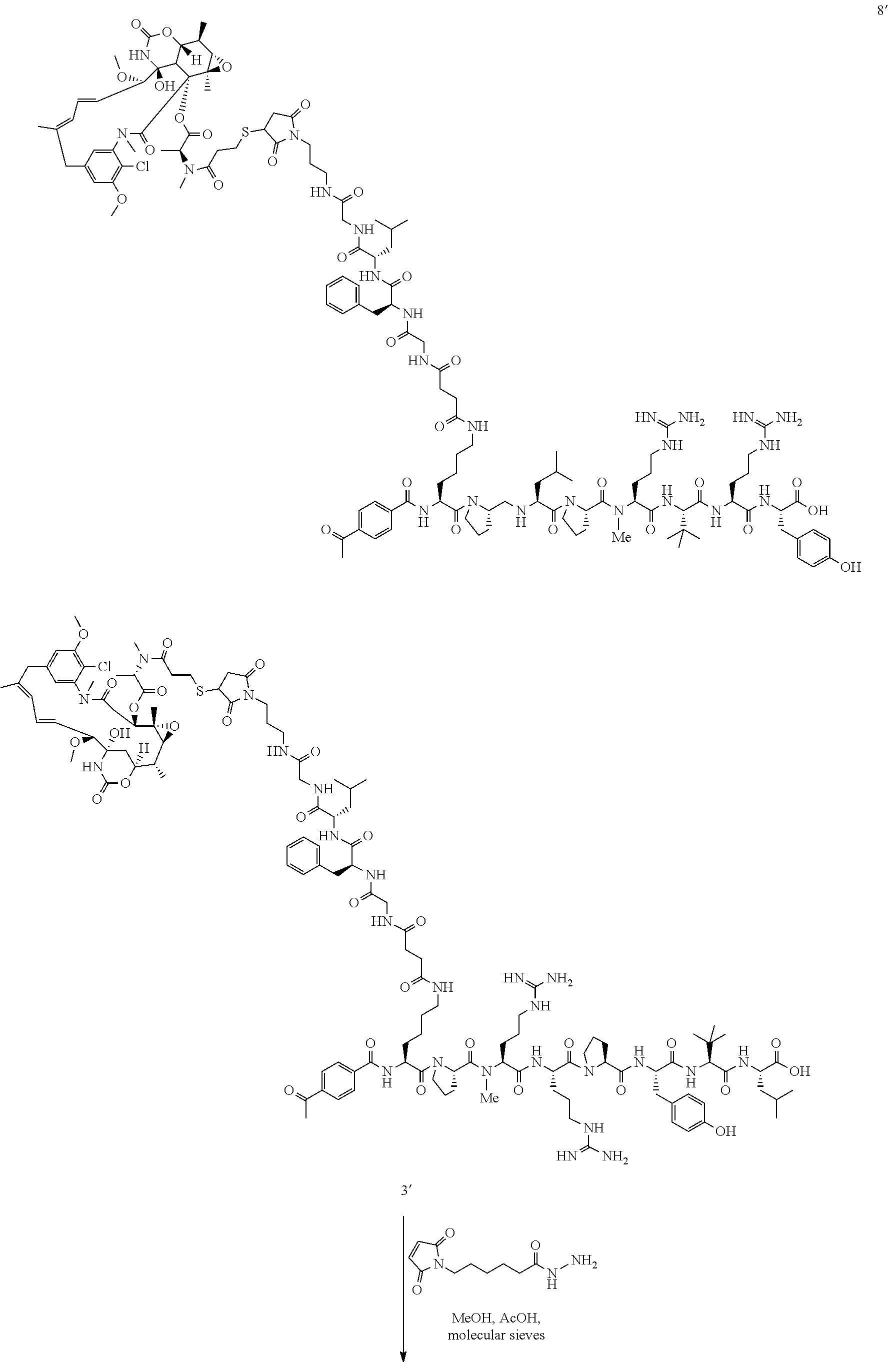

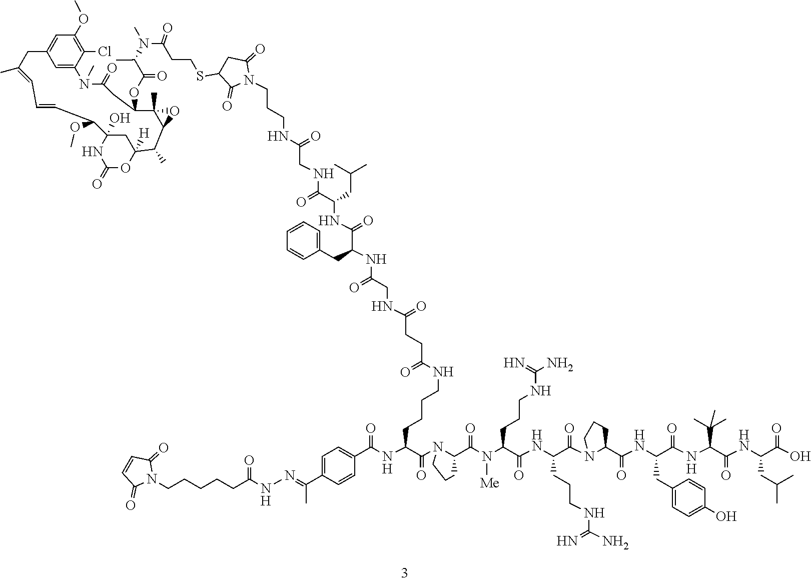 [SMILES: COc1cc2cc(c1Cl)N(C)C(=O)C[C@H](OC(=O)[C@H](C)N(C)C(=O)CCSC1CC(=O)N(CCCNC(=O)CNC(=O)[C@H](CC(C)C)NC(=O)[C@H](Cc3ccccc3)NC(=O)CNC(=O)CCC(=O)NCCCC[C@H](NC(=O)c3ccc(/C(C)=N/NC(=O)CCCCCN4C(=O)C=CC4=O)cc3)C(=O)N3CCC[C@H]3C(=O)N(C)[C@@H](CCCNC(=N)N)C(=O)N[C@@H](CCCNC(=N)N)C(=O)N3CCC[C@H]3C(=O)N[C@@H](Cc3ccc(O)cc3)C(=O)N[C@H](C(=O)N[C@@H](CC(C)C)C(=O)O)C(C)(C)C)C1=O)[C@]1(C)O[C@H]1[C@H](C)[C@@H]1C[C@@](O)(NC(=O)O1)[C@H](OC)/C=C/C=C(\C)C2]

A vial was charged with 3′ trifluoroacetate salt (36.2 mg, 13.0 μmol) and 6-maleimidohexanoic acid hydrazide (35.3 mg, 104 μmol). Anhydrous methanol (4 mL) and acetic acid (0.20 mL) were added, followed by dried 3A molecular sieves (100 mg). LCMS analysis was done on a C18 column with solvent A=50 mM ammonium bicarbonate buffered with formic acid to pH 7.0 (˜275 uL formic acid/L of buffer), and acetonitrile as solvent B. Gradient: 0 min, 5% B, 0.5% min 5% B, 1 min 35% B, 5.5 min 75% B, 6 min, 95% B. While starting material 3′ coeluted with product 3 on this method, reaction completeness could be judged by disappearance of starting material mass, M/Z=1276 [(M+2)/2]. Upon completion of the reaction after 4 h at room temperature, the reaction mixture was filtered through a short plug of anhydrous sodium sulfate, washing the plug with 2×2 mL anhydrous methanol. The filtrate was concentrated in vacuo, and the remaining oil taken up in DIVIF (3 mL) and purified by C18 chromatography, using the same aqueous ammonium bicarbonate/acetonitrile system as above. Lyophilization of the product-containing fractions yielded 3 free base (23.4 mg, 8.48 μmol), 65% yield). LCMS M/Z=1379 [(M+2)/2].

Compound 8 was made by a similar procedure.

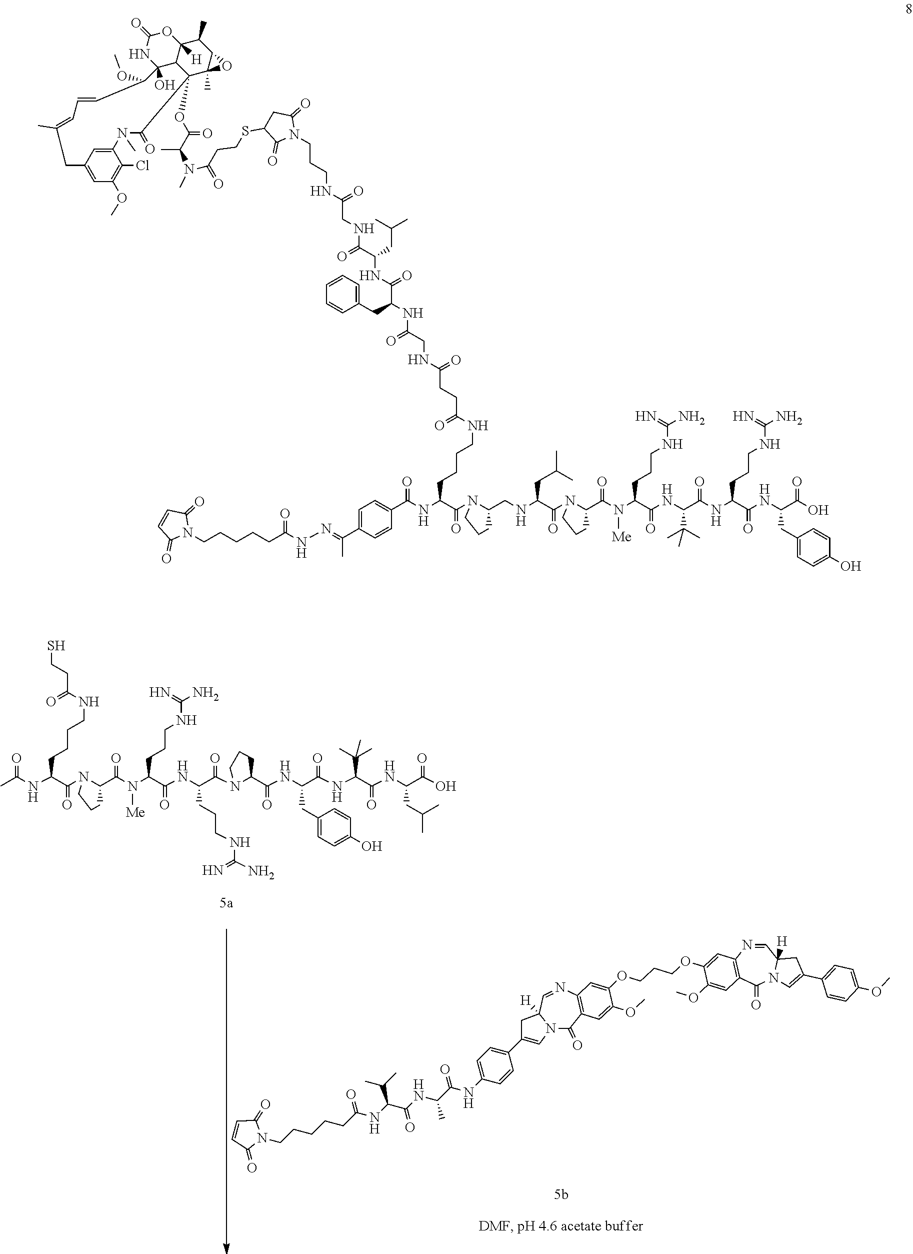

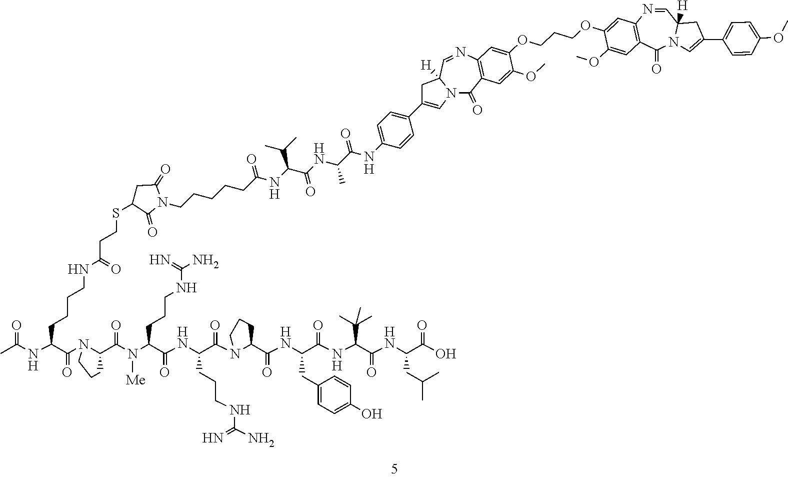 [SMILES: COc1ccc(C2=CN3C(=O)c4cc(OC)c(OCCCOc5cc6c(cc5OC)C(=O)N5C=C(c7ccc(NC(=O)[C@H](C)NC(=O)[C@@H](NC(=O)CCCCCN8C(=O)CC(SCCC(=O)NCCCC[C@H](NC(C)=O)C(=O)N9CCC[C@H]9C(=O)N(C)[C@@H](CCCNC(=N)N)C(=O)N[C@@H](CCCNC(=N)N)C(=O)N9CCC[C@H]9C(=O)N[C@@H](Cc9ccc(O)cc9)C(=O)N[C@H](C(=O)N[C@@H](CC(C)C)C(=O)O)C(C)(C)C)C8=O)C(C)C)cc7)C[C@H]5C=N6)cc4N=C[C@@H]3C2)cc1]

5a was charged into a vial, and an solution of 5b (1 equiv.) in DMF was added. pH 4.6 acetate buffer was subsequently added, the reaction stirred at room temperature for 1 h, and the crude reaction mixture purified by preparative HPLC (water/acetonitrile with 0.05% trifluoroacetic acid) to give 5 as the trifluoroacetate salt. LCMS M/Z=759.2 [(M+3)/3].

Compound 6 was made by a similar procedure.

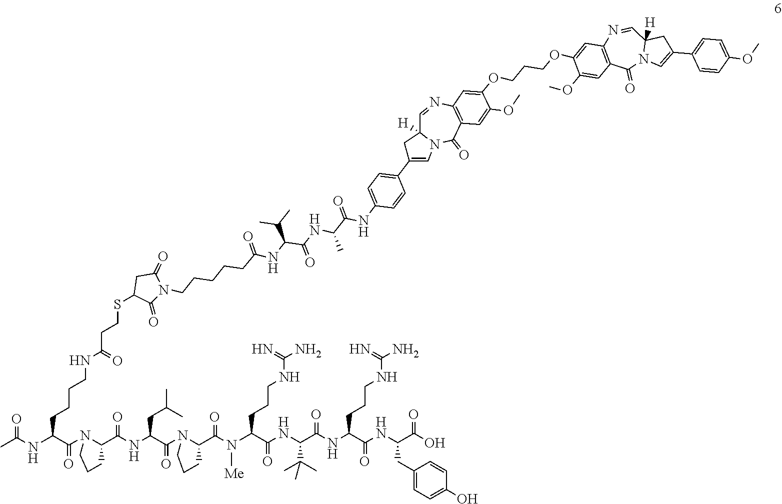 [SMILES: COc1ccc(C2=CN3C(=O)c4cc(OC)c(OCCCOc5cc6c(cc5OC)C(=O)N5C=C(c7ccc(NC(=O)[C@H](C)NC(=O)[C@@H](NC(=O)CCCCCN8C(=O)CC(SCCC(=O)NCCCC[C@H](NC(C)=O)C(=O)N9CCC[C@H]9C(=O)N[C@@H](CC(C)C)C(=O)N9CCC[C@H]9C(=O)N(C)[C@@H](CCCNC(=N)N)C(=O)N[C@H](C(=O)N[C@@H](CCCNC(=N)N)C(=O)N[C@@H](Cc9ccc(O)cc9)C(=O)O)C(C)(C)C)C8=O)C(C)C)cc7)C[C@H]5C=N6)cc4N=C[C@@H]3C2)cc1]

The following two albumin binding conjugates were synthesized for comparison purposes. They do not bind to NTSR1.

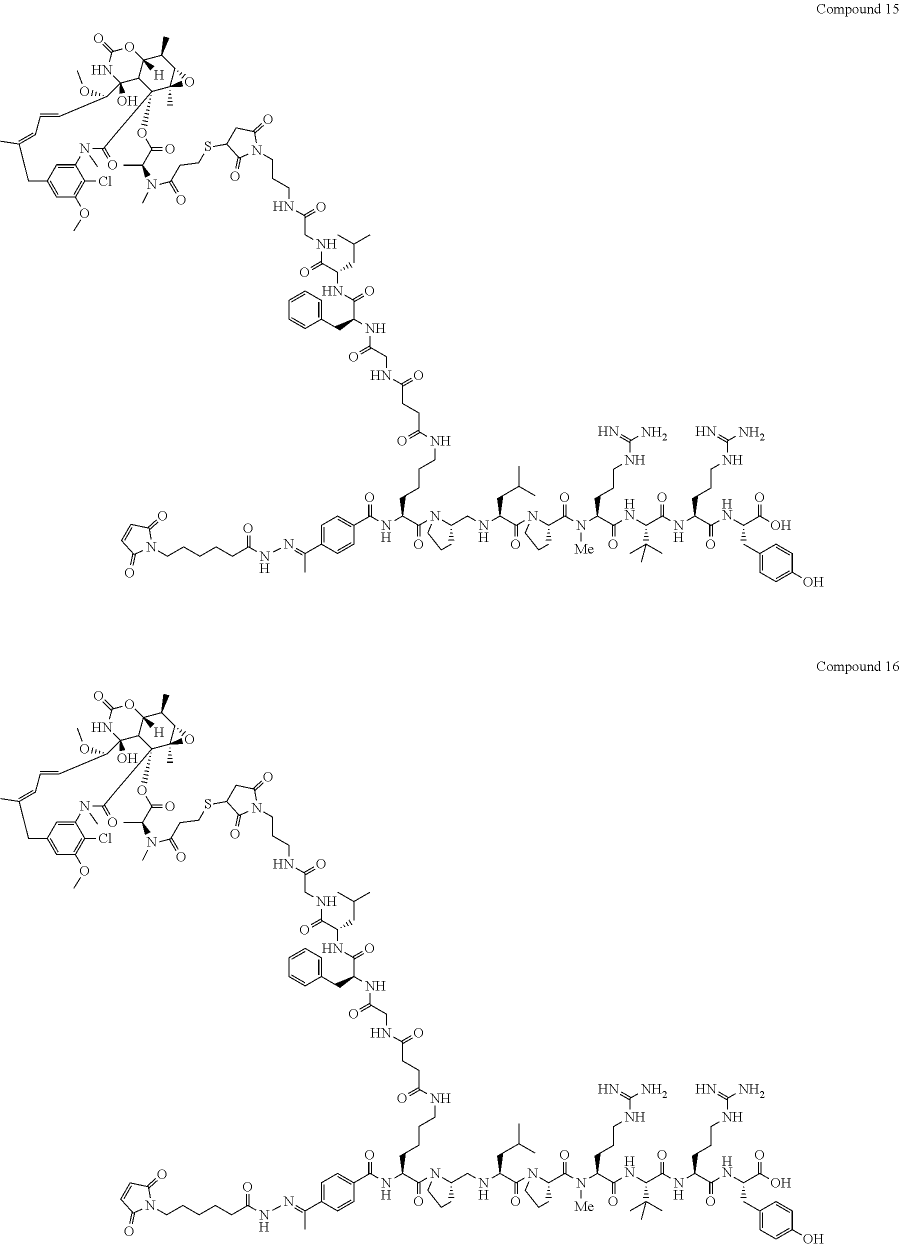

### Example 5

### In Vivo Half-Life of Albumin Binding Conjugates

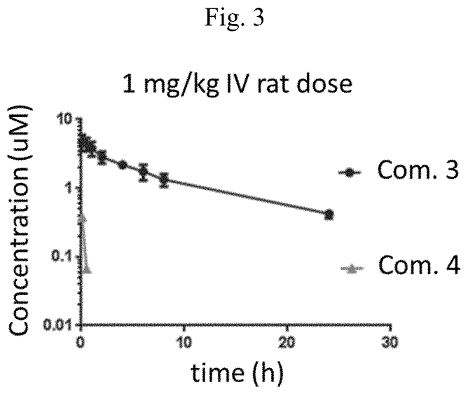

In vivo half-lives of an albumin binding NTS-DM1 conjugate, Compound 3, and its equivalent NTS-DM1 conjugate, Compound 4, which does not bind to albumin, were compared. Compound 3 and Compound 4 were administered to rats at 1 mg/kg via IV. Concentration changes of Compounds 3 and 4 in plasma were shown in FIG. 3. Compound 3 binds to albumin and is converted to Compound 3′ when the pH is less than 7. Therefore, the concentration of Compound 3′ is measured after treating plasma samples at a low pH. Compound 4 degraded faster than the albumin bound Compound 3 measured as Compound 3′ after release. The rat plasma half-life of Compound 3 is 80-fold longer (9.3 hours) than Compound 4 that does not bind albumin.

Therefore, covalent albumin binding significantly increases in vivo half life of the conjugate. Pharmacokinetics data of Compound 3 was shown in Table 1.

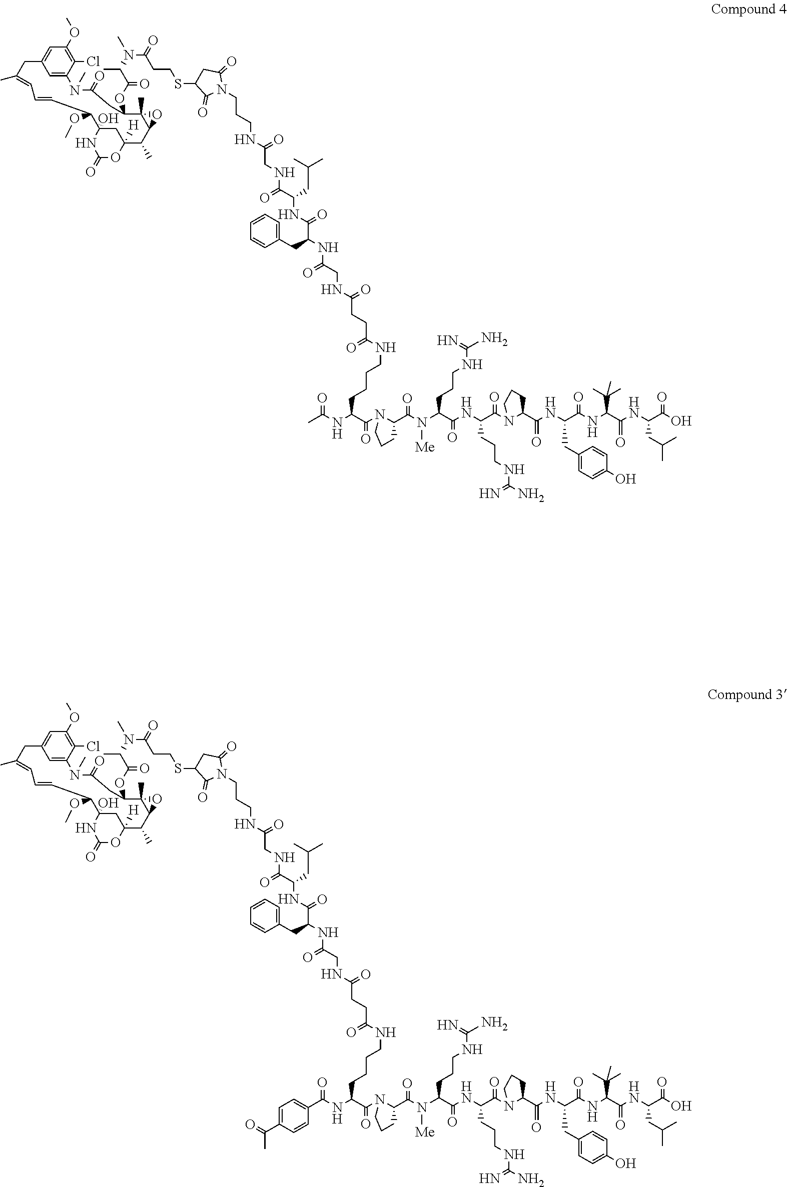

TABLE 1
Pharmacokinetics of Compound 3

| NCA pharmacokinetics | | |
|---|---|---|
| Parameter | Units | Compound 3 |
| Dose | mg/kg | 1.0 |
| t½ | h | 9.32 |
| Cmax | uM | 4.69 |
| CL | mL/kg/min | 0.16 |
| AUC | uM * h | 33.7 |
| AUC inf | uM * h | 39.3 |
| Vss | mL/kg | 107 |

### Example 6

### Efficacy Study of Albumin Binding Conjugates

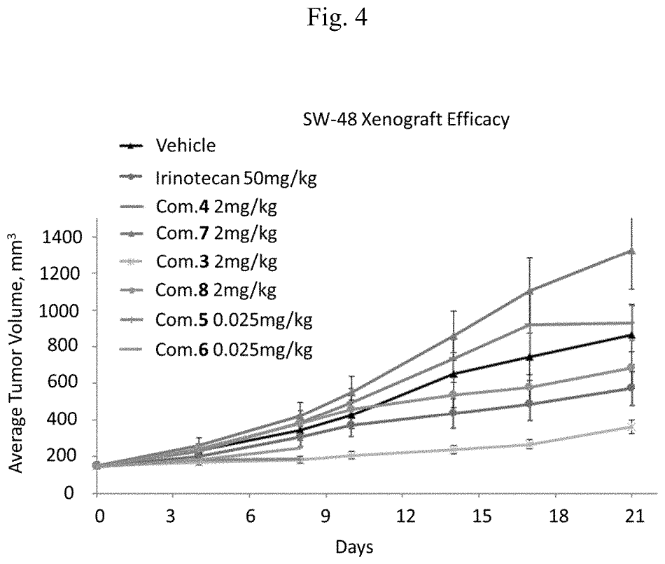

Efficacy of Compound 3 was tested in an SW48 xenograft model (human colorectal cancer). Efficacies of the other compounds in Table 2 were also tested. All conjugates were dosed twice/week for two weeks. Irinotecan was dosed once/week for two weeks. Average tumor volume changes were shown in FIG. 4. Tumor growth inhibition (TGI %) data were shown in Table 3. Compound 3 generated greater TGI % than any other compound.

TABLE 2
Conjugates tested in the efficacy study

| | | | | | | | | IC50, SW48 | |
|---|---|---|---|---|---|---|---|---|---|
| Compound | Targeting ligand | Payload | Albumin-bound? | Dose | | Once/week MTD | | cells | |
| Vehicle | n/a | n/a | n/a | n/a | | n/a | | n/a | |
| Irinotecan | n/a | SN-38 | n/a | 50 | mg/kg | 50 | mg/kg | n.d. | |
| 5 | NTSR1 ligand | PBD dimer | no | 0.025 | mg/kg | 0.05 | mg/kg | 1.8 | nM |
| 6 | scrambled NTSR1 | PBD dimer | no | 0.025 | mg/kg | 0.05 | mg/kg | 4.0 | nM |
| | ligand | | | | | | | | |
| 4 | NTSR1 ligand | DM1 | no | 2.0 | mg/kg | >8 | mg/kg | 3.7 | nM |
| 7 | Scrambled NTSR1 | DM1 | no | 2.0 | mg/kg | >8 | mg/kg | 35 | nM |
| | ligand | | | | | | | | |

| | | | | | | | | |
|---|---|---|---|---|---|---|---|---|
| 3 | NTSR1 | DM1 | yes | 2.0 | mg/kg | between 4-8 | 9.3 | nM* |
| | | | | | | mg/kg | | |
| 8 | scrambled NTSR1 | DM1 | yes | 2.0 | mg/kg | between 4-8 | 55 | nM* |
| | ligand | | | | | mg/kg | | |

*IC50s are for Compound 3′ (T-1544) and Compound 8′ (T-1535), respectively, which are products of acid-mediated cleavage of each conjugate from albumin.

TABLE 3
Tumor growth inhibition by conjugates

| | TGI % | | | | | |
|---|---|---|---|---|---|---|
| Treatment | Day 4 | Day 8 | Day 10 | Day 14 | Day 17 | Day 21 |
| Irinotecan | 14.1 | 10 | 12.8 | 33 | 34.9 | 33.9 |
| 4 | 1.8 | −12.5 | −16.5 | −13.2 | −23.7 | −7.3 |
| 7 | −11 | −22.6 | −29.7 | −32 | −48.3 | −52.8 |
| 3 | 26.9 | 46.8 | 51 | 63.5 | 64 | 58.2 |
| 8 | −3.2 | −11.5 | −8.5 | 17.6 | 22.5 | 21.1 |
| 5 | 21.3 | 45.9 | n/a | n/a | n/a | n/a |
| 6 | 22.2 | 28.3 | n/a | n/a | n/a | n/a |

### Example 7

### Blood Pressure Study of an Albumin Binding Conjugate

Blood pressure changes induced by neurotensin and by conjugates was investigated in Sprague Dawley rats equipped for intravenous infusion via a catheter implanted in a femoral vein. An external pumping device was used to deliver the target dose volume for each animal over a 10 minute dosing interval. Blood pressure was measured by tail cuff using the CODA Non-Invasive Blood Pressure system. Animals were placed in restrainers with the tail cuff on. The animals and the restrainers were then placed on a heating pad for approximately 5 minutes. Following the 5 minute heating pad acclimation an additional acclimation data collection took place prior to dose initiation for approximately 5 minutes. Blood pressure data began at dose initiation and was collected through at least 30 minutes postdose. Groups were dosed according to Table 4. Test articles were infused over 10 minutes. The start and stop times for the following processes were documented. The following parameters were collected: diastolic pressure, systolic pressure, mean blood pressure, heart rate, tail blood flow, and tail blood volume.

TABLE 4
Conjugates tested in Sprague Dawley rat blood pressure study

| | No. of | | Dose | |
|---|---|---|---|---|
| | Animals | Dose Level | Concentration | Dose Rate |
| Group | Female | (nmol/kg) | (mg/mL) | (mL/kg/hr) |
| 1 Vehicle | 6 | 0 | 0 | |
| 2 Neurotensin | 6 | 2.50 | 0.005 | 60 |
| 3 Compound 3 | 6 | 400 | 0.1 | 60 |
| 4 Compound 4 | 6 | 400 | 0.1 | 60 |

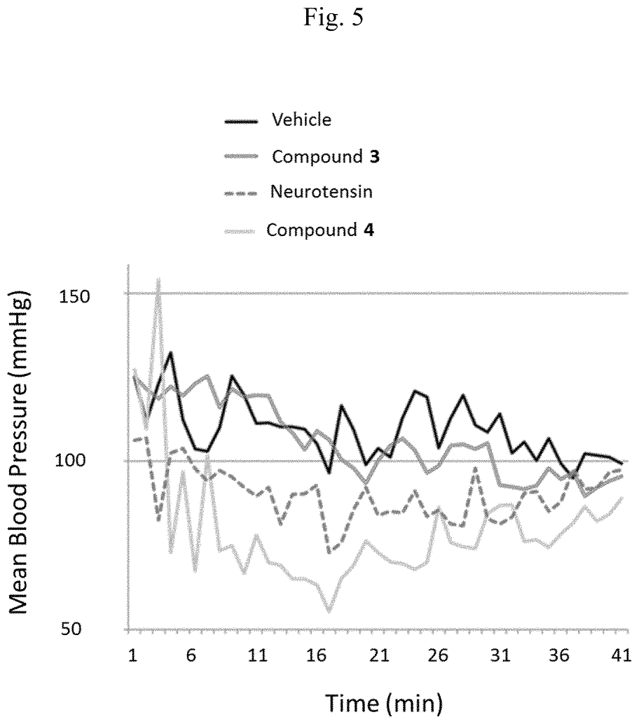

The mean blood pressure data for all four groups in Table 4 are shown in FIG. 5. Neurotensin and Compound 4 (which does not form a covalent link to albumin) both caused a significant drop in mean blood pressure over the 10 minute infusion which gradually recovered over the remaining approximately 30 minutes of measurement. A significant drop, as used here, refers to a drop of at least 10%. In contrast, Compound 3 (which forms a covalent link to albumin) showed no significant drop in mean blood pressure, with a blood pressure profile indistinguishable from the vehicle (saline) infused rats. This study demonstrates that a conjugate that forms a covalent link to albumin shows less systemic toxicity, in this case a drop in mean blood pressure, than a related conjugate that does not form a covalent link to albumin.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control. Section and table headings are not intended to be limiting.

1. A targeted construct comprising at least one conjugate comprising an active agent coupled, via an optional internal linker, to a targeting moiety, wherein the targeted construct further comprises at least one reacting group for reacting with a functional group on a protein or an engineered protein or derivatives/analogs/mimics thereof.

2. The targeted construct of claim 1, wherein the reacting group is attached to the active agent moiety, the targeting moiety, or the optional internal linker moiety of the conjugate by an external linker.

3. The targeted construct of claim 2, wherein the external linker is cleavable.

4. The targeted construct of claim 3, wherein the external linker is cleavable in an extracellular manner.

5. The targeted construct of claim 4, wherein the external linker is acid-labile.

6. The targeted construct of claim 1, wherein the internal linker moiety comprises the reacting group.

7. The targeted construct of claim 1, wherein the protein is albumin.

8. The targeted construct of claim 6, wherein the reacting group is selected from the group consisting of a disulfide group, a vinylcarbonyl group, a vinyl acetylene group, an aziridine group, an acetylene group, and any of the following reacting groups: 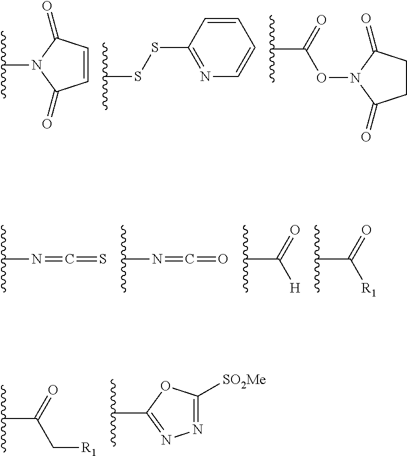
wherein R₁ is selected from the group consisting of Cl, Br, F, mesylate, tosylate, O-(4-nitrophenyl), and O-pentafluorophenyl.

9. The targeted construct of claim 1, wherein the protein is transthyretin.

10. The targeted construct of claim 1, wherein the reaction between the reacting group and the functional group happens in vivo.

11. The targeted construct of claim 1, wherein the reaction between the reacting group and the functional group is performed in vitro prior to administration.

12. The targeted construct of claim 1, wherein the conjugate comprises a formula selected from the group X-Y-Z, X-Y-Z-Y-X, X-(Y-Z)ₙ, Xₙ-Y-Z, (X-Y)ₙ-Z, Xₙ-Y-Z, X-Y-Zₙ, and (X-Y-Z-Y)ₙ-Z;
wherein X is the targeting moiety,
Y is the internal linker moiety,
Z is the active agent, and
n is an integer between 2 and 1,000.

13. The targeted construct of claim 12, wherein the conjugate comprises the formula X-Y-Z;
wherein X is the targeting moiety,
Y is the internal linker moiety, and
Z is the active agent.

14.-16. (canceled)

17. The targeted construct of claim 1, wherein the targeting moiety binds to neurotensin receptor (NTSR).

18. The targeted construct of claim 17, wherein the targeting moiety is neurotensin or a derivative or analog thereof.

19.-21. (canceled)

22. The targeted construct of claim 1, wherein the internal linker moiety is not a cleavable linker.

23. The targeted construct of claim 1, wherein the internal linker moiety is a cleavable linker.

24. The targeted construct of claim 23, wherein the internal linker moiety is cleaved in an intracellular manner.

25. The targeted construct of claim 23, wherein the internal linker is cleaved by a protease.

26. The targeted construct of claim 1, wherein the internal linker moiety comprises an ester bond, disulfide, amide, acylhydrazone, ether, carbamate, carbonate, or urea.

27.-29. (canceled)

30. The targeted construct of claim 1, wherein the active agent is a small molecule, protein, peptide, lipid, carbohydrate, sugar, nucleic acid, or combination thereof.

31. The targeted construct of claim 30, wherein the active agent is a small molecule.

32. The targeted construct of claim 31, wherein the active agent is cabazitaxel, DM1, PBD or a PBD dimer, or derivatives/analogs thereof.

33. The targeted construct of claim 31, wherein the active agent is tubulysin or its analog or derivative.

34. The targeted construct of claim 1, wherein the half-life of the targeted construct may be at least about 25%, 50%, 75%, 100%, 200%, or 500% more than the half-life of the conjugate itself.

35. The targeted construct of claim 1, wherein the conjugate comprises an active agent coupled to a targeting moiety by a cleavable internal linker moiety, wherein the targeted construct further comprises a maleimide group for reacting with a functional group on albumin.

36. The targeted construct of claim 35, wherein the active agent is DM1.

37. The targeted construct of claim 35, wherein the targeting moiety binds to NTSR1.

38. The targeted construct of claim 37, wherein the targeting moiety is neurotensin or a derivative or analog thereof.

39. The targeted construct of claim 34, wherein the conjugate is selected from the group consisting of Compound 3, Compound 12, or Compound 13.

40. A pharmaceutical formulation comprising the targeted construct of claim 1 and at least one pharmaceutically acceptable excipient.

41. A method of treating a subject in need thereof comprising administering a therapeutically effective amount of the formulation of claim 40.

42. The method of claim 41, wherein the subject has tumor.

43. The method of claim 42, wherein the tumor is lung cancer, breast cancer, colorectal cancer, neuroendodrine cancer, ovarian cancer, pancreatic cancer, colorectal cancer, bladder cancer, prostate cancer, cervical cancer, renal cancer, leukemia, central nervous system cancers, myeloma, or melanoma.

44. The method of claim 42, wherein the tumor volume is reduced.

45. The method of claim 42, wherein the blood pressure of the subjection does not drop significantly.
